(19) United States
(12) Patent Application Publication
(10) Pub. No.: US 2025/0043297 A1
(43) Pub. Date: Feb. 6, 2025

(54) USE OF POLYNUCLEOTIDE, PROTEIN, AND BIOLOGICAL MATERIAL IN REGULATION AND CONTROL OF PLANT TUBER DEVELOPMENT, AND RELATED PRODUCT AND PRODUCTION METHOD THEREFOR

(71) Applicants: Agricultural Genomics Institute at Shenzhen, Chinese Academy of Agricultural Sciences, Shenzhen (CN); Institute of Vegetables and Flowers, Chinese Academy of Agricultural Sciences, Beijing (CN)

(72) Inventors: Sanwen HUANG, Shenzhen (CN); Jinzhe ZHANG, Shenzhen (CN); Zhihong LIU, Shenzhen (CN); Yuxin JIA, Shenzhen (CN); Shuangshuang FENG, Shenzhen (CN)

(73) Assignees: Agricultural Genomics Institute at Shenzhen, Chinese Academy of Agricultural Sciences, Shenzhen (CN); Institute of Vegetables and Flowers, Chinese Academy of Agricultural Sciences, Beijing (CN)

(21) Appl. No.: 18/822,127

(22) Filed: Aug. 31, 2024

Related U.S. Application Data
(63) Continuation of application No. PCT/CN2023/083321, filed on Mar. 23, 2023.

(51) Int. Cl. C12N 15/82; A01H 6/82; C12N 9/22

(52) U.S. Cl. CPC C12N 15/8226; A01H 6/827; C12N 9/22; C12N 2310/20

(57) ABSTRACT

Disclosed are use of a polynucleotide, protein, and biological material in the regulation and control of plant tuber development, and related products and production methods thereof. The tuber plant or part thereof, comprising a sequence encoding a polypeptide comprising a sequence having at least 92% sequence identity to SEQ ID NO: 2, wherein the polypeptide is inhibited-expressed, loss-function, or overexpressed, and the development of plant tuber is changed. The polynucleotide is an identity gene of a tuber, and can control whether a tuber is formed, the formation time, the tuber size etc. by regulating and controlling a protein function, an expression quantity etc.

### CROSS-REFERENCE TO RELATED APPLICATIONS

The present disclosure is a continuation (CA) of International Application NO. PCT/CN2023/083321, filed on Mar. 23, 2023, which claims priority to Chinese Patent Application No. 202210205170.8, filed on Mar. 2, 2022. The disclosures of the aforementioned applications are hereby incorporated by reference in their entireties.

### REFERENCE TO AN ELECTRONIC SEQUENCE LISTING

The contents of the electronic sequence listing (SequenceListing.xml; Size: 330,976 bytes; and Date of Creation: Oct. 23, 2024) is herein incorporated by reference in its entirety.

### TECHNICAL FIELD

The present disclosure relates to the field of plant genetic engineering, and in particular to the use of polynucleotides, proteins, and biological materials in the regulation of the development of plant tubers and related products and production methods.

### BACKGROUND

Potato is derived from the Andes Mountains of South America and is an annual dicotyledonous solanum plant in the Solanaceae family. Potato tubers are metamorphic organs formed by the enlargement of the tops of underground stolon. They are both the reproductive and product organs of potatoes. Therefore, the study of the formation mechanism of potato tubers not only plays an important guiding role in the application of seed potato breeding, increasing potato yield, and improving quality traits, but is also a focus of research on the development of plant metamorphic organs and has important scientific significance. Under natural conditions, the formation of potato tubers is divided into two independent biological processes: the formation of stolons and the expansion of the tops of stolons. They must go through the 5 steps of: occurrence of stolons, elongation of stolons, cessation of longitudinal growth of stolons, radial growth of the tops of stolons, and occurrence and enlargement of tubers.

Although scientists have done a lot of research on the development of potato tubers, the formation of potato tubers is a very complex biological process and is the result of a combination of endogenous signals in plants and the external environment. The key regulatory genes for the formation of potato tubers need to be further discovered, particularly the identity genes that control tuber formation have not yet been reported.

### SUMMARY

In view of this, the present disclosure provides use of a polynucleotide, protein, and biological material in the regulation of development of a plant tuber, and the related products and production methods. The polynucleotide IT1 is the identity gene of the tuber which controls whether or not to form tubers, formation time of tubers, the number of the tubers and the size of tubers by regulating the protein function and the expression level.

In order to achieve the above object of the disclosure, the present disclosure provides the following technical solutions.

In one aspect, the disclosure provides the use of a polynucleotide in the regulation of development of plant tuber, which includes at least one of the following nucleotide sequences:
1) the nucleotide sequence represented by SEQ ID NO:1;
2) the complementary sequence, degenerate sequence, or homologous sequence of the sequence represented by SEQ ID NO:1, where the homologous sequence is a polynucleotide having 75% or more identity with the nucleotide sequence represented by SEQ ID NO:1;
3) a polynucleotide hybridizing to the nucleotide sequence represented by SEQ ID NO: 1 under stringent conditions or its complementary sequence;
4) the cDNA sequence of any one of the sequences 1)-3);
5) the nucleotide sequence represented by SEQ ID NO:3;
6) the complementary sequence, degenerate sequence, truncated sequence, or homologous sequence of the sequence represented by SEQ ID NO:3, where the homologous sequence is a polynucleotide having 75% or more identity with the nucleotide sequence represented by SEQ ID NO: 3;
7) a polynucleotide hybridizing to the nucleotide sequence represented by SEQ ID NO: 3 under stringent conditions or its complementary sequence;
8) the cDNA sequence of any of the sequences 5)-7).

By analyzing RNA-seq data of different potato tissues, the present disclosure identifies a TCP transcription factor IT1 that is predominantly expressed in potato tubers. By constructing the site-directed mutation vector CRISPR-IT1 based on the CRISPR/Cas9 system, the site-directed mutation vector was transferred into potato plants by using the agrobacterium-mediated transgene system, and transgenic positive lines were obtained through resistance screening and PCR verification. For the transgenic lines with site-directed mutations, DNA of the plants was extracted, and lines with loss-function of IT1 were screened out by PCR and sequencing methods. The screened transgenic lines were used for subsequent phenotypic observation. It was found that the formation of tuber in transgenic line with the IT1 function-deficient was seriously affected. The stolons did not further expand to form tubers, but grew upward to form a new plant. A potato material without forming tubers under normal growing conditions was created by knock-off of IT1.

IT1 is a TB-like TCP transcription factor. Its homologous genes have been reported to function in Arabidopsis, corn and other crops to regulate plant branching, and its function loss can increase plant branching. This is the first discovery in this disclosure that IT1 regulates development of potato tubers, and its loss-function can cause potato stolons to not develop into tubers but to grow upward to form new plants. This phenotype is reported for the first time in potatoes, and it is the identity gene of tuber tubes. In application, the control of whether or not to form a potato tuber, formation time of tubers, and the regulation of the size of tubers may be controlled by regulating the protein function and expression level of IT1.

The nucleotide sequence of IT1 is not necessarily exactly the same in different potato materials. The present disclosure provides the nucleotide sequences of IT1 in other 44 potato materials obtained during the research process, and their CDS sequences respectively have 94.6% to 100% similarity (see Table 1 for details) to the sequence represented by SEQ ID NO: 1, in which the sequences that are not identical are represented by SEQ ID NOs: 10-39; their genomic sequences respectively have 91.8% to 100% similarity (see Table 1 for details) to the sequence represented by SEQ ID NO: 3, in which the sequences that are not identical are represented by SEQ ID NOs: 66-96.

In the present disclosure, a nucleotide sequence having less than 75% identity with the nucleotide sequence represented by SEQ ID NO: 1, but having the same function as the gene represented by SEQ ID NO: 1, such a sequence is also within the protection scope of the present disclosure.

In the present disclosure, genes having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with the nucleotide sequence represented by SEQ ID NO: 1 and having the same function are all within the protection scope of the present disclosure.

In some embodiments, genes having 60-70%, 75-85%, 76-86%, 77-87%, 78-88%, 79-89%, 80-90%, 81-91%, 82-92%, 83-93%, 84-94%, 85-95%, 86-96%, 87-97%, 88-98%, 89-99%, 90-95%, 91-96%, 92-97%, 93-98%, 94-99%, 95-100%, 85-90%, 86-91%, 87-92%, 88-93% or 89-94% identity with the nucleotide sequence represented by SEQ ID NO: 1 and having the same function are all within the protection scope of the present disclosure.

Particularly, the nucleotide sequence molecule may be DNA, such as cDNA, genomic DNA or recombinant DNA; the nucleotide sequence molecule may also be RNA, such as mRNA or hnRNA.

Those of ordinary skill in the art can easily mutate the nucleotide sequence encoding the protein according to the present disclosure by known methods, for example, directed evolution and point mutation. Those artificially modified nucleotides having 75% or higher identity with the nucleotide sequence isolated in the present disclosure, as long as they encode the protein, are derived from the nucleotide sequence of the present disclosure and are equivalent to the nucleotide sequence of the present disclosure.

Optionally, the polynucleotide according to the present disclosure further includes a promoter operably linked to the above nucleotide sequence, where the promoter is in a plant cell, preferably in stolons for initiating transcription of said nucleotide sequence.

Particularly, the promoter may be the nucleotide sequence represented by SEQ ID NO:9, or a polynucleotide having 75% or more identity with the nucleotide sequence represented by SEQ ID NO: 9.

In the present disclosure, genes having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with the nucleotide sequence represented by SEQ ID NO:9 and having the same function are all within the protection scope of the present disclosure.

In some embodiments, genes having 60-70%, 75-85%, 76-86%, 77-87%, 78-88%, 79-89%, 80-90%, 81-91%, 82-92%, 83-93%, 84-94%, 85-95%, 86-96%, 87-97%, 88-98%, 89-99%, 90-95%, 91-96%, 92-97%, 93-98%, 94-99%, 95-100%, 85-90%, 86-91%, 87-92%, 88-93% or 89-94% identity with the nucleotide sequence represented by SEQ ID NO:9 and having the same function are all within the protection scope of the present disclosure.

The present disclosure provides the promoter regions of IT1 nucleotide sequences in other 44 potato materials obtained during the research process, and they respectively have 90% to 100% identity with the nucleotide sequence represented by SEQ ID NO:9, in which the sequences that are not identical are represented by SEQ ID NOs: 97-131.

In the present disclosure, nucleotide sequences having less than 75% identity to the nucleotide sequence represented by SEQ ID NO: 9, but can initiating transcription of the above IT1 nucleotide sequence are also within the scope of the present disclosure.

The term “identity” as used herein refers to sequence similarity to a native nucleotide sequence or amino acid sequence. Identity may be assessed with the naked eye or with computer software. By computer software, the identity between two or more sequences may be expressed as a percentage (%), which may be used to evaluate the identity between related sequences.

In one aspect, the present disclosure also provides use of a protein in the regulation of development of a plant tuber, and the protein is at least one of the following sequences:
1) a protein represented by SEQ ID NO:2;
2) a fusion protein obtained by connecting a tag to the N-terminus and/or C-terminus of the protein represented by SEQ ID NO:2;
3) a protein with the same function obtained by substituting and/or deleting and/or adding one or several amino acid residues in the amino acid sequence represented by SEQ ID NO:2;
4) a protein that is 75% or more identical to an amino acid sequence represented by SEQ ID NO: 2 and has the same function.

In the present disclosure, proteins having less than 75% identity with the amino acid sequence represented by SEQ ID NO: 2, but having the same function as the protein represented by SEQ ID NO: 2, are also within the protection scope of the present disclosure.

In the present disclosure, proteins having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with the amino acid sequence represented by SEQ ID NO: 2 and having the same function are all within the protection scope of the present disclosure.

In some embodiments, proteins having 60-70%, 75-85%, 76-86%, 77-87%, 78-88%, 79-89%, 80-90%, 81-91%, 82-92%, 83-93%, 84-94%, 85-95%, 86-96%, 87-97%, 88-98%, 89-99%, 90-95%, 91-96%, 92-97%, 93-98%, 94-99%, 95-100%, 85-90%, 86-91%, 87-92%, 88-93% or 89-94% identity with the amino acid sequence represented by SEQ ID NO: 2 and having the same function are all within the protection scope of the present disclosure.

The present disclosure provides the amino acid sequences of IT1 obtained from 44 other potato materials during the research process, and they respectively have 92.9% to 100% similarity to the sequence represented by SEQ ID NO: 2 (see Table 1 for details), in which the sequences that are not identical are represented by SEQ ID NOs: 40-65.

TABLE 1
Comparison of the similarity between IT1 nucleotides, amino acid
sequences and promoter sequences in different potato materials

| | CDS | Amino acid | Genomic | Promoter |
|---|---|---|---|---|
| | sequence | sequence | sequence | sequence |
| Gene ID | similarity | similarity | similarity | similarity |
| Soltu.DM.06G025210.1 | 100 | 100 | 100 | 100 |
| (control) | | | | |
| St_A157_C06T002322.1 | 98.634 | 97.814 | 96.912 | 94.549 |
| St_C001_C00T015339.1 | 98.634 | 97.814 | 96.912 | 94.549 |
| St_C005_C00T061080.1 | 100 | 100 | 100 | 99.353 |
| St_C031_C00T046341.1 | 100 | 100 | 100 | 99.353 |
| St_C056_C00T016890.1 | 98.634 | 97.814 | 96.912 | 94.549 |
| St_C058_C00T041249.1 | 100 | 100 | 100 | 99.353 |
| St_C093_C00T005774.1 | 99.087 | 98.904 | 98.243 | 94.643 |
| St_C098_C00T057239.1 | 100 | 100 | 100 | 99.353 |
| St_C115_C00T013641.1 | 100 | 100 | 99.666 | 98.471 |
| St_C118_C00T022696.1 | 100 | 100 | 100 | 99.353 |
| St_C151_C00T059384.1 | 98.634 | 97.814 | 96.912 | 94.549 |
| St_C174_C00T005586.1 | 99.635 | 99.452 | 99.163 | 94.683 |
| St_C190_C00T013841.1 | 100 | 100 | 100 | 99.353 |
| St_C219_C00T010487.1 | 99.817 | 99.726 | 98.745 | 98.25 |
| St_C337_C00T002288.1 | 99.909 | 100 | 99.583 | 98.403 |
| St_C356_C00T038771.1 | 94.941 | 92.412 | 91.877 | 90.763 |
| St_C361_C00T031646.1 | 95.315 | 92.973 | 93.187 | 90.29 |
| St_C369_C00T029399.1 | 96.206 | 93.767 | 93.715 | 90.701 |
| St_C373_C00T027303.1 | 99.178 | 99.178 | 97.512 | 95.89 |
| St_C382_C00T009405.1 | 99.452 | 99.452 | 97.83 | 94.213 |
| St_C390_C00T010750.1 | 96.206 | 92.954 | 93.76 | 91.4 |
| St_C399_C00T037311.1 | 99.361 | 99.178 | 98.335 | 98.606 |
| St_C408_C00T002968.1 | 98.904 | 98.904 | 97.741 | 95.672 |
| St_C419_C00T037901.1 | 99.543 | 99.452 | 99.415 | 98.403 |
| St_C426_C00T036379.1 | 99.909 | 100 | 99.583 | 98.403 |
| St_C447_C00T007168.1 | 95.122 | 92.141 | 92.039 | 90.817 |
| St_C450_C00T001203.1 | 96.477 | 95.664 | 94.065 | 91.464 |
| St_C454_C00T016126.1 | 99.726 | 99.726 | 99.247 | 96.299 |
| St_C514_C00T044764.1 | 94.67 | 92.412 | 92.439 | 90.863 |
| St_C522_C00T011161.1 | 96.477 | 94.309 | 94.013 | 91.304 |
| St_C533_C00T013518.1 | 98.721 | 98.082 | 97.252 | 94.595 |
| St_C550_C00T004656.1 | 96.128 | 93.768 | 95.136 | 92.966 |
| St_C552_C00T018434.1 | 98.816 | 98.634 | 97.908 | 94.213 |
| St_C554_C00T003027.1 | 98.721 | 98.356 | 96.865 | 93.159 |
| St_C559_C00T012263.1 | 97.717 | 97.26 | 96.494 | 93.873 |
| St_C574_C00T045151.1 | 99.269 | 98.904 | 97.995 | 95.748 |
| St_C580_C00T019178.1 | 99.543 | 99.452 | 98.326 | 95.222 |
| St_C656_C00T014975.1 | 94.761 | 92.954 | 92.251 | 90.084 |
| St_C813_C00T054972.1 | 96.477 | 95.122 | 93.28 | 92.63 |
| St_C872_C00T000617.1 | 98.721 | 98.356 | 97.347 | 95.625 |
| St_E454_C06T002537.1 | 100 | 100 | 100 | 99.933 |
| St_E8669_C00T038840.1 | 100 | 100 | 100 | 99.353 |
| St_RH1015_ | 99.909 | 100 | 99.583 | 98.369 |
| C06T002364.1 | | | | |
| St_RH_C00T052849.1 | 98.539 | 98.082 | 97.995 | 94.893 |

In one aspect, the present disclosure also provides a biological material, which is any one of the following 1) to 9):
1) an expression cassette including the polynucleotide of claim 1;
2) a recombinant vector including the polynucleotide of claim 1;
3) a recombinant microorganism including the polynucleotide of claim 1;
4) a plant cell line including the polynucleotide of claim 1;
5) a knockout cassette knocking out the polynucleotide of claim 1;
6) a knockout vector knocking out the polynucleotide of claim 1;
7) a recombinant microorganism knocking out the polynucleotide of claim 1;
8) a plant cell line knocking out the polynucleotide of claim 1;
9) a plant protoplast, cell, or callus into which any one of 1)-3) or 5)-7) is introduced.

In the present disclosure, the plant cell line may or may not include propagation material.

In one aspect, the present disclosure also provides use of the above biological materials in the regulation of development of plant tuber.

In particular embodiments provided by the present disclosure, the regulation of the development of a plant tuber includes regulating one or more items including: whether a potato tuber is formed or not, formation time of tubers, number of the tubers or the size of tubers.

In particular embodiments provided by the present disclosure, the plant is a tuber plant.

In the embodiments provided by the present disclosure, the tuber plant is selected from potato, sweet potato, cassava, yam, taro, Helianthus tuberosus, Pinellia ternata or Stachys sieboldii. However, tuber plants are not limited to these. All tuber plants considered feasible by those skilled in the art are within the protection scope of the present disclosure.

In particular embodiments provided by the present disclosure, the tuber plant is potato.

The present disclosure also provides a method for the production of a tuber plant, where the above-mentioned polynucleotide or protein is overexpressed in a recipient plant to obtain a target plant; compared with a recipient plant, the target plant has the characteristics of earlier formation time of tubers, increased number of tubers, increased size of tubers, and/or increased yield of tubers.

In some embodiments, the formation time of tubers for a target plant is at least 30% earlier than a recipient plant. In some embodiments, the formation time of tubers for a target plant is at least 10%, at least 20%, at least 30%, at least 40%, at least 50%, at least 60%, at least 70%, at least 80%, at least 90%, or at least 100% earlier than a recipient plant. In some embodiments, the formation time of tubers for a target plant is at least 5 days, at least 10 days, at least 15 days, at least 20 days, at least 30 days, or at least 40 days, at least 50 days, or at least 60 days earlier than a recipient plant.

In some embodiments, the number of tubers, the size of tubers, or the yield of tubers for a target plant is at least 30% greater than a recipient plant. In some embodiments, the number of tubers, the size of tubers, or the yield of tubers for a target plant is at least 10%, at least 20%, at least 30%, at least 40%, at least 50%, at least 60%, at least 70%, at least 80%, at least 90%, or at least 100% greater than a recipient plant. In some embodiments, the number of tubers for a target plant is at least 1, at least 2, at least 3, at least 5, at least 10, or at least 20 greater than a recipient plant. In some embodiments, the size of tubers for a target plant is at least 5 g, at least 10 g, at least 15 g, at least 20 g, at least 30 g, at least 50 g, at least 80 g, or at least 100 g greater than a recipient plant. In some embodiments, the average tuber yield of a single target plant is at least 50 g, at least 100 g, at least 150 g, at least 200 g, at least 300 g, at least 500 g, at least 800 g, or at least 1000 g greater than a recipient plant.

In particular embodiments provided by the present disclosure, the methods of overexpression of the above-mentioned polynucleotide or protein in a receptor plant includes one or more techniques including promoter editing technology, codon optimization, utilization of a strong promoter, insertion of an intron, utilization of a viral vector, and fusion protein technology.

In some embodiments, the overexpression methods include the following optional methods.

A. Various methods of promoter editing technology are used to achieve overexpression. Promoter editing technology includes the following optional methods: a. small-scale changes in an endogenous promoter (deficiency or deletion of bases, but all operations are performed on the original endogenous promoter) (Rodriguez-Leal et al., 2017): CRISPR/Cas9 technology is used to design all sequences covering the promoter region, and finally, the changed promoter sequence will affect the binding ability of a transcription factor, and thus affect the expression of downstream genes; some promoters will include uORFs encoding small peptides, and during the transcription process of gene expression, the transcription of uORF will affect the transcription of downstream genes and thus affect the expression of downstream genes. Changing these uORFs by CRISPR/Cas9 technology may also change the expression of downstream genes. b. insertion of an element enhancing gene expression (such as an enhancer) into the original endogenous promoter by CRISPR/Cas technology so as to enhance gene expression. c. directly replacement of an endogenous promoter corresponding to a gene: firstly, replacing the endogenous promoter with a strong promoter (such as 35S, or a constitutively strong expression promoter) by knock-in; secondly, inducing chromosome inversion by CRISPR/Cas9 technology, thereby swapping the promoters of different genes and increasing the expression of target genes (Schwartz et al., 2020).

B. DNA sequence encoding a target gene is introduced into cells by transgenic methods. The following methods may be used alone or in combination: firstly, changing DNA sequence corresponding to an endogenous gene by codon optimization; secondly, using strong promoters (achieving overexpression in all tissues by strong constitutive promoters, and achieving overexpression in specific tissues by using tissue-specific promoters); thirdly, inserting certain introns which can enhance gene expression into target genes to enhance the expression of the target genes (Gallegos and Rose, 2019); fourthly, fusing certain tags or protein sequences that promote solubility or prevent degradation to the end of target genes to enhance the expression level of the target proteins or prevent their degradation.

C. Overexpression of the target gene is achieved by using a viral vector (such as DNA virus and RNA virus) (Torti et al., 2021). The virus will replicate in large quantities in plant cells, thereby greatly increasing the copy number of the target gene and achieving overexpression.

D. Other ways to achieve overexpression.

In one aspect, the present disclosure also provides a method for the production of a tuber plant, where the expression of the above-mentioned polynucleotide or protein in a recipient plant is inhibited, or the above protein in a recipient plant is inactivated, to obtain a target plant; compared with a recipient plant, the target plant has the characteristics of without formation of a tuber, or a delayed formation time of tubers, decreased number of tubers, decreased size of tubers, and/or decreased yield of tubers.

In some embodiments, the formation time of tubers for a target plant is at least 30% later than a recipient plant. In some embodiments, the tubers formation time of the target plant is at least 10%, at least 20%, at least 30%, at least 40%, at least 50%, at least 60%, at least 70%, at least 80%, at least 90%, or at least 100% later than a recipient plant. In some embodiments, the tubers formation time of the target plant is at least 5 days, at least 10 days, at least 15 days, at least 20 days, at least 30 days, at least 40 days, at least 50 days, or at least 60 days later than a recipient plant.

In some embodiments, the number of tubers, the size of tubers, or the yield of tubers for a target plant is at least 30% lower than a recipient plant. In some embodiments, the number of tubers, the size of tubers, or the yield of tubers for a target plant is at least 10%, at least 20%, at least 30%, at least 40%, at least 50%, at least 60%, at least 70%, at least 80%, at least 90%, or at least 100% lower than a recipient plant. In some embodiments, the number of tubers for a target plant is at least 1, at least 2, at least 3, at least 5, at least 10, or at least 20 lower than a recipient plant. In some embodiments, the size of tubers for a target plant is at least 5 g, at least 10 g, at least 15 g, at least 20 g, at least 30 g, at least 50 g, at least 80 g, or at least 100 g lower than a recipient plant. In some embodiments, the average tuber yield of a single target plant is at least 50 g, at least 100 g, at least 150 g, at least 200 g, at least 300 g, at least 500 g, at least 800 g, or at least 1000 g lower than a recipient plant.

In particular embodiments provided by the present disclosure, methods for inhibiting the expression of the above-mentioned polynucleotides or proteins in recipient plants and inactivating the above proteins in recipient plants include one or more techniques including: CRISPR/Cas9 gene editing technology, TALEN technology, and T-DNA insertion, EMS mutagenesis, and ZFN technology. Other methods for inhibiting expression or inactivating may also be chosen.

In one aspect, the present disclosure also provides a tuber plant, the plant parts, tubers or tuber parts thereof, or the plant cells, pollens, or seeds thereof, where it is one of the following:
1) a plant grown from the above mentioned plant cell line, the plant protoplast, cell or callus, or the plant parts, tubers or tuber parts thereof, or the plant cells, pollens or seeds thereof; or a plant obtained by any one of the above mentioned production methods, the plant parts, tubers or tuber parts thereof, or the plant cells, pollens or seeds thereof;
2) progeny obtained by selfing of the plants from the above 1), and the plants formed by the growth of said progeny, the plant parts, tubers or tuber parts thereof, or the plant cells, pollens or seeds thereof;
3) progeny obtained by crossing a plant from the above 1) with another variety, and the plants formed by the growth of said progeny, the plant parts, tubers or tuber parts thereof, or the plant cells, pollens or seeds thereof.

In one aspect, the present disclosure also provides food or feed prepared from the above-mentioned plant, the plant parts, tubers or tuber parts thereof, or the plant cells, pollens or seeds thereof.

In one aspect, the present disclosure also provides a method for manufacturing a commercial plant product, which includes obtaining the above-mentioned plant, the plant parts, tubers or tuber parts thereof to manufacture the commercial plant product, where the plant product is selected from the group consisting of: fresh tuber material, frozen tuber material, dehydrated tuber material, tuber liquid, tuber strips, tuber slices, tuber granules and tuber powder.

In one aspect, the present disclosure also provides the use of the above biological materials in regulating development of potato tubers.

In particular embodiments provided by the present disclosure, the above-mentioned polynucleotides, proteins, or biological materials are used to regulate development of potato tubers. However, the plants are not limited to potatoes, and other plants whose tuber development may be regulated through the technical solution of the present disclosure are also within the protection scope of the present disclosure.

Further, the potatoes include diploid potatoes, triploid potatoes, tetraploid potatoes, or other polyploid potatoes.

In some embodiments, the potato is selected from the group consisting of: potato lines NCIMB 41663, NCIMB 41664, NCIMB 41665, NCIMB 41765, NCIMB 43636, Favorita, Qingshu No. 9, Kexin No. 1, Jizhangshu No. 12, Mila, Weiyu No. 3, Longshu No. 7, Weiyu No. 5, E (Hubei) Potato No. 5, Atlantic, Lishu No. 6, Longshu No. 10, Longshu No. 3, Zhongshu No. 5, Jinshu No. 16, Hezuo 88, Zaodabai, Jizhangshu No. 8, Dongnong 303, Zhuangshu No. 3, Zhongshu No. 3, Xingjia No. 2, Xisen No. 6 and Hui-2.

In the embodiments provided by the present disclosure, the regulation of development of potato tubers includes regulating one or more items including: whether a potato tuber is formed or not, formation time of potato tubers, the number of the potato tubers or the size of potato tubers.

In another aspect, the present disclosure also provides a method for the production of potato plants, where the above-mentioned polynucleotide or the protein is overexpressed in a recipient plant to obtain a target plant; compared with a recipient plant, the target plant has the characteristics of earlier formation time of potato tubers, increased number of potato tubers, increased size of potato tubers, and/or increased yield of potato tubers.

In some embodiments, the overexpression methods are the same as described above, and any one or more of the methods, or other methods that can achieve the purpose of overexpression may be selected.

In some embodiments, the formation time of potato tubers of a target plant is at least 30% earlier than a recipient plant. In some embodiments, the formation time of potato tubers of a target plant is at least 10%, at least 20%, at least 30%, at least 40%, at least 50%, at least 60%, at least 70%, at least 80%, at least 90%, or at least 100% earlier than a recipient plant. In some embodiments, the formation time of potato tubers of a target plant is at least 5 days, at least 10 days, at least 15 days, at least 20 days, at least 30 days, or at least 40 days, at least 50 days, or at least 60 days earlier than a recipient plant.

In some embodiments, the number of potato tubers, the size of potato tubers, or the yield of potato tubers for a target plant is at least 30% greater than a recipient plant. In some embodiments, the number of potato tubers, the size of potato tubers, or the yield of potato tubers for a target plant is at least 10%, at least 20%, at least 30%, at least 40%, at least 50%, at least 60%, at least 70%, at least 80%, at least 90%, or at least 100% greater than a recipient plant. In some embodiments, the number of potato tubers for a target plant is at least 1, at least 2, at least 3, at least 5, at least 10, or at least 20 greater than a recipient plant. In some embodiments, the size of potato tubers for a target plant is at least 5 g, at least 10 g, at least 15 g, at least 20 g, at least 30 g, at least 50 g, at least 80 g, or at least 100 g greater than a recipient plant. In some embodiments, the average tuber yield of a single target plant is at least 50 g, at least 100 g, at least 150 g, at least 200 g, at least 300 g, at least 500 g, at least 800 g, or at least 1000 g greater than a recipient plant.

In one aspect, the present disclosure also provides another method for the production of potato plants, where the expression of the above mentioned polynucleotide or protein in a recipient plant are inhibited, or the above protein in a recipient plant is inactivated, so as to obtain a target plant; compared with a recipient plant, the target plant does not form potato tubers, or has delayed formation time of potato tubers, reduced number of potato tubers, smaller potato tubers, and/or reduced yield of potato tubers.

In some embodiments, the methods for inhibiting the expression of the above polynucleotide or protein in a recipient plant and inactivating the protein in a recipient plant are as described above, and any one or more of the methods, or other methods that can achieve inhibition of expression or inactivation may be selected.

In a particular embodiment provided by the present disclosure, the method for inhibiting the expression of the above polynucleotide or protein in a recipient plant and inactivating the protein in a recipient plant is CRISPR/Cas9 gene editing technology. The specific steps of CRISPR/Cas9 gene editing technology are as follows: using a gene as a target to design a sgRNA sequence based on CRISPR/Cas9, linking a DNA fragment including the coding sgRNA sequence to a vector carrying CRISPR/Cas9, transforming a recipient plant to obtain a target plant which lacks the function of said gene.

In some embodiments, the formation time of potato tubers of a target plant is at least 30% later than a recipient plant. In some embodiments, the formation time of potato tubers of a target plant is at least 10%, at least 20%, at least 30%, at least 40%, at least 50%, at least 60%, at least 70%, at least 80%, at least 90%, or at least 100% later than a recipient plant. In some embodiments, the formation time of potato tubers of a target plant is at least 5 days, at least 10 days, at least 15 days, at least 20 days, at least 30 days, at least 40 days, at least 50 days, or at least 60 days later than a recipient plant.

In some embodiments, the number of potato tubers, the size of potato tubers, or the yield of potato tubers for a target plant is at least 30% lower than a recipient plant. In some embodiments, the number of potato tubers, the size of potato tubers, or the yield of potato tubers for a target plant is at least 10%, at least 20%, at least 30%, at least 40%, at least 50%, at least 60%, at least 70%, at least 80%, at least 90%, or at least 100% lower than a recipient plant. In some embodiments, the number of potato tubers for a target plant is at least 1, at least 2, at least 3, at least 5, at least 10, or at least 20 lower than a recipient plant. In some embodiments, the size of potato tubers for a target plant is at least 5 g, at least 10 g, at least 15 g, at least 20 g, at least 30 g, at least 50 g, at least 80 g, or at least 100 g lower than a recipient plant. In some embodiments, the average tuber yield of a single target plant is at least 50 g, at least 100 g, at least 150 g, at least 200 g, at least 300 g, at least 500 g, at least 800 g, or at least 1000 g lower than a recipient plant.

In one aspect, the present disclosure also provides a potato plant, the plant parts, tubers or tuber parts thereof, or the plant cells, pollens or seeds thereof, where it is one of the following:
1) a potato plant grown from the above mentioned plant cell line, the plant protoplast, cell or callus, or the plant parts, tubers or tuber parts thereof, or the plant cells, pollens or seeds thereof; or a potato plant obtained by any one of the above mentioned production methods, the plant parts, tubers or tuber parts thereof, or the plant cells, pollens or seeds thereof;
2) progeny obtained by selfing of the plants from the above 1), and the potato plants formed by the growth of said progeny, the plant parts, tubers or tuber parts thereof, or the plant cells, pollens or seeds thereof;
3) progeny obtained by crossing a plant from the above 1) with another variety, and the potato plants formed by the growth of said progeny, the plant parts, tubers or tuber parts thereof, or the plant cells, pollens or seeds thereof.

In one aspect, the present disclosure also provides food or feed prepared from the above-mentioned potato plant, the plant parts, tubers or tuber parts thereof, or the plant cells, pollens or seeds thereof.

In one aspect, the present disclosure also provides a method for manufacturing a commercial plant product, which includes obtaining the above-mentioned potato plant, the plant parts, tubers or tuber parts thereof to manufacture the commercial plant product, where the plant product is selected from the group consisting of: fresh potatoes, frozen potatoes, dehydrated potatoes, potato juice, potato strips, potato slices, potato granules, potato powder, and potato starch.

Compared with the prior art, the technical effects of the present disclosure are as follows.

Prior to the present disclosure, research progress related to development of potato tubers was mostly focused on the analysis of the molecular mechanisms of the comprehensive effects of plant endogenous signals and the external environment.

For example, plant endogenous hormones are important signaling factors that regulate tuber formation. GA-20 oxidase (GA200X) is a dioxygenase that can catalyze multi-step oxidation reactions and ultimately promote the synthesis of gibberellins. In 1999, Carrera et al. first cloned three gibberellin oxidase genes in potatoes (StGA20OX1-3), their research showed that overexpression of these genes in potatoes can delay potato formation, while inhibition of their expression can accelerate potato formation and increase yield (Carrera, E., Bou, J., García-Martinez, J. L., and Prat, S. (2000). Plant J. Cell Mol. Biol. 22:247-256; Carrera, E., Jackson, S. D., and Prat, S. (1999). Plant Physiol. 119:765-774). Further research by Kloosterman et al. showed that the StGA20OX1 gene regulates potato tuber formation by regulating the content of GA in the sub-apical region of stolons (Kloosterman et al., 2007). GA-2 oxidase (GA20X) is a key enzyme in the third stage of the gibberellin synthesis pathway. It can convert active gibberellin into inactive gibberellin to inhibit the synthesis of gibberellin. Overexpression of StGA2OX1 in potato plants will inhibit the growth of stolons and promote early tuber formation, while inhibition of its expression will delay tuber formation (Kloosterman et al., 2007). Therefore, it may be inferred that GA can promote the growth of stolons and inhibit tuber formation during development of potato tubers. Cytokinin oxidase/dehydrogenase (CKX) is a flavoenzyme that can degrade cytokinins into adenine/adenosine. Overexpression of CKX1 in potato plants will reduce the number of potato tubers per plant, thereby affecting yield (Hartmann et al., 2011). TLOG1 is a cytokinin synthesis gene in tomato. Ectopic expression of this gene in tomato can relieve the inhibition of tuber formation by the axillary meristem of tomato, thereby forming tiny tubers at the base of the axillary buds (Eviatar-Ribak et al., 2013; Abelenda and Prat, 2013). These results indicate that cytokinins are likely to be broad-spectrum regulators of storage organ formation in plants. In addition, in addition to gibberellins and cytokinins, it was reported that hormones such as auxin, strigolactone, and abscisic acid also play an important role in potato tuber formation (Navarro et al., 2015).

In addition, the external growth environment also plays an important role in the formation of potato tubers; particularly, photoperiod has the most obvious impact on tuber formation. Potatoes are native to the western foothills of the central Andes Mountains in South America, particularly the regions of Peru and Bolivia bordering the Pacific Ocean. In their origin places, short days can induce the formation of potato tubers, while long days inhibit tuber formation. Photoperiod is a key environmental factor for the establishment of potato tuber morphology. Research results in recent years have shown that, some regulatory elements involved in the flowering pathway, such as phytochrome B (PHYB), CONSTANS (CO), FLOWERING LOCUS T (FT), and CDF transcription factors also play an important role in the potato tuber formation pathway.

PHYB is an important photoreceptor that regulates development of potato tubers. Down-regulation of its expression will significantly enhance the ability of a potato plant to form potato tubers under long-day conditions. PHYB is mainly expressed in leaves, and a grafted potato plant obtained by using the above-ground parts of a potato plant with significantly down-regulated PHYB expression as scion and using a wild plant as rootstock can still quickly form tubers under long-day conditions, indicating that PHYB can sense light signals in leaves and inhibit tuber formation under long-day conditions (Jackson et al., 1998). CONSTANS (CO) is a key factor in regulating flowering in Arabidopsis. Overexpression of AtCO in potatoes can delay tuber formation in potatoes grown under short days by more than 7 weeks. StCO has a similar function to AtCO, and overexpression of StCO in potatoes can also delay tuber formation. Further grafting experimental results showed that a grafted potato plant obtained by grafting the upper part of a AtCO or StCO overexpressing plant to a wild-type potato plant can significantly delay the formation of tuber; conversely, it does not affect the development of tuber. It is indicated that CONSTANS (CO) is expressed in leaves to negatively regulate the formation of potato tubers (Martínez-García et al., 2002; González-Schain et al., 2012). CDFs protein is a member of the DOF transcription factor family and is a type of transcription repressor that can directly bind to the promoter regions of CO and FT in Arabidopsis to inhibit plant flowering. StCDF1 is the homologous protein of AtCDFs in potato and can also regulates the expression of StCO, and its protein content is jointly regulated by the LOV blue light receptor protein StFKF1 and the circadian clock core protein StGI. In potatoes, when the domain where StCDF1 interacts with StFKF1 and StGI is mutated, the protein amount of StCDF1 can be accumulated, thereby promoting tubers to be formed early (Kloosterman et al., 2013). Early research found that grafting a flowering tobacco stem segment onto a potato plant can effectively induce the formation of potato tubers, indicating that tuberigen inducing the formation of potato tubers may exist in the leaves or stem segments of a flowering plant (Chailakhyan et al., 1981). The FLOWERING LOCUS T (FT) protein of Arabidopsis and the Hd3a protein of rice were identified as a type of florigen inducing plant flowering. Overexpression of Hd3a in a potato plant can induce normal formation of potato tubers under long-day conditions, indicating that potato tuberigen is probably also a FT-type protein. There are four FT-type genes StSP6A, StSP5G, StSP5G-like and StSP3D in potatoes. Particularly, StSP6A is mainly expressed in leaves under short-day conditions, and its overexpression can induce a potato plant to produce tubers normally under long-day conditions, and inhibition of its expression will delay tuber formation. Grafting experiments have shown that, StSP6A may be synthesized in leaves and transported to stolons over long distances. These results prove that StSP6A is a tuberigen in potatoes. In addition, studies have found that StSP3D can independently regulate plant flowering, while StSP5G can inhibit tuber formation by inhibiting the expression of StSP6A (Navarro et al., 2011; Abelenda et al., 2016).

However, in the study of genes regulating potato tuber formation, it has not been reported that mutation of a certain gene can prevent potato stolons from further developing into tubers.

The present disclosure found that IT1 is a key formation gene regulating the formation of potato tubers. In application, it may be used to control whether a potato tuber is formed or not, the formation time of potato tubers, the number of the potato tubers or the size of potato tubers by regulating the protein function and expression level of IT1. Furthermore, as for other tuber plants, it is also possible to control whether to form tubers or not, the formation time of tubers, the size of tubers, the number of tubers, and the yield of tubers by regulating the protein function and expression level of IT1.

### BRIEF DESCRIPTION OF THE DRAWINGS

FIG. 1 shows the genotype of the homozygous mutant line of the potato IT1 gene; compared with a wild-type line, the mutant line has a 1 bp base inserted, and is a loss-of-function mutant; “1 bp insertion” in FIG. 1 means “insertion of 1 bp base”.

FIG. 2-1 shows the result of a wild-type plant (WT) in phenotypic identification of the homozygous mutant line of the potato IT1 gene; the scale bar is 10 cm.

FIG. 2-2 shows the result of a IT1 gene homozygous mutant line (it1) in phenotypic identification of the homozygous mutant line of the potato IT1 gene; compared with a wild-type plant (WT), the stolons did not further expand and develop into tubers after the top was hooked, but grew upward and developed into a new plant, as shown in the position indicated by Δ; the scale bar is 10 cm.

FIG. 2-3 shows the phenotypic identification results of the homozygous mutant line of the potato IT1 gene; the scale bar is 5 cm.

### DETAILED DESCRIPTION OF THE EMBODIMENTS

The present disclosure discloses the use of polynucleotides, proteins, and biological materials in regulating the development of plant tubers, as well as related products and production methods. A person skilled in the art can learn from the disclosure of this application and appropriately improve the process parameters for implementation. It should be noted that all similar substitutions and modifications are obvious to those skilled in the art, and they are deemed to be included in the present disclosure. The methods and use according to the present disclosure have been described through preferred embodiments. Relevant persons can obviously make modifications or appropriate changes and combinations to the methods and uses described herein without departing from the content, spirit and scope of the present disclosure to achieve and apply the technology of this disclosure.

### Terminology Explanations

N-terminus: refers to the amino terminus of a peptide chain or protein.

C-terminus: refers to the carboxyl terminus of a peptide chain or protein.

cDNA: the full name is complementary DNA, which is a kind of complementary deoxyribonucleic acid.

CRISPR/Cas9 system: CRISPR/Cas9 is an adaptive immune defense system formed by bacteria and archaea during the long-term evolution process. It may be used to fight against invading viruses and foreign DNA. The CRISPR/Cas9 system provides immunity by integrating fragments of invading phage and plasmid DNA into CRISPR and using the corresponding CRISPR RNAS (crRNAs) to direct the degradation of homologous sequences. The working principle of this system is that crRNA (CRISPR-derived RNA) combines with tracrRNA (trans-activating RNA) by base pairing to form a tracrRNA/crRNA complex, and this complex guides the nuclease Cas9 protein to cut double-stranded DNA at a target site of the sequence which is paired with crRNA. By artificially designing these two types of RNA, sgRNA (single-guide RNA) with guiding function may be formed by transformation, and it is enough to guide Cas9 to cut DNA at a specific location.

TALEN: transcription activator-like effector nuclease.

T-DNA: (Transfer DNA), also known as triple helix DNA, is a special deoxyribonucleotide structure formed by three strands of ssDNA rotating helices.

EMS mutagenesis: is an artificial chemical mutagenesis technology. EMS is ethyl methyl sulfonate, which is the most commonly used type of alkylating agent mutagenesis and has a high mutation rate.

ZFN: zinc-finger nuclease (ZFN), also known as zinc finger protein nuclease (ZFPN), is a type of artificially synthetic restriction endonuclease, which is formed by fusion of a zinc finger DNA-binding domain and the DNA cleavage domain of a restriction endonuclease. Researchers can modify the zinc finger DNA-binding domain of ZFN to target different DNA sequences, so that ZFN can bind to a target sequence in a complex genome and perform specific cleavage using the DNA cleavage domain. At present, ZFN technology has been widely used in targeted gene mutations in a large number of species including fruit flies, zebrafish, frogs, rats/mouse, and cattle. New species with modified genetic backgrounds may be generated by artificially modifying genomic information.

TCP transcription factors: belong to a transcription family unique to higher plants, and they are named after the acronym of three isolated members: Teosinte branched 1 (TB1), Cycloidea (CYC) and Proliferating cell factors (PCFs).

sgRNA (small guide RNA): is a guide RNA, which guides the insertion or deletion of uridine residues into kinetoplastids during the RNA editing process. It is a small non-coding RNA that may be paired with pre-mRNA. gRNA edits RNA molecules, has approximately 60-80 nucleotides in length, and is transcribed by individual genes.

WT: is an abbreviation of wild type.

CDS sequence: CDS is an abbreviation of Coding sequence. DNA is transcribed into mRNA, and the mRNA is translated into protein after splicing and other processing. The so-called CDS is a DNA sequence that corresponds one-to-one to a protein sequence, and the sequence does not include other sequences that do not correspond to the protein, without consideration of sequence changes during the process such as mRNA processing.

Sanger sequencing: the Sanger method is to start at a fixed point based on the nucleotide, and to end randomly at a specific base, then fluorescently labeling behind each base, resulting in a series of four sets of nucleotides having different lengths ended with A, T, C, or G. Electrophoresis is then performed on a urea-denatured PAGE gel for detection to obtain a visible DNA base sequence.

The experimental methods in the following Examples are all conventional methods unless otherwise specified.

The biological materials, sequences, reagents or instruments used in the Examples of the present disclosure are commercially available.

Note: the IT1 gene in this disclosure is an abbreviation of the Identity of Tuber I gene, and includes Soltu. DM.06G025210.1 gene and/or its homologous genes.

IT1 or IT1 stands for a gene or protein, and it may be determined based on context.

The present disclosure will be further described below in conjunction with the Examples.

### Example 1: Knocking Out the IT1 Gene by CRISPR/Cas9 System

IT1 gene in potato was knocked out by CRISPR/Cas9 system to obtain a potato mutant with IT1 gene knockout. The specific steps are as follows.

### Step 1: Selection of sgRNA Sequence

A target site sequence with a length of 20 bp was designed on IT1 gene.

Sequence 1 (IT1 gene CDS) is represented by SEQ ID NO: 1, sequence 2 (protein) is represented by SEQ ID NO: 2, and sequence 3 (genome) is represented by SEQ ID NO: 3.

The target sites are located at positions 308-327 of sequence 1 (CDS sequence), and positions 720-739 of sequence 3 (genome sequence). The sequence of target site 1 is GAATTAGTATTATTAGTGGA (sequence 4, SEQ ID NO: 4).

SgRNA sequence designed according to the target site is as follows:

(Sequence 5, SEQ ID NO: 5)
GUUUUAGAGCUAGAAAUAGCAAGUUAAAAUAAGGCUAGUCCGUUAUCAA
CUUGAAAAAGUGGCACCGAGUCGGUGC

The coding DNA molecule of this sgRNA is as follows:

(Sequence 6, SEQ ID NO: 6)
GTTTTAGAGCTAGAAATAGCAAGTTAAAATAAGGCTAGTCCGTTATCAA
CTTGAAAAAGTGGCACCGAGTCGGTGC.

### Step 2: Construction of CRISPR/Cas9 Vector

The original vector includes the sgRNA sequence, and the sequence of target site 1 in step 1 is further inserted into the vector to obtain a CRISPR/Cas9 vector.

### Step 3: Obtaining Transgenic Plants

The CRISPR/Cas9 vector obtained in step 2 was transferred into competent cells of Arobacterium tumefaciens EHA105 by heat shock transformation (competent cells of Arobacterium tumefaciens EHA105 are from Shanghai Weidi Biotechnology Co., Ltd., and are available to the public through purchase) to obtain the recombinant line EHA105/CRISPR/Cas9.

The recombinant line EHA105/CRISPR/Cas9 was then used to transform potatoes by Agrobacterium infection (the recombinant Agrobacterium was propagated at 28° C., and the amplified Arobacterium tumefaciens solution was used to infect potatoes), and genetically modified T₀ generation potato plants were obtained after kanamycin resistance screening.

### Example 2: Identification of Transgenic Plants with Mutated IT1 Gene

The leaves of the genetically modified T₀ generation potato plants obtained from step 3 in Example 1 were collected, and the genomic DNA was extracted as a template. The following primer pairs were used to perform PCR amplification to obtain PCR amplification products of different potato lines.

The sequences of detection primers for IT1 mutation sequence are as follows:

IT1-test-F:
(Sequence 7, SEQ ID NO: 7)
AGCTACAGCTACTGCGCAAA
IT1-test-R:
(Sequence 8, SEQ ID NO: 8)
AGTAGTGGGGGTATTGCAGC

The PCR amplification products of different lines were subjected to Sanger sequencing, and the sequencing results were compared with the wild-type IT1 gene. The IT1 genotypes were identified separately according to the following principles.

When a sequence has bimodal characteristics from the target site sequence, the genotype of this line is a heterozygous genotype (IT/gene is mutated on one of the two homologous chromosomes, and IT1 gene on the other chromosome is not mutated), and this line is a heterozygous mutant line of genetically modified T₀ generation potato plants.

When a sequence has bimodal characteristics from the target site sequence, and the IT1 gene in both homologous chromosomes is mutated, then this line is a biallelic mutant line of genetically modified T₀ generation potato plants.

A sequence has specific single peak characteristics from the target site sequence, when it is the same as the IT1 gene sequence of wild-type potato, then the genotype of this line is wild-type, that is, the IT1 gene sequence has not mutated; and when it is not the same as the IT1 gene sequence of wild-type potato, then the genotype of this line is a homozygous genotype (IT1 genes on both homologous chromosomes are mutated), and this line is a homozygous mutant line of genetically modified T₀ generation potato plants.

In this example, the IT1 gene homozygous mutant line of genetically modified T₀ generation potato plants was identified (as shown in FIG. 1), which was used to identify the growth phenotypes of the following potato tubers.

### Example 3: Potato Mutants with IT1 Gene Knockout have Abnormal Growth and Development of Potato Tubers

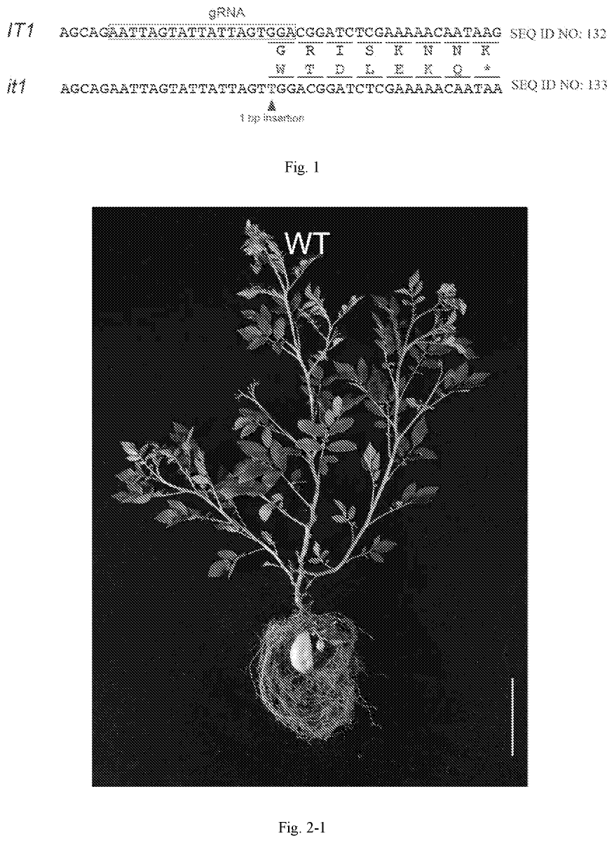

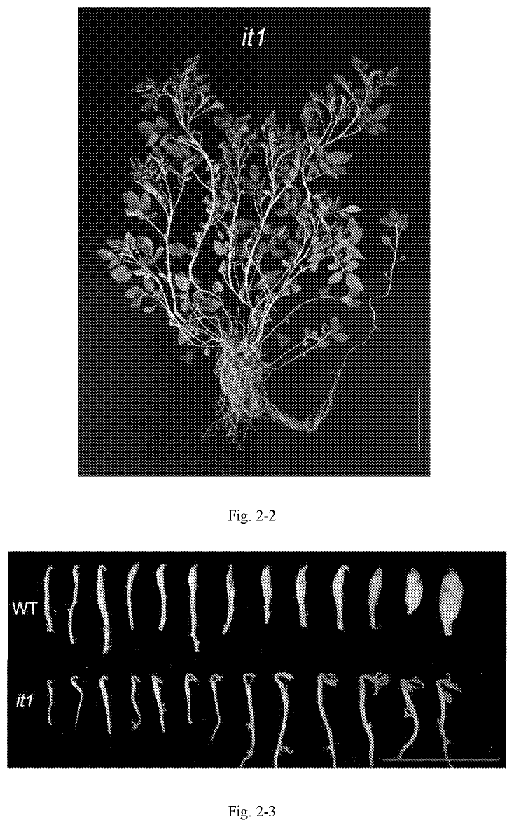

The IT1 gene homozygous mutant line of genetically modified T₀ generation potato plants obtained in Example 2 was planted in the greenhouse of the Nankou Pilot Base of the Chinese Academy of Agricultural Sciences in the autumn of 2021. After systematic identification of the phenotype of potato tubers formed by the mutants, it was found that the stolons of the IT1 homozygous mutant did not further expand and develop into tubers after the top was hooked, but grew upward and developed into the phenotype of a new plant, indicating that IT1 plays a key role in regulating the formation of potato tubers (see FIG. 2-1 to FIG. 2-3).

Sequences 1-3 in the above Examples are as follows:

Sequence 1 (CDS):
ATGTATCCTCCAAGCAACAATAACTGCAACTACAGCCCAATTTTGTCTT
CTTTCATATGCCAAAATATTCCATCTTCTCCTTGTATGCAATACGAACA
CGAACTATACTTTCAAAACTTCAATCATGATGACCAATATTATTTTCAA
CTACAGCAACAAGTTCCCTTGATAGATGACTTGAGTCCTCACGTCTTAG
CTGACAGCTGCACTGAGACTGTTACTAAGCCTTCAAATTGCAATCACGT
ACTAGAAGGAATGGAAGAAGGCCGAGGCGGAAACAAAGGAGATGATGTT
GTTATGAGTAGCAGAATTAGTATTATTAGTGGACGGATCTCGAAAAACA
ATAAGAGATCTTCCAATAAGGATCGACACAGCAAGATCAACACGGCTCG
TGGTCCAAGAGATCGAAGGATGAGACTTTCACTTGATGCTGCTCGCAAG
TTTTTCCGTTTGCAGGACTTATTGGGATTTGATAAGGCCAGCAAAACTG
TAGAATGGTTGCTTACTCAATCGGATTCCGCAATTGAAGAGCTCGTCGC
CGTTAAAGGCAATGATGCTCAGGTTCCTCAGCAAACTAGCTGCAATACC
CCCACTACTACTACTGGAATTGGTGCAATTTGTGCATCTAATTCTATTT
CTGAGTCATGTGAAGTTATATCAGGAACTGATGAAACTTCCTCTAATGA
CAAAAACAAGGAAACTACTGCTAAAGATGAGAAGGAGAAAAAGAAGAAG
CCGGTTAACACAGCTCGTAGACCTGCGTTTGAACCTCTTACAAAGGAAT
CAAGGAATCAAGCAAGAGCCAGGGCTAGAGAGAGAACAAAAACAAAGAA
AATGAGCCAAGTTGGAAAATCCAAATCCCCAGCTCATGATTTGAACCCT
TCAGGATCTCGGAGGCCGGCTAATAGAACTTGTGAAGAACCTGGAACAC
ATGAACAACACACCTTCCATCATGTTGATGACAGTAGTTTTGTGGTTAA
TGGAAATTGGAATCCATTTACAATCTTCACTTCTCATGAACAATATGCT
GGAATTTCCAATGAGCATCAATTAGTTACAGACTTGCAATTTTATGGAA
AGCTGTGGGAAAGCTAG
Sequence 2 (protein):
MYPPSNNNCNYSPILSSFICQNIPSSPCMQYEHELYFQNFNHDDQYYFQ
LQQQVPLIDDLSPHVLADSCTETVTKPSNCNHVLEGMEEGRGGNKGDDV
VMSSRISIISGRISKNNKRSSNKDRHSKINTARGPRDRRMRLSLDAARK
FFRLQDLLGFDKASKTVEWLLTQSDSAIEELVAVKGNDAQVPQQTSCNT
PTTTTGIGAICASNSISESCEVISGTDETSSNDKNKETTAKDEKEKKKK
PVNTARRPAFEPLTKESRNQARARARERTKTKKMSQVGKSKSPAHDLNP
SGSRRPANRTCEEPGTHEQHTFHHVDDSSFVVNGNWNPFTIFTSHEQYA
GISNEHQLVTDLQFYGKLWES
Sequence 3 (genome):
CATGCCTGTAGCTTGATGCTTAGACGGGTGCACACGCACTCTCTCACTC
ACACAGCTAGAATATATATATATATATATATATATATTCATAGTTAGCA
GAAGTACTTATCATATACCAAAAACCACACAAATACATTGTATCAAGTG
CTGTCATACTCAAGCAAAAGAAAGAAAAGAACAAGATATAGTACTACTG
TTTTCATCACCATTTTGGTCAATCATGATGATTCTGAACAAAGATATAG
TACTAGCTAGGTAGAAAATAAATCTACCAACTTTAATTTTCTTCTTATT
GCAGCTAGCTTGCTTAATTAGCAGCAAAACTCAAAAGAGGTTTTAGCTG
TGTTTATACTGTCTTTCTCAAGATCTAGACCCACCACTTAGACCATCTC
AAGCTACAGCTACTGCGCAAATGTATCCTCCAAGCAACAATAACTGCAA
CTACAGCCCAATTTTGTCTTCTTTCATATGCCAAAATATTCCATCTTCT
CCTTGTATGCAATACGAACACGAACTATACTTTCAAAACTTCAATCATG
ATGACCAATATTATTTTCAACTACAGCAACAAGTTCCCTTGATAGATGA
CTTGAGTCCTCACGTCTTAGCTGACAGCTGCACTGAGACTGTTACTAAG
CCTTCAAATTGCAATCACGTACTAGAAGGAATGGAAGAAGGCCGAGGCG
GAAACAAAGGAGATGATGTTGTTATGAGTAGCAGAATTAGTATTATTAG
TGGACGGATCTCGAAAAACAATAAGAGATCTTCCAATAAGGATCGACAC
AGCAAGATCAACACGGCTCGTGGTCCAAGAGATCGAAGGATGAGACTTT
CACTTGATGCTGCTCGCAAGTTTTTCCGTTTGCAGGACTTATTGGGATT
TGATAAGGCCAGCAAAACTGTAGAATGGTTGCTTACTCAATCGGATTCC
GCAATTGAAGAGCTCGTCGCCGTTAAAGGCAATGATGCTCAGGTTCCTC
AGCAAACTAGCTGCAATACCCCCACTACTACTACTGGAATTGGTGCAAT
TTGTGCATCTAATTCTATTTCTGAGTCATGTGAAGTTATATCAGGAACT
GATGAAACTTCCTCTAATGACAAAAACAAGGAAACTACTGCTAAAGATG
AGAAGGAGAAAAAGAAGAAGCCGGTTAACACAGCTCGTAGACCTGCGTT
TGAACCTCTTACAAAGGAATCAAGGAATCAAGCAAGAGCCAGGGCTAGA
GAGAGAACAAAAACAAAGAAAATGAGCCAAGTTGGAAAATCCAAATCCC
CAGCTCATGATTTGAACCCTTCAGGATCTCGGAGGCCGGCTAATAGAAC
TTGTGAAGAACCTGGAACACATGAACAACACACCTTCCATCATGTTGAT
GACAGTAGTTTTGTGGTTAATGGAAATTGGAATCCATTTACAATCTTCA
CTTCTCATGAACAATATGCTGGAATTTCCAATGAGGTGAGGGCTTCAAT
AATTAATTAAATCCAGTAGATTTCTATTTATATATATATATATATATTC
TTATCAGCTTCTAAAAAAAATCCTTATTTCTCTGCAGCATCAATTAGTT
ACAGACTTGCAATTTTATGGAAAGCTGTGGGAAAGCTAGGGCAAGGAAA
TTCGAAGCGGACAAAGTTCCTTCTTCATTTTGTGCTTACTCGGCCGAGC
AGAAGAACGTTTCCGGCCTGTATCTGTTGGTAAATGTAACATACATTTC
TACTTTTTAAGTGTGTTTTTCTTTTGTGAAAGAACTTCATGTGCAGATA
GCCTATGTTTTTTTTTTGAATAAAACTATGATGGGGTTCATTAAGCAAG
CGACATATTCGGG

The above descriptions are only the preferred embodiments of the present disclosure. It should be pointed out that, those skilled in the art can also make several improvements and modifications without departing from the principles of the present disclosure. These improvements and modifications should also be regarded as falling into the protection scope of the present disclosure.

What is claimed is:

1. A tuber plant or part thereof, comprising a sequence encoding a polypeptide comprising a sequence having at least 92% sequence identity to SEQ ID NO: 2, wherein the polypeptide is inhibited-expressed, loss-function, or overexpressed, and the development of plant tuber is changed.

2. The tuber plant or part thereof of claim 1, wherein the polypeptide comprises a sequence shown in SEQ ID NO: 2 or SEQ ID NO: 40˜SEQ ID NO: 65.

3. The tuber plant or part thereof of claim 1, wherein the sequence comprises a sequence having at least 91% sequence identity to SEQ ID NO: 3.

4. The tuber plant or part thereof of claim 3, wherein the sequence comprises a sequence shown in SEQ ID NO: 3 or SEQ ID NO: 66˜SEQ ID NO: 96.

5. The tuber plant or part thereof of claim 1, wherein the sequence comprises a sequence having at least 94% sequence identity to SEQ ID NO: 1.

6. The tuber plant or part thereof of claim 5, wherein the sequence comprises a sequence shown in SEQ ID NO: 1 or SEQ ID NO: 10˜SEQ ID NO: 39.

7. The tuber plant or part thereof of claim 1, wherein the part thereof is selected from the group consisting of seed, tuber, fruit, leaf, and flower.

8. The tuber plant or part thereof of claim 1, wherein the plant is potato.

9. A tuber plant or part thereof, comprising (i) a promoter comprising a sequence having at least 90% sequence identity to SEQ ID NO: 9, and (ii) a sequence encoding a polypeptide comprising a sequence having at least 92% sequence identity to SEQ ID NO: 2, wherein the promoter and the sequence are operably linked, the polypeptide is inhibited-expressed, loss-function, or overexpressed, and the development of plant tuber is changed.

10. The tuber plant or part thereof of claim 9, wherein the promoter comprises a sequence shown in SEQ ID NO: 9 or SEQ ID NO: 97˜SEQ ID NO: 131.

11. A method for the production of a tuber plant or part thereof, wherein overexpresses a polypeptide comprising a sequence having at least 92% sequence identity to SEQ ID NO: 2 in a recipient plant to obtain a target plant; compared with a recipient plant, the target plant has the characteristics of earlier formation time of tubers, increased number of tubers, increased size of tubers, and/or increased yield of tubers.

12. The method of claim 11, wherein the formation time of tubers of a target plant is at least 30% earlier than a recipient plant.

13. The method of claim 11, wherein the number, the size, or the yield of tubers for a target plant is at least 30% greater than a recipient plant.

14. The method of claim 11, wherein the plant is potato.

15. The method of claim 11, wherein the overexpression comprises one or more techniques selected from the group consisting of: promoter editing technology, codon optimization, utilization of a strong promoter, insertion of an intron, utilization of a viral vector, and fusion protein technology.

16. A method for the production of a tuber plant or part thereof, wherein loss-function or inhibits the expression of a polypeptide comprising a sequence having at least 92% sequence identity to SEQ ID NO: 2 in a recipient plant, to obtain a target plant; compared with a recipient plant, the target plant does not form a tuber, or has delayed formation time of tubers, reduced number of tubers, reduced size of tubers, and/or reduced yield of tubers.

17. The method of claim 16, wherein the formation time of tubers of a target plant is at least 30% later than a recipient plant.

18. The method of claim 16, wherein the number, the size, or the yield of tubers for a target plant is at least 30% lower than a recipient plant.

19. The method of claim 16, wherein the plant is potato.

20. The method of claim 16, wherein the loss-function or inhibition comprises one or more techniques selected from the group consisting of: CRISPR/Cas9 gene editing technology, TALEN technology, T-DNA insertion, EMS mutagenesis, and ZFN technology.


SEQUENCE LISTING (133 sequences)

SEQ ID NO: 1 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagacctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtagttttgtggttaatggaaattggaatccatttacaatcttcacttctcatgaacaatatgctggaatttccaatgagcatcaattagttacagacttgcaattttatggaaagctgtgggaaagctag

SEQ ID NO: 2 - protein, 364 aa, unidentified
  source 1..364: note = Protein encoded by IT1 gene
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETTAKDEKEKKKKPVNTARRPAFEPLTKESRNQARARARERTKTKKMSQVGKSKSPAHDLNPSGSRRPANRTCEEPGTHEQHTFHHVDDSSFVVNGNWNPFTIFTSHEQYAGISNEHQLVTDLQFYGKLWES

SEQ ID NO: 3 - DNA, 1826 bp, unidentified
  source 1..1826: mol_type = other DNA; note = genomic DNA of potato
catgcctgtagcttgatgcttagacgggtgcacacgcactctctcactcacacagctagaatatatatatatatatatatatatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctaccaactttaattttcttcttattgcagctagcttgcttaattagcagcaaaactcaaaagaggttttagctgtgtttatactgtctttctcaagatctagacccaccacttagaccatctcaagctacagctactgcgcaaatgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagacctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtagttttgtggttaatggaaattggaatccatttacaatcttcacttctcatgaacaatatgctggaatttccaatgaggtgagggcttcaataattaattaaatccagtagatttctatttatatatatatatatatattcttatcagcttctaaaaaaaatccttatttctctgcagcatcaattagttacagacttgcaattttatggaaagctgtgggaaagctagggcaaggaaattcgaagcggacaaagttccttcttcattttgtgcttactcggccgagcagaagaacgtttccggcctgtatctgttggtaaatgtaacatacatttctactttttaagtgtgtttttcttttgtgaaagaacttcatgtgcagatagcctatgttttttttttgaataaaactatgatggggttcattaagcaagcgacatattcggg

SEQ ID NO: 4 - DNA, 20 bp, unidentified
  source 1..20: mol_type = other DNA; note = target site 1
gaattagtattattagtgga

SEQ ID NO: 5 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 6 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 7 - DNA, 20 bp, synthetic construct
  source 1..20: mol_type = other DNA
agctacagctactgcgcaaa

SEQ ID NO: 8 - DNA, 20 bp, synthetic construct
  source 1..20: mol_type = other DNA
agtagtgggggtattgcagc

SEQ ID NO: 9 - DNA, 3000 bp, unidentified
  source 1..3000: mol_type = other DNA; note = the promoter regions of IT1 nucleotide sequences
cacaaattttttaggttgccgatccagagaatttaaatattaatgttgtgaattctaatactccaaatattaatgttgtgaattctaatactccacctgttttaaaaaatggtctagtttgacttgaaacggaatttaagaaaagaaaaaagactttctaatcttgtgatgccaaattaaagttatgtcaaatataccaaaatgtcctttaatcttgtgctcttaaacatgtcacgtgaaaaattgaagttaaagtgttgccaaaaaaagaaaggggtcattattttttaaacaaactaaaaaaaaaaaactaagtcattccttttgaaacggagaaagtatactatatttatatatttggttctcgcaaacctgcaaataagggcggatgcacaatagaagttacgagtttgattaaatctactaattttatttaaactttgtatatatgtgtagagaagtcataaagttcaaatactgaatttactaattttatcaaagttgtgatctactgctaattaaagttgggtgggcaggtggggatgccccgtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggcgcggagctagatatgcaaagggtttatataaatttatgtgatacattttttttagttaatctaaaatataataaaagcttttttttttaacaagatcgatttgaaatcacacaaatatttgtgaatttagatcataaatttcaaaaatcttttaataaaaggctagcttctgttcttggccttttagaggttaggtacatgctccatcccctcctttcttgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacattcctttacttgacttactaaatttaaatatatcttcgatctgccacataacataacaaatggtttcaaacggaacatatgactcttaataaaaggtggagaagggttgaaaactttactaaatttaacaagatcttaggtgtgtatttgactcatgttgcaagattgaagtgtatttaagttcagttaatcaagtaaagagatatttttaaggttgtcaatatataattcggagataaaactagccttttgtttcatttgttgagtttaaaattaagttgatatgaaacaacaaataaattgtgatcccaagttttatatgagcaaaataaaatcaagcattgtatatctctcttttatttttctccctagtatccgcattggagcccgactatattcgaatcgcactataggcttcatttttttcatactcgagtcttaaatctgaaacctctgggtaagggtagaatatcttatatctctttttttttttttttttggattttccattcaacgtttggtacccacattgaagttcagttaattcaaatttgcaccgcataggctaagcggttcctaccaatgatcttttcatacccagggactcaaacacaagaagtttaattaagaaataaatagtctcatccgctataccacattgtgatatatctcattaagataccacattatatctatttatgggggtaggggggatgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctctcactcacacagctagaatatatatatatatatatatatatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctaccaactttaattttcttcttattgcagctagcttgcttaattagcagcaaaactcaaaagaggttttagctgtgtttatactgtctttctcaagatctagacccaccacttagaccatctcaagctacagctactgcgcaa

SEQ ID NO: 10 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcctcacgtcttagctgatagctgcactgagactgttactaagccttcaaattgcaatcacgtactagaaggaatggaagaaggccgaggcggaaacaaaggagatgatgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgttgaatggttgcttactcaatcggataccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacacctcgtagagctgcgtttgaacctcttacaaaggaatcaaggaatcaagcaagagccagggctagagagagaacaaaaacaaagaaaatgagccaagttggaaaatccaaatccccagttcatgatttgaacccttcaggatctcggaggccggctaatagaacttgtgaagaacctggaacacatgaacaacacaccttccatcatgttgatgacagtaattttgtggttaatggaaattggaatccatatacaatcttcacctctcatgaacaatatgctggaatttccaatgagcatcaattagttacagacttgcaattttatggaaagctgtgggaaagctag

SEQ ID NO: 11 - DNA, 1092 bp, unidentified
  source 1..1092: mol_type = other DNA; note = IT1 gene CDS
atgtatcctccaagcaacaataactgcaactacagcccaattttgtcttctttcatatgccaaaatattccatcttctccttgtatgcaatatgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaataagagatcttccaataaggatcgacacagtaagatcaacacggctcgtggtccaagagatcgaaggatgcgactttcacttgatgctgctcgcaagtttttccgtttgcaggacttattgggatttgataaggccagcaaaactgtagaatggttgcttactcaatcggataccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgagcatcaattagttacagacttgcaattttatggaaagctgtgggaaagctag

SEQ ID NO: 12 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactattgctaaagatgagaaggagaaaaagaagaagccggttaacacagctcgtagacctgcgtttgaacctcttacaaaggaatcaaggaatcaagcaagagccagggctagagagagaacaaaatcaaagaaaatgagccaagttggaaaatccaaatccccagctcatgatttgaatccttcaggatctcggaggccggctaatagaacttgtgaagaacctggaacacatgaacaacacaccttccatcatgttgatgacagtagttttgtggttaatggaaattggaatccatttacaatcttcacctctcatgaacaatatgctggaatttccaatgagcatcaattagttacagacttgcaattttatggaaagctgtgggaaagctag

SEQ ID NO: 13 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagacctgcgtttgaacctcttacaaaggaatcaaggaatcaagcaagagccagggctagagagagaacaaaatcaaagaaaatgagccaagttggaaaatccaaatccccagctcatgatttgaacccttcaggatctcggaggccggctaatagaacttgtgaagaacctggaacacatgaacaacacaccttccatcatgttgatgacagtagttttgtggttaatggaaattggaatccatttacaatcttcacctctcatgaacaatatgctggaatttccaatgagcatcaattagttacagacttgcaattttatggaaagctgtgggaaagctag

SEQ ID NO: 14 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttgttgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagacctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtagttttgtggttaatggaaattggaatccatttacaatcttcacttctcatgaacaatatgctggaatttccaatgagcatcaattagttacagacttgcaattttatggaaagctgtgggaaagctag

SEQ ID NO: 15 - DNA, 1095 bp, unidentified
  source 1..1095: mol_type = other DNA; note = IT1 gene CDS
atgtatcctccaagcaacaataactgcaactacagcccaattttgtcttctttaatatgccaaaatattccatcttctccttgtatgcaatatgaacacgaactatactttcaaaacttcaatcatgacgaccaatattattttcaacaacagcaacaagttcccttaatagatgacttgagtcctcacatcttagctgacagctgcaccgagactgttactaagccttcaaattgcaatcacgtacaagaaggaatggaagaaggccgaggcggaaacaaaggaggtgatgttatgagtagcagaattagtattattagtggacggatctcaaaaaataataagagatcttccaataaggatcgacacagcaagatcaacaccgctcgtggtccaagagatcgaaggatgagactttcacttgatgctgctcgcaagtttttccgtttgcaggacttgttgggattcgataaggccagcaaaactgtagaatggttgcttactcaatcggattccgcaattgaagagctcgtcgccgttaaaggcaatgatgctcagattcctcagcaaactagctgcaatactcccactactactactggaattggtgcaatttgtgcatctaattctatttctgagtcatgtgaagttatatcaggaactgatgaaacttcctctaatgacaaaaacaaggtaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagccaaattggaaaatccaaagccccagctcatgatttgaacccttcaggaggatcggctaataaaacttgtgaagaacctggaactcatgaacaacacaccttccatcatgagaagaacagtgtcgatgaaagtaattttgtggttaatggatattggaatccatttacaatcttcagctatcatgagcaatatgctggaatttccaatgagcatcaattagttacagacttgcaattttgtggaaagctgtgggaaagctag

SEQ ID NO: 16 - DNA, 1092 bp, unidentified
  source 1..1092: mol_type = other DNA; note = IT1 gene CDS
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttctcttgatagatgacttgagtcctcacatcttagctgacagctgcacagagactgttactaagccttcaaattgcaatcacgtactcgaaggaatggaagaaggccgaggcggaaacaaaggagatgatgttatgagtagcagaattagtattattagtggacggatctcaaaaaataataagagatcttccaataaggatcgacacagcaagatcaacaccgctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcaccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagccaaattggaaaatccaatgatttgaacccttcaggatctcggaggccggctaatagaacttgtgaagaagaacctggaacacatgaacaacacaccttccatcatgagaagaacagtgttaatgacagtaattttgtggttaatggaaattggaatccatttacaatcttcagctctcatgaacaatatgctggaatttccaatgagcatcaattagttacagacttgcaattttatggaaagctgtgggaaagctag

SEQ ID NO: 17 - DNA, 1101 bp, unidentified
  source 1..1101: mol_type = other DNA; note = IT1 gene CDS
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttcttttgatagatgacttgagtcctcacatcttagctgacagctgcactgagactgttactaagccttcaaattgcaatcacgtactagaaggaatggaagaaggccaaggcggaaacaaaggagatgatgttatgagtagcagaattagtattattagtggacggatctcaaaaaataataagagatcttccaataaggatcgacacagcaagatcaacaccgctcgtggtccaagagatcgaaggatgagactttcacttgatgctgctcgcaagtttttccgtttgcaggacttattgggattcgataaggccagcaaaactgtagaatggttgcttactcaatcggattccgcaattgaagagctcgtcgccgtgaaaggcaatgatgctcaggtttctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagccaaattggaaaatcccaagccccagctcatgatttgaacccttcaggatctcggaggccggctaatagaacttgtgaagaacctggaacacatgaacaacacaccttccatcatgagaagaacagtgttgatgacagtaattttttggttaatggaaattggaatccatttacaatcttcagctctcatgaacaatatgctggaatttccaatgagcatcaattagttacagatttgcaattttatggaaagctgtgggaaagctag

SEQ ID NO: 18 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccatcttctccttgtatgcaatatgaacacgaactatactttcaaaacttcaatcatgatgaccaatattattttcaactacagcaacaagttcccttgatagatgacttgagtcctcacgtcttagctgacagctgcactgaaactgttactaagccttcaaattgcaatcacgtactagaaggaatggaggaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgcgactttcacttgatgctgctcgcaagtttttccgtttgcaggacttattgggatttgataaggccagcaaaactgtagaatggttgcttactcaatcggataccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgaaaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgagcatcaattagttacagacttgcaattttatggaaagctgtgggaaagctag

SEQ ID NO: 19 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattatttccaactacagcaacaagttcccttgatagatgacttgagtcctcacgtcttagctgatagctgcactgagactgttactaagccttcaaattgcaatcacgtactagaaggaatggaagaaggccgaggcggaaacaaaggagatgatgttgttatgagtagcagaattagtattattagtggacggatctcaaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgagcatcaattagttacagacttgcaattttatggaaagctgtgggaaagctag

SEQ ID NO: 20 - DNA, 1101 bp, unidentified
  source 1..1101: mol_type = other DNA; note = IT1 gene CDS
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttctcttgatagatgacttgagtcctcgcatcttagctgacagctgcactgagactgttactaagcgttcaaattgcaatcacgtactagaaggaatggaagaaggccaaggcggaaacaaaggagatgatgttatgagtagcagaattagtatttttagtggacggatctcaaaaaataataagagatcttccaataaggatcgacacagcaagatcaacaccgctcgtggtccaagagatcgaaggatgagactttcacttgatgctgctcgcaagtttttccgtttgcaagacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctacgtttgaacctcttacaaaggaatcaaggaatcaagcaagagccagggctagagagagaacaaaaacaaagaaaatgagccaaattggaaaatccaaagccccagctcatgatttgaacccttcaggatctcggaggccggctaatagaacttgtgaagaacctggaacaaatgaacaacacaccttccatcatgacaagaacagtgttgatgacagtaattttttggttaatggaaattggaatccatttacaatcttcagctctcatgaacaatatgctggaatttccaatgagcatcaattagttacagacttgcaattttatggaaagctgtgggaaagctag

SEQ ID NO: 21 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccgtcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcgtgtgaagttatatcaggaactgatgaaacttcctctaatgacaaaaacaaggaaactactgctaaagatgagaaggagaaaaagaagaagccggttaacacagctcgtagagctgcgtttgaacctcttacgaaggaatcaaggaatcaagcaagagccagggctagagagagaacaaaaacaaagaaaatgagccaagttggaaaatccaaatccccagctcatgatttgaacccttcaggatctcggaggccggctaatagaatttgtgaagaacctggaacacatgaacaacacaccttccatcatgttgatgacagtaattttgtggttaatggaaattggaatccatttacaatcttcacctctcatgaacaatatgctggaatttccaatgagcatcaattagttacagacttgcaattttatggaaagctgtgggaaagctag

SEQ ID NO: 22 - DNA, 1092 bp, unidentified
  source 1..1092: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacctgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaataagagatcttccaataaggatcgacacagcaagatcaacacggctcgtggtccaagagatcgaaggatgcgactttcacttgatgctgctcgcaagtttttccgtttgcaggacttactgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaacagaacttgtgaagaacctggaacacatgaacaacacaccttccatcatgttgatgacagtaattttgtggttaatggaaattggaatccatttacaatcttcagctctcatgaacaatatgctggaatttccaatgagcatcaattagttacagacttgcaattttatggaaagctgtgggaaagctag

SEQ ID NO: 23 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccgtcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgctgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgagcatcaattagttacagacttgcaattttatggaaagctgtgggaaagctag

SEQ ID NO: 24 - DNA, 1095 bp, unidentified
  source 1..1095: mol_type = other DNA; note = IT1 gene CDS
atgtatcctccaagcaacaataactgcaactacagcccaattttgtcttctttaatatgccaaaatattccatcttctccttgtatgcaatatgaacacgaactatactttcaaaacttcaatcatgatgaccaatattattttcaacaacagcaacaagctcccttgatagatgacttgagtcctcacatcttagctgacagctgcaccgagactgttactaagccttcaaattgcaatcacgtactagaaggaatggaagaaggccgaggcggaaacaaaggagatgatgttatgagtagcagaattagtattattagtggacggatctcaaaaaataataagagatcttccaataaggatcgacacagcaagatcaacaccgctcgtggtccaagagatcgaaggatgagactttcacttgatgctgctcgcaagtttttccgtttgcaggacttgttgggattcgataaggccagcaaaactgtagaatggttgcttactcaatcggattccgcaattgaagagctcgtcgccgttaaaggcaatgatgctcagattcctcagcaaactagctgcaatacccccactgctactactggaataggtgcaatttgtgcatctaattctatttctgagtcatgtgaagttatatcaggaactgatgaaacttcctctaatgacaaaaacaaggtaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagccaaattggaaaatccaaagccccagctcatgatttgaacccttcaggaggatcggctaataaaacttgtgaagaacctggaactcatgaacaacacaccttccatcatgagaagaacagtgtcgatgaaagtaattttgtggttaatggatattggaatccatttacaatcttcagttatcatgagcaatatgctggagtttccaatgagcatcaattagttacagacttgcaattttgtggaaagctgtgggaaagctag

SEQ ID NO: 25 - DNA, 1101 bp, unidentified
  source 1..1101: mol_type = other DNA; note = IT1 gene CDS
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttcccttgatagatgacttgagtcctcacatcttagctgacagctgcactgagactgttactaagccttcaaattgcaatcacgtactagaaggaatggaagaaggccaaggcggaaacaaaggagatgatgttatgagtagcagaattagtattattagtggacggatctcaaaaaataataagagatcttccaataaggatcgacacagcaagatcaacactgctcgtggtccaagagatcgaaggatgagactttcacttgatgctgctcgcaaatttttccgtttgcaggacttattgggattcgataaggccagcaaaactgtagaatggttgcttactcaatcggattccgcaattgaagagctcgtcgccgttaaagggaatgatgctcaggttcctcagcaaactagctgcaatacccccactactactactggaattggtgcaatttgtgcatctaattctatttctgagtcatgtgaagttatatcaggaactgatgaaacttcatctaatgacaaaaacaaggaaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagtcaaattggaaaatccaaatccccagctcatgatttgaacccttcaggatctcggaggccggctaatagaacttgtgaagaacctggaacacatgaacaacacaccttccatcatgagaagaacagtgttgatgacagtaattttttggttaatggaaattggaatccatttacaatcttcagctctcatgaacaatatgctggaatttccaatgagcatcaattagttacagacttgcaattttatggaaagctgtgggaaagctag

SEQ ID NO: 26 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaaccgcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtagttttgtggttaatggaaattggaatccatttacaatcttcacctctcatgaacaatatgctggaatttccaatgagcatcaattagttacagacttgcaattttatggaaagctgtgggaaagctag

SEQ ID NO: 27 - DNA, 1092 bp, unidentified
  source 1..1092: mol_type = other DNA; note = IT1 gene CDS
atgtatcctccaagcaacaataactgcaactacagcccaattttgtcttctttaatatgccaaaatattccatcttctccttgtatgcaatatgaacatgaactatactttcaaaactttaatcatgatgaccattattattttcaacaacagcaacaagttcccttgatagatgacttgagtcctcacatcttagctgacagctgcaccgagactgttactaagccttcaaactgcaatcacgtactagaaggaatggaagaaggccgaggcggaaacaaaggagatgatgttatgagtagcagaattagtattattagtggacggatctcaaaaaataacaagagatcttccaataaggatcgacacagcaagatcaacaccgctcgtggtccaagagatcgaaggatgagactttcactcgatgccgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctgcgtttgaacctcttacaaaggaatcaaggaatcaagcaagagccagggctagagagagaacaaaaacaaagaagatgagccaaattggaaaatccaaagccccagctcatgatttgaacctttcaggatcggctagtaaaacttgtgaagaacctggaactcatgaacaacacaacttccatcatgagaagatcagtgtcgatgaaagtaattttgttgttaatggaaattggaatccatttacaatcttcagctctcatgaacaatatgctggaatgtccaatgagcatcaattagttacagacttgcagttttatggaaagctgtgggaaagctag

SEQ ID NO: 28 - DNA, 1101 bp, unidentified
  source 1..1101: mol_type = other DNA; note = IT1 gene CDS
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttctcttgatagatgacttgagtcctcacatcttagctgacagctgcactgagactgttactaagccttcaaattgcaatcacgtactagaaggaatggaagaaggccaaggcggaaacaaaggagatgatgttatgagtagcagaattagtattattagtggacggatctcaaaaaataataagagatcttccaataaggatcgacacagcaagatcaacaccgctcgtggtccaagagatcgaaggatgagactttcacttgacgctgctcgcaagtttttccgtttgcaggacttattgggattcgataaggccagcaaaactgtagaatggttgcttactcaatcggattccgcaattgaagagctcgtcgccgttaaaggcaatgatgctcaggttcctcagcaaactagctgtaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagccaaattggaaaatccaaagccccagctcatgatttgaacccttcaggatctcggaggccggctaatagaacttgtgaagaacctggaacacatgaacaacacaccttccatcatgagaagaacagtgttgatgacagtaattttttggttaatggaaattggaatccatttacaatcttcagctctcatgaacaatatgctggaatttccaatgagcatcaattagttacagacttgcaattttatggaaagctgtgggaaagctag

SEQ ID NO: 29 - DNA, 1089 bp, unidentified
  source 1..1089: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacctgagtcctcacgtcttagctgacagctgcactgagactgttactaagccttcaaattgcaatcacgtactagaaggaatggaagaaggccaaggcggaaacaaaggagatgatgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcagctctcatgaacaatatgctggaatttccagtgagcatcaattagttacagacttgcaattttatggaaagctgtgggaaagctag

SEQ ID NO: 30 - DNA, 1203 bp, unidentified
  source 1..1203: mol_type = other DNA; note = IT1 gene CDS
atgtatcctccaagcaacaataactgcaactacagcccaattttgtcttctttaatattccaaaatattccatcttctccttgtatgcaatatgaacacgaactatactttcaaaactttaatcatgatgaccaatattattttcaactacagcaacaagttcccttgatagatgacttgaatcctcacatcttagctgatagctgcacagagactgttactaagccttcaaattgcaatcacgtactagaaggaatggaagaaggccgaggcggaaacaaaggagaggatgctatgagtagcagaattagtattattagtggacggatctcgaaaaacaataagagatcttccaataaggatcgacacagcaagatcaacaccgctcgtggtccaagagatcgaaggatgagactttcacttgatgccgctcgcaagtttttccgtttgcaggacttattgggattcgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaagccggttaacacaggtcgtagagctgcgtttgaacctcttacaaaggaatcaaggaatcaagcaagagccagggctagagagagaacaaaaacaaagaaaatgagccaaattggaaaatccaaatcctcagctcatgatttgaacccttcaggatctcggaggccggctactaatagaacttgtgaagaacctggaacacatgaacaacacaccttccatcatgagaagaacagtgttgatgacagtaattttgtggttaatggaaattggaatccatttacaatcttcagctctcatgaacaatatgctggaatttccaatgagacttgcaattttatggaaagctgtgggaaagctagggcaaggaaattcgaagcggacaaagttccttcttcattttgtgcttactcggccgagcagaagaacgtttccggcctgtatctgttggtaaatgtaacatacattcgtactttt

SEQ ID NO: 31 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaataagagatcttccaataaggatcgacacagcaagatcaacacggctcgtggtccaagagatcgaaggatgcgactttcacttgatgctgctcgcaagtttttccgtttgcaggacttgttgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcagctctcatgaacaatatgctggaatttccaatgagcagcatcaattagttacagacttgcaattttatggaaagctgtgggaaagctag

SEQ ID NO: 32 - DNA, 1092 bp, unidentified
  source 1..1092: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaacaagagatcttccaataaggatcgacacagcaagatcaacacggctcgtggtccaagagatcgaaggatgcgactttcacttgatgctgctcgcaagtttttccgtttgcaggacttattgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagtcaagttggaaaatccaaatccccagctcatgaattgaacccttcaggatctcggaggccggctaatagaacttgtgaagaacctggaacacatgaacaacacaccttccatcatgttgatgacagtaattttgtggttaatggaaattggaatccatttacaatcttcagctctcatgaacaatatgctggaatttccagtgagcatcaattagtgacagacttgcaattttatggaaagctgtgggaaagctag

SEQ ID NO: 33 - DNA, 1080 bp, unidentified
  source 1..1080: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcacgatgaccaatattattttcaactacagcaacaagttcccctgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcaaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaagccggctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagatcttgtgaagaacctgcaacacatgaacaacacaccttccatcatgttgatgacagtaattttgtggttaatggaaattggaatccatttacaatcttcagctctcatgaacaatatgctggaatttccaatgagcatcaattagttacagacttgcaattttatggaaagctgtgggaaagctag

SEQ ID NO: 34 - DNA, 1092 bp, unidentified
  source 1..1092: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggatactgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgagcatcaattagttacagacttgcaattttatggaaagctgtgggaaagctag

SEQ ID NO: 35 - DNA, 1095 bp, unidentified
  source 1..1095: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgatagctgcactgagactgttactaaaccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgagcatcaattagttacagacttgcaattttatggaaagctgtgggaaagctag

SEQ ID NO: 36 - DNA, 1092 bp, unidentified
  source 1..1092: mol_type = other DNA; note = IT1 gene CDS
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaaattcccttgatagatgacttgagtcctcacatcttagctgagagctgcaccgagactgttactaagccttcaaattgcaatcacgtactagaaggaatggaagaaggccgaggcggaaacaaaggagatgatgttatgagtagcagaattagtattattagtggacggatctcaaaaaataataagagatcttccaataaggatcgacacagcaagatcaacaccgctcgtggtccaagagatcgaaggatgagactttcactcgatgctgctcgcaagtttttccgtttgcaggacttgttgggattcgataaggccagcaaaactgtagaatggttgcttactcaatcggattccgcaattgaagagctcgtcgccgttaaaggcaatgatgctcagattcctcagcaaactagctgcaatacccccactactactactggaattggtgcaatttgtgcatctaattctatttctgagtcatgtgaagttatatcaggaactgatgaaacttcctctaatgacaaaaacaaggtaactgctaaagatgagaaggagaaaaagaagaaggcggttaacacagctcgtagagctgcgtttgaacctcttacaaaggaatcaaggaatcaagcaagagctagggctagagagagaacaaaaacaaagaaaatgagccaaattggaaaatccaaagccccagctcatgatttgaacccttcaggatcggctaataaaacttgtgaagaacctggaactcatgaacaacacaccttccatcatgagaagaacagcgtcgatgaaagtaattttgtggttaatggaaattggaatccatttacaatcttcagctatcatgaacaatatgctggaatttccaacgagcatcaattagttacagacttgcaattttgtggaaagctgtgggaaagctag

SEQ ID NO: 37 - DNA, 1101 bp, unidentified
  source 1..1101: mol_type = other DNA; note = IT1 gene CDS
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttctcttgatagatgacttgagtcctcacatcttagctgacagctgcactgagactgttactaagccttcaaattgcaatcacgtactagaaggaatggaagaaggccaaggcggaaacaaaggagatgatgttatgagtagtagaattagtattattagtggacggatctcgaaaaacaataagagatcttccaataaggatcgacacagcaagatcaacaccgctcgtggtccaagagatcgaaggatgagactttcacttgatgctgctcgcaagtttttccgtttgcaggacttattgggattcgataaggccagcaaaactgtagaatggttgcttactcaatcggattccgcaattgaagagctcgtcgccgttaaaggcaatgatgctcaggttcctcagcaaactagctgcaataccaccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctgcgtttgaacctcttacaaaggaatcaaggaatcaagcaagagctagggctagagagagaacaaaaacaaagaaaatgagccaaattggaaaatccaaagccccagctcatgatttgaacccttcaggatctcggaggccagctaatagaacttgtgaagaacccggaacacatgaacaacacaccttccatcatgagaagaacagtgttgatgacagtaattttgtggttaatggaaattggaatccatttacaatcttcagctctcatgaacaatatgctggaatttccaatgagcatcaattagttacagacttgcaattttatggaaagctgtgggaaagctag

SEQ ID NO: 38 - DNA, 1089 bp, unidentified
  source 1..1089: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacccgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcagctctcatgaacaatatgctggaatttccaatgagcatcaattagttacagacctgcaattttatggaaagctgtgggaaagctag

SEQ ID NO: 39 - DNA, 1089 bp, unidentified
  source 1..1089: mol_type = other DNA; note = IT1 gene CDS
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcaaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggcttcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagttcatgatttgaacccttcaggatctcggaggccggctaatagaacttgtgaagaacctggaacacatgaacaacacaccttccatcatgttgatgacactaattttgtggttaatggaaattggaatccatttacaatcttcagctctcatgaacaatatgctggaatttccaatgagcatcaattagttacagacttgcaattttatggaaagctgtgggaaagctag

SEQ ID NO: 40 - protein, 364 aa, unidentified
  source 1..364: note = Protein encoded by IT1 gen
MYPPSNNNCNYSPILSSFICQNIPSSPCMQYEHELYFQNFNHDDQYYFQLQQQVPLIDDLSPPHVLADSCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDTAIEELVAVKGNDAQVPQQTSCNTPTTTTGIGAICASNSISESCEVISGTDETSSNDKNKETTAKDEKEKKKKPVNTPRRAAFEPLTKESRNQARARARERTKTKKMSQVGKSKSPVHDLNPSGSRRPANRTCEEPGTHEQHTFHHVDDSNFVVNGNWNPYTIFTSHEQYAGISNEHQLVTDLQFYGKLWES

SEQ ID NO: 41 - protein, 363 aa, unidentified
  source 1..363: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDTAIEELVAVKGNDAQVPQQTSCNTPTTTTGIGAICASNSISESCEVISGTDETSSNDKNKETTAKDEKEKKKKPVNTARRAAFEPLTKESRNQARARARERTKTKKMSQVGKSKSPAHDLNPSGSRRPANRTCEEPGTHEQHTFHHVDDSNFVVNGNWNPFTIFTSHEQYAGISNEHQLVTDLQFYGKLWES

SEQ ID NO: 42 - protein, 364 aa, unidentified
  source 1..364: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETIAKDEKEKKKKPVNTARRPAFEPLTKESRNQARARARERTKSKKMSQVGKSKSPAHDLNPSGSRRPANRTCEEPGTHEQHTFHHVDDSSFVVNGNWNPFTIFTSHEQYAGISNEHQLVTDLQFYGKLWES

SEQ ID NO: 43 - protein, 364 aa, unidentified
  source 1..364: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETTAKDEKEKKKKPVNTARRPAFEPLTKESRNQARARARERTKSKKMSQVGKSKSPAHDLNPSGSRRPANRTCEEPGTHEQHTFHHVDDSSFVVNGNWNPFTIFTSHEQYAGISNEHQLVTDLQFYGKLWES

SEQ ID NO: 44 - protein, 364 aa, unidentified
  source 1..364: note = Protein encoded by IT1 gen
MYPPSNNNCNYSPILSSLICQNIPSSPCMQYEHELYFQNFNHDDQYYFQQQQQVPLIDDLSPHILADSCTETVTKPSNCNHVQEGMEEGRGGNKGGDVMSSRISIISGRISKNNKRSSNKDRHSKINTARGPRDRRMRLSLDAARKFFRLQDLLGFDKASKTVEWLLTQSDSAIEELVAVKGNDAQIPQQTSCNTPTTTTGIGAICASNSISESCEVISGTDETSSNDKNKVTAKDEKEKKKKAVNTARRAAFEPLTKESRNQARARARERTKTKKMSQIGKSKAPAHDLNPSGGSANKTCEEPGTHEQHTFHHEKNSVDESNFVVNGYWNPFTIFSYHEQYAGISNEHQLVTDLQFCGKLWES

SEQ ID NO: 45 - protein, 363 aa, unidentified
  source 1..363: note = Protein encoded by IT1 gen
MYPPSNNNCNYSPILSSLICQNIPSSPCMQYEHELYFQNFNHDDQYYFQQQQQVLLIDDLSPHILADSCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDSAIEELVTVKGNDAQVPQQTSCNTPTTTTGIGAICASNSISESCEVISGTDETSSNDKNKETAKDEKEKKKKAVNTARRAAFEPLTKESRNQARARARERTKTKKMSQIGKSNDLNPSGSRRPANRTCEEEPGTHEQHTFHHEKNSVNDSNFVVNGNWNPFTIFSSHEQYAGISNEHQLVTDLQFYGKLWES

SEQ ID NO: 46 - protein, 366 aa, unidentified
  source 1..366: note = Protein encoded by IT1 gen
MYPPSNNNCNYSPILSSLICQNIPSSPCMQYEHELYFQNFNHDDQYYFQQQQQVLLIDDLSPHILADSCTETVTKPSNCNHVLEGMEEGQGGNKGDDVMSSRISIISGRISKNNKRSSNKDRHSKINTARGPRDRRMRLSLDAARKFFRLQDLLGFDKASKTVEWLLTQSDSAIEELVAVKGNDAQVSQQTSCNTPTTTTGIGAICASNSISESCEVISGTDETSSNDKNKETAKDEKEKKKKAVNTARRAAFEPLTKESRNQARARARERTKTKKMSQIGKSQAPAHDLNPSGSRRPANRTCEEPGTHEQHTFHHEKNSVDDSNFLVNGNWNPFTIFSSHEQYAGISNEHQLVTDLQFYGKLWES

SEQ ID NO: 47 - protein, 364 aa, unidentified
  source 1..364: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVVMSSRISIISGRISKNNKRSSNKDRHSKINTARGPRDRRMRLSLDAARKFFRLQDLLGFDKASKTVEWLLTQSDTAIEELVAVKGNDAQVPQQTSCNTPTTTTGIGAICASNSISESCEVISGTDETSSNDKNKETTAKDEKEKKKKPVNTARRAAFEPLTKESRNQARARARERTKTKKMSQVGKSKSPAHDLNPSGSRRPANRTCEEPGTHEQHTFHHVDDSNFVVNGNWNPFTIFTSHEQYAGISNEHQLVTDLQFYGKLWES

SEQ ID NO: 48 - protein, 364 aa, unidentified
  source 1..364: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETTAKDEKEKKKKPVNTARRAAFEPLTKESRNQARARARERTKTKKMSQVGKSKSPAHDLNPSGSRRPANRTCEEPGTHEQHTFHHVDDSNFVVNGNWNPFTIFTSHEQYAGISNEHQLVTDLQFYGKLWES

SEQ ID NO: 49 - protein, 366 aa, unidentified
  source 1..366: note = Protein encoded by IT1 gen
MYPPSNNNCNYSPILSSLICQNIPSSPCMQYEHELYFQNFNHDDQYYFQQQQQVLLIDDLSPRILADSCTETVTKRSNCNHVLEGMEEGQGGNKGDDVMSSRISIFSGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETAKDEKEKKKKAVNTARRATFEPLTKESRNQARARARERTKTKKMSQIGKSKAPAHDLNPSGSRRPANRTCEEPGTNEQHTFHHDKNSVDDSNFLVNGNWNPFTIFSSHEQYAGISNEHQLVTDLQFYGKLWES

SEQ ID NO: 50 - protein, 364 aa, unidentified
  source 1..364: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETTAKDEKEKKKKPVNTARRAAFEPLTKESRNQARARARERTKTKKMSQVGKSKSPAHDLNPSGSRRPANRICEEPGTHEQHTFHHVDDSNFVVNGNWNPFTIFTSHEQYAGISNEHQLVTDLQFYGKLWES

SEQ ID NO: 51 - protein, 363 aa, unidentified
  source 1..363: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETTAKDEKEKKKKPVNTARRAAFEPLTKESRNQARARARERTKTKKMSQVGKSKSPAHDLNPSGSRRPANRTCEEPGTHEQHTFHHVDDSNFVVNGNWNPFTIFSSHEQYAGISNEHQLVTDLQFYGKLWES

SEQ ID NO: 52 - protein, 364 aa, unidentified
  source 1..364: note = Protein encoded by IT1 gen
MYPPSNNNCNYSPILSSLICQNIPSSPCMQYEHELYFQNFNHDDQYYFQQQQQAPLIDDLSPHILADSCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDSAIEELVAVKGNDAQIPQQTSCNTPTATTGIGAICASNSISESCEVISGTDETSSNDKNKVTAKDEKEKKKKAVNTARRAAFEPLTKESRNQARARARERTKTKKMSQIGKSKAPAHDLNPSGGSANKTCEEPGTHEQHTFHHEKNSVDESNFVVNGYWNPFTIFSYHEQYAGVSNEHQLVTDLQFCGKLWES

SEQ ID NO: 53 - protein, 366 aa, unidentified
  source 1..366: note = Protein encoded by IT1 gen
MYPPSNNNCNYSPILSSLICQNIPSSPCMQYEHELYFQNFNHDDQYYFQQQQQVPLIDDLSPHILADSCTETVTKPSNCNHVLEGMEEGQGGNKGDDVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETAKDEKEKKKKAVNTARRAAFEPLTKESRNQARARARERTKTKKMSQIGKSKSPAHDLNPSGSRRPANRTCEEPGTHEQHTFHHEKNSVDDSNFLVNGNWNPFTIFSSHEQYAGISNEHQLVTDLQFYGKLWES

SEQ ID NO: 54 - protein, 364 aa, unidentified
  source 1..364: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETTAKDEKEKKKKPVNTARRAAFEPLTKESRNQARARARERTKTKKMSQVGKSKSPAHDLNPSGSRRPANRTCEEPGTHEQHTFHHVDDSSFVVNGNWNPFTIFTSHEQYAGISNEHQLVTDLQFYGKLWES

SEQ ID NO: 55 - protein, 363 aa, unidentified
  source 1..363: note = Protein encoded by IT1 gen
MYPPSNNNCNYSPILSSLICQNIPSSPCMQYEHELYFQNFNHDDHYYFQQQQQVPLIDDLSPHILADSCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETAKDEKEKKKKAVNTARRAAFEPLTKESRNQARARARERTKTKKMSQIGKSKAPAHDLNLSGSASKTCEEPGTHEQHNFHHEKISVDESNFVVNGNWNPFTIFSSHEQYAGMSNEHQLVTDLQFYGKLWES

SEQ ID NO: 56 - protein, 366 aa, unidentified
  source 1..366: note = Protein encoded by IT1 gen
MYPPSNNNCNYSPILSSLICQNIPSSPCMQYEHELYFQNFNHDDQYYFQQQQQVLLIDDLSPHILADSCTETVTKPSNCNHVLEGMEEGQGGNKGDDVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETAKDEKEKKKKAVNTARRAAFEPLTKESRNQARARARERTKTKKMSQIGKSKAPAHDLNPSGSRRPANRTCEEPGTHEQHTFHHEKNSVDDSNFLVNGNWNPFTIFSSHEQYAGISNEHQLVTDLQFYGKLWES

SEQ ID NO: 57 - protein, 362 aa, unidentified
  source 1..362: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGQGGNKGDDVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETAKDEKEKKKKPVNTARRAAFEPLTKESRNQARARARERTKTKKMSQVGKSKSPAHDLNPSGSRRPANRTCEEPGTHEQHTFHHVDDSNFVVNGNWNPFTIFSSHEQYAGISSEHQLVTDLQFYGKLWES

SEQ ID NO: 58 - protein, 401 aa, unidentified
  source 1..401: note = Protein encoded by IT1 gen
MYPPSNNNCNYSPILSSLIFQNIPSSPCMQYEHELYFQNFNHDDQYYFQLQQQVPLIDDLNPHILADSCTETVTKPSNCNHVLEGMEEGRGGNKGEDAMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETAKDEKEKKKKPVNTGRRAAFEPLTKESRNQARARARERTKTKKMSQIGKSKSSAHDLNPSGSRRPATNRTCEEPGTHEQHTFHHEKNSVDDSNFVVNGNWNPFTIFSSHEQYAGISNETCNFMESCGKARARKFEADKVPSSFCAYSAEQKNVSGLYLLVNVTYIRTF

SEQ ID NO: 59 - protein, 364 aa, unidentified
  source 1..364: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETTAKDEKEKKKKPVNTARRAAFEPLTKESRNQARARARERTKTKKMSQVGKSKSPAHDLNPSGSRRPANRTCEEPGTHEQHTFHHVDDSNFVVNGNWNPFTIFSSHEQYAGISNEQHQLVTDLQFYGKLWES

SEQ ID NO: 60 - protein, 363 aa, unidentified
  source 1..363: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETTAKDEKEKKKKPVNTARRAAFEPLTKESRNQARARARERTKTKKMSQVGKSKSPAHELNPSGSRRPANRTCEEPGTHEQHTFHHVDDSNFVVNGNWNPFTIFSSHEQYAGISSEHQLVTDLQFYGKLWES

SEQ ID NO: 61 - protein, 359 aa, unidentified
  source 1..359: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETAKDEKEKKKKPARRAAFEPLTKESRNQARARARERTKTKKMSQVGKSKSPAHDLNPSGSRRPANRSCEEPATHEQHTFHHVDDSNFVVNGNWNPFTIFSSHEQYAGISNEHQLVTDLQFYGKLWES

SEQ ID NO: 62 - protein, 363 aa, unidentified
  source 1..363: note = Protein encoded by IT1 gen
MYPPSNNNCNYSPILSSLICQNIPSSPCMQYEHELYFQNFNHDDQYYFQQQQQIPLIDDLSPHILAESCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDSAIEELVAVKGNDAQIPQQTSCNTPTTTTGIGAICASNSISESCEVISGTDETSSNDKNKVTAKDEKEKKKKAVNTARRAAFEPLTKESRNQARARARERTKTKKMSQIGKSKAPAHDLNPSGSANKTCEEPGTHEQHTFHHEKNSVDESNFVVNGNWNPFTIFSYHEQYAGISNEHQLVTDLQFCGKLWES

SEQ ID NO: 63 - protein, 366 aa, unidentified
  source 1..366: note = Protein encoded by IT1 gen
MYPPSNNNCNYSPILSSLICQNIPSSPCMQYEHELYFQNFNHDDQYYFQQQQQVLLIDDLSPHILADSCTETVTKPSNCNHVLEGMEEGQGGNKGDDVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTTTTTTGIGAICASNSISESCEVISGTDETSSNDKNKETAKDEKEKKKKAVNTARRAAFEPLTKESRNQARARARERTKTKKMSQIGKSKAPAHDLNPSGSRRPANRTCEEPGTHEQHTFHHEKNSVDDSNFVVNGNWNPFTIFSSHEQYAGISNEHQLVTDLQFYGKLWES

SEQ ID NO: 64 - protein, 362 aa, unidentified
  source 1..362: note = Protein encoded by IT1 gen
MYPPSNNNCNYSPILSSFICQNIPSSPCMQYEHELYFQNFNHDDQYYFQLQQQVPLIDDPSPHVLADSCTETVTKPSNCNHVLEGMEEGRGGNKGDDVMSSRISIISGRISKNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETTAKDEKEKKKKPVNTARRAAFEPLTKESRNQARARARERTKTKKMSQVGKSKSPAHDLNPSGSRRPANRTCEEPGTHEQHTFHHVDDSNFVVNGNWNPFTIFSSHEQYAGISNEHQLVTDLQFYGKLWES

SEQ ID NO: 65 - protein, 362 aa, unidentified
  source 1..362: note = Protein encoded by IT1 gen
MYPPSNNNCNYSPILSSFICQNIPSSPCMQYEHELYFQNFNHDDQYYFQLQQQVPLIDDLSPHVLADSCTETVTKPSNCNHVLEGMEEGRGGNKGDDVMSSRISIISGRISKNNKRSSNKDRHSKINTARGPRDRRMRLSLDAARKFFRLQDLLGFDKASKTVEWLLTQSDSAIEELVAVKGNDAQVPQQTSCNTPTTTTGIGAICASNSISESCEVISGTDETSSNDKNKETAKDEKEKKKKPVNTARRAAFEPLTKESRNQARARARERTKTKKMSQVGKSKSPVHDLNPSGSRRPANRTCEEPGTHEQHTFHHVDDTNFVVNGNWNPFTIFSSHEQYAGISNEHQLVTDLQFYGKLWES

SEQ ID NO: 66 - DNA, 1176 bp, unidentified
  source 1..1176: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcctcacgtcttagctgatagctgcactgagactgttactaagccttcaaattgcaatcacgtactagaaggaatggaagaaggccgaggcggaaacaaaggagatgatgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgttgaatggttgcttactcaatcggataccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacacctcgtagagctgcgtttgaacctcttacaaaggaatcaaggaatcaagcaagagccagggctagagagagaacaaaaacaaagaaaatgagccaagttggaaaatccaaatccccagttcatgatttgaacccttcaggatctcggaggccggctaatagaacttgtgaagaacctggaacacatgaacaacacaccttccatcatgttgatgacagtaattttgtggttaatggaaattggaatccatatacaatcttcacctctcatgaacaatatgctggaatttccaatgaggtgagggcttcaataattaaatccagtaaatttctatttatatatattcttatcagcttctgaattcttatttctctgcagcatcaattagttacagacttgcaattttatggaaagctgtgggaaagctag

SEQ ID NO: 67 - DNA, 1183 bp, unidentified
  source 1..1183: mol_type = other DNA; note = genomic DNA of potato
atgtatcctccaagcaacaataactgcaactacagcccaattttgtcttctttcatatgccaaaatattccatcttctccttgtatgcaatatgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaataagagatcttccaataaggatcgacacagtaagatcaacacggctcgtggtccaagagatcgaaggatgcgactttcacttgatgctgctcgcaagtttttccgtttgcaggacttattgggatttgataaggccagcaaaactgtagaatggttgcttactcaatcggataccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgaggtgaggccttcaataattaaatccagtagatttctatttatatatatatatattcttatcagtttctaaaaaaatccttatttctctgcagcatcaattagttacagacttgcaattttatggaaagctgtgggaaagctag

SEQ ID NO: 68 - DNA, 1199 bp, unidentified
  source 1..1199: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagacctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtagttttgtggttaatggaaattggaatccatttacaatcttcacttctcatgaacaatatgctggaatttccaatgaggtgagggcttcaataattaattaaatccagtagatttctatttatatatatatatatatatatattcttatcagcttctaaaaaaaatccttatttctctgcagcatcaattagttacagacttgcaattttatggaaagctgtgggaaagctag

SEQ ID NO: 69 - DNA, 1190 bp, unidentified
  source 1..1190: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactattgctaaagatgagaaggagaaaaagaagaagccggttaacacagctcgtagacctgcgtttgaacctcttacaaaggaatcaaggaatcaagcaagagccagggctagagagagaacaaaatcaaagaaaatgagccaagttggaaaatccaaatccccagctcatgatttgaatccttcaggatctcggaggccggctaatagaacttgtgaagaacctggaacacatgaacaacacaccttccatcatgttgatgacagtagttttgtggttaatggaaattggaatccatttacaatcttcacctctcatgaacaatatgctggaatttccaatgaggtgagggcttcaataattaattaaatccagtagatttctattatatatatatatattcttatcagcttctaaaaaaattccttatttctctgcagcatcaattagttacagacttgcaattttatggaaagctgtgggaaagctag

SEQ ID NO: 70 - DNA, 1183 bp, unidentified
  source 1..1183: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagacctgcgtttgaacctcttacaaaggaatcaaggaatcaagcaagagccagggctagagagagaacaaaatcaaagaaaatgagccaagttggaaaatccaaatccccagctcatgatttgaacccttcaggatctcggaggccggctaatagaacttgtgaagaacctggaacacatgaacaacacaccttccatcatgttgatgacagtagttttgtggttaatggaaattggaatccatttacaatcttcacctctcatgaacaatatgctggaatttccaatgaggtgagggcttcaataattaattaaatccagtagatatatatatatatattcttatcagcttctaaaaaaattccttatttctctgcagcatcaattagttacagacttgcaattttatggaaagctgtgggaaagctag

SEQ ID NO: 71 - DNA, 1199 bp, unidentified
  source 1..1199: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttgttgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagacctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtagttttgtggttaatggaaattggaatccatttacaatcttcacttctcatgaacaatatgctggaatttccaatgaggtgagggcttcaataattaattaaatccagtagatttctatttatatatatatatatatatatattcttatcagcttctaaaaaaaatccttatttctctgcagcatcaattagttacagacttgcaattttatggaaagctgtgggaaagctag

SEQ ID NO: 72 - DNA, 1217 bp, unidentified
  source 1..1217: mol_type = other DNA; note = genomic DNA of potato
atgtatcctccaagcaacaataactgcaactacagcccaattttgtcttctttaatatgccaaaatattccatcttctccttgtatgcaatatgaacacgaactatactttcaaaacttcaatcatgacgaccaatattattttcaacaacagcaacaagttcccttaatagatgacttgagtcctcacatcttagctgacagctgcaccgagactgttactaagccttcaaattgcaatcacgtacaagaaggaatggaagaaggccgaggcggaaacaaaggaggtgatgttatgagtagcagaattagtattattagtggacggatctcaaaaaataataagagatcttccaataaggatcgacacagcaagatcaacaccgctcgtggtccaagagatcgaaggatgagactttcacttgatgctgctcgcaagtttttccgtttgcaggacttgttgggattcgataaggccagcaaaactgtagaatggttgcttactcaatcggattccgcaattgaagagctcgtcgccgttaaaggcaatgatgctcagattcctcagcaaactagctgcaatactcccactactactactggaattggtgcaatttgtgcatctaattctatttctgagtcatgtgaagttatatcaggaactgatgaaacttcctctaatgacaaaaacaaggtaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagccaaattggaaaatccaaagccccagctcatgatttgaacccttcaggaggatcggctaataaaacttgtgaagaacctggaactcatgaacaacacaccttccatcatgagaagaacagtgtcgatgaaagtaattttgtggttaatggatattggaatccatttacaatcttcagctatcatgagcaatatgctggaatttccaatgaggtaagggtttcagaatttgtttattagggcttcaataattaaatccagattatataaatagatgatttccatttatatattcttatcagtttctgagtttttccttatttctctgcacgcagcatcaattagttacagacttgcaattttgtggaaagctgtgggaaagctag

SEQ ID NO: 73 - DNA, 1215 bp, unidentified
  source 1..1215: mol_type = other DNA; note = genomic DNA of potato
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttctcttgatagatgacttgagtcctcacatcttagctgacagctgcacagagactgttactaagccttcaaattgcaatcacgtactcgaaggaatggaagaaggccgaggcggaaacaaaggagatgatgttatgagtagcagaattagtattattagtggacggatctcaaaaaataataagagatcttccaataaggatcgacacagcaagatcaacaccgctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcaccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagccaaattggaaaatccaatgatttgaacccttcaggatctcggaggccggctaatagaacttgtgaagaagaacctggaacacatgaacaacacaccttccatcatgagaagaacagtgttaatgacagtaattttgtggttaatggaaattggaatccatttacaatcttcagctctcatgaacaatatgctggaatttccaatgaggtgagggtttcagactttgtttattagggcttcaataattaaatccagtagatttctatttatttatatatatatatatcagcttctgattttttttgaaaaaaaattcttatttctctgcagcatcaattagttacagacttgcaattttatggaaagctgtgggaaagctag

SEQ ID NO: 74 - DNA, 1235 bp, unidentified
  source 1..1235: mol_type = other DNA; note = genomic DNA of potato
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttcttttgatagatgacttgagtcctcacatcttagctgacagctgcactgagactgttactaagccttcaaattgcaatcacgtactagaaggaatggaagaaggccaaggcggaaacaaaggagatgatgttatgagtagcagaattagtattattagtggacggatctcaaaaaataataagagatcttccaataaggatcgacacagcaagatcaacaccgctcgtggtccaagagatcgaaggatgagactttcacttgatgctgctcgcaagtttttccgtttgcaggacttattgggattcgataaggccagcaaaactgtagaatggttgcttactcaatcggattccgcaattgaagagctcgtcgccgtgaaaggcaatgatgctcaggtttctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagccaaattggaaaatcccaagccccagctcatgatttgaacccttcaggatctcggaggccggctaatagaacttgtgaagaacctggaacacatgaacaacacaccttccatcatgagaagaacagtgttgatgacagtaattttttggttaatggaaattggaatccatttacaatcttcagctctcatgaacaatatgctggaatttccaatgaggtgagggtttcagactttgtttattagggcttcaataattaaatccagtagatttctattttttatatatatatatatatatattcttatcagcttctgattttttttgaaaaaaaattcttatttctctgcagcatcaattagttacagatttgcaattttatggaaagctgtgggaaagctag

SEQ ID NO: 75 - DNA, 1198 bp, unidentified
  source 1..1198: mol_type = other DNA; note = genomic DNA of potato
atgtatcctccaagcaacaataactgcaactacagcccaattttgtcttctttcatatgccaaaatattccatcttctccttgtatgcaatatgaacacgaactatactttcaaaacttcaatcatgatgaccaatattattttcaactacagcaacaagttcccttgatagatgacttgagtcctcacgtcttagctgacagctgcactgaaactgttactaagccttcaaattgcaatcacgtactagaaggaatggaggaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgcgactttcacttgatgctgctcgcaagtttttccgtttgcaggacttattgggatttgataaggccagcaaaactgtagaatggttgcttactcaatcggataccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgaaaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgaggtgagggcttcaataattaaatccagtagatttctatttatttatatatatattcttatcagcttctgattttttttgaaaaaaaattcttatttatctgcagcatcaattagttacagacttgcaattttatggaaagctgtgggaaagctag

SEQ ID NO: 76 - DNA, 1194 bp, unidentified
  source 1..1194: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattatttccaactacagcaacaagttcccttgatagatgacttgagtcctcacgtcttagctgatagctgcactgagactgttactaagccttcaaattgcaatcacgtactagaaggaatggaagaaggccgaggcggaaacaaaggagatgatgttgttatgagtagcagaattagtattattagtggacggatctcaaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgaggtgagggtttcaataattaaatccagtagatttctatttatatgtatattcttatcagcttctgattttttttgaaaaaaaattcttatttctctgcagcatcaattagttacagacttgcaattttatggaaagctgtgggaaagctag

SEQ ID NO: 77 - DNA, 1210 bp, unidentified
  source 1..1210: mol_type = other DNA; note = genomic DNA of potato
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttctcttgatagatgacttgagtcctcgcatcttagctgacagctgcactgagactgttactaagcgttcaaattgcaatcacgtactagaaggaatggaagaaggccaaggcggaaacaaaggagatgatgttatgagtagcagaattagtatttttagtggacggatctcaaaaaataataagagatcttccaataaggatcgacacagcaagatcaacaccgctcgtggtccaagagatcgaaggatgagactttcacttgatgctgctcgcaagtttttccgtttgcaagacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctacgtttgaacctcttacaaaggaatcaaggaatcaagcaagagccagggctagagagagaacaaaaacaaagaaaatgagccaaattggaaaatccaaagccccagctcatgatttgaacccttcaggatctcggaggccggctaatagaacttgtgaagaacctggaacaaatgaacaacacaccttccatcatgacaagaacagtgttgatgacagtaattttttggttaatggaaattggaatccatttacaatcttcagctctcatgaacaatatgctggaatttccaatgaggtgagggtttcagactttgtttattagggcttcaataattaaatccagtagatttctatatatatcagcttctgattttttttgaaaaaaaattcttatttctctgcagcatcaattagttacagacttgcaattttatggaaagctgtgggaaagctag

SEQ ID NO: 78 - DNA, 1196 bp, unidentified
  source 1..1196: mol_type = other DNA; note = genomic DNA of potato
atgtatcctccaagcaacaataactgcaactacagcccaattttgtcttctttcatatgccaaaatattccgtcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcgtgtgaagttatatcaggaactgatgaaacttcctctaatgacaaaaacaaggaaactactgctaaagatgagaaggagaaaaagaagaagccggttaacacagctcgtagagctgcgtttgaacctcttacgaaggaatcaaggaatcaagcaagagccagggctagagagagaacaaaaacaaagaaaatgagccaagttggaaaatccaaatccccagctcatgatttgaacccttcaggatctcggaggccggctaatagaatttgtgaagaacctggaacacatgaacaacacaccttccatcatgttgatgacagtaattttgtggttaatggaaattggaatccatttacaatcttcacctctcatgaacaatatgctggaatttccaatgaggtgagggcttcaataattaattaaatccagtagatttctatttttatatatatatatatatatatatacttatcagcttctgaattcttatttctctgcagcatcaattagttacagacttgcaattttatggaaagctgtgggaaagctag

SEQ ID NO: 79 - DNA, 1179 bp, unidentified
  source 1..1179: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacctgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaataagagatcttccaataaggatcgacacagcaagatcaacacggctcgtggtccaagagatcgaaggatgcgactttcacttgatgctgctcgcaagtttttccgtttgcaggacttactgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaacagaacttgtgaagaacctggaacacatgaacaacacaccttccatcatgttgatgacagtaattttgtggttaatggaaattggaatccatttacaatcttcagctctcatgaacaatatgctggaatttccaatgaggtgagggcttcaattattaaatccagtagattcctatttatatatatattcttatcagcttctaaaaaaattcttatttctctgcagcatcaattagttacagacttgcaattttatggaaagctgtgggaaagctag

SEQ ID NO: 80 - DNA, 1197 bp, unidentified
  source 1..1197: mol_type = other DNA; note = genomic DNA of potato
atgtatcctccaagcaacaataactgcaactacagcccaattttgtcttctttcatatgccaaaatattccgtcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgctgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgaggtgagggcttcaataattaattaaatccagtagatttctatttttatatatatatatatatattcttatcagcttctaaaaaaaatccttatttctctgcagcatcaattagttacagacttgcaattttatggaaagctgtgggaaagctag

SEQ ID NO: 81 - DNA, 1217 bp, unidentified
  source 1..1217: mol_type = other DNA; note = genomic DNA of potato
atgtatcctccaagcaacaataactgcaactacagcccaattttgtcttctttaatatgccaaaatattccatcttctccttgtatgcaatatgaacacgaactatactttcaaaacttcaatcatgatgaccaatattattttcaacaacagcaacaagctcccttgatagatgacttgagtcctcacatcttagctgacagctgcaccgagactgttactaagccttcaaattgcaatcacgtactagaaggaatggaagaaggccgaggcggaaacaaaggagatgatgttatgagtagcagaattagtattattagtggacggatctcaaaaaataataagagatcttccaataaggatcgacacagcaagatcaacaccgctcgtggtccaagagatcgaaggatgagactttcacttgatgctgctcgcaagtttttccgtttgcaggacttgttgggattcgataaggccagcaaaactgtagaatggttgcttactcaatcggattccgcaattgaagagctcgtcgccgttaaaggcaatgatgctcagattcctcagcaaactagctgcaatacccccactgctactactggaataggtgcaatttgtgcatctaattctatttctgagtcatgtgaagttatatcaggaactgatgaaacttcctctaatgacaaaaacaaggtaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagccaaattggaaaatccaaagccccagctcatgatttgaacccttcaggaggatcggctaataaaacttgtgaagaacctggaactcatgaacaacacaccttccatcatgagaagaacagtgtcgatgaaagtaattttgtggttaatggatattggaatccatttacaatcttcagttatcatgagcaatatgctggagtttccaatgaggtaagggtttcagaatttgtttattagggcttcaataattaaatccagattatataaatagatgatttccatttatatattcttatcagtttctgagtttttccttatttctctgcacgcagcatcaattagttacagacttgcaattttgtggaaagctgtgggaaagctag

SEQ ID NO: 82 - DNA, 1224 bp, unidentified
  source 1..1224: mol_type = other DNA; note = genomic DNA of potato
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttcccttgatagatgacttgagtcctcacatcttagctgacagctgcactgagactgttactaagccttcaaattgcaatcacgtactagaaggaatggaagaaggccaaggcggaaacaaaggagatgatgttatgagtagcagaattagtattattagtggacggatctcaaaaaataataagagatcttccaataaggatcgacacagcaagatcaacactgctcgtggtccaagagatcgaaggatgagactttcacttgatgctgctcgcaaatttttccgtttgcaggacttattgggattcgataaggccagcaaaactgtagaatggttgcttactcaatcggattccgcaattgaagagctcgtcgccgttaaagggaatgatgctcaggttcctcagcaaactagctgcaatacccccactactactactggaattggtgcaatttgtgcatctaattctatttctgagtcatgtgaagttatatcaggaactgatgaaacttcatctaatgacaaaaacaaggaaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagtcaaattggaaaatccaaatccccagctcatgatttgaacccttcaggatctcggaggccggctaatagaacttgtgaagaacctggaacacatgaacaacacaccttccatcatgagaagaacagtgttgatgacagtaattttttggttaatggaaattggaatccatttacaatcttcagctctcatgaacaatatgctggaatttccaatgaggtgagggtttcagactttgtttattagggcttcaataattaaatccagtagatttctatttatatatatatattcttatcagcttctgattttttttgaaaaaaaattcttatttttctgcagcatcaattagttacagacttgcaattttatggaaagctgtgggaaagctag

SEQ ID NO: 83 - DNA, 1189 bp, unidentified
  source 1..1189: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaaccgcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtagttttgtggttaatggaaattggaatccatttacaatcttcacctctcatgaacaatatgctggaatttccaatgaggtgagggcttcaataattaattaaatccagtagatttctatttatatatatatattcttatcagcttctaaaaaaaatccttatttctctgcagcatcaattagttacagacttgcaattttatggaaagctgtgggaaagctag

SEQ ID NO: 84 - DNA, 1214 bp, unidentified
  source 1..1214: mol_type = other DNA; note = genomic DNA of potato
atgtatcctccaagcaacaataactgcaactacagcccaattttgtcttctttaatatgccaaaatattccatcttctccttgtatgcaatatgaacatgaactatactttcaaaactttaatcatgatgaccattattattttcaacaacagcaacaagttcccttgatagatgacttgagtcctcacatcttagctgacagctgcaccgagactgttactaagccttcaaactgcaatcacgtactagaaggaatggaagaaggccgaggcggaaacaaaggagatgatgttatgagtagcagaattagtattattagtggacggatctcaaaaaataacaagagatcttccaataaggatcgacacagcaagatcaacaccgctcgtggtccaagagatcgaaggatgagactttcactcgatgccgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctgcgtttgaacctcttacaaaggaatcaaggaatcaagcaagagccagggctagagagagaacaaaaacaaagaagatgagccaaattggaaaatccaaagccccagctcatgatttgaacctttcaggatcggctagtaaaacttgtgaagaacctggaactcatgaacaacacaacttccatcatgagaagatcagtgtcgatgaaagtaattttgttgttaatggaaattggaatccatttacaatcttcagctctcatgaacaatatgctggaatgtccaatgaggtgagggtttcagactttgtttattagggcttcaataattaaatccagtagatttctatttatatataatattcttatcagcttctgattttttttgaaaaaaaattcttatttctctgcagcatcaattagttacagacttgcagttttatggaaagctgtgggaaagctag

SEQ ID NO: 85 - DNA, 1230 bp, unidentified
  source 1..1230: mol_type = other DNA; note = genomic DNA of potato
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttctcttgatagatgacttgagtcctcacatcttagctgacagctgcactgagactgttactaagccttcaaattgcaatcacgtactagaaggaatggaagaaggccaaggcggaaacaaaggagatgatgttatgagtagcagaattagtattattagtggacggatctcaaaaaataataagagatcttccaataaggatcgacacagcaagatcaacaccgctcgtggtccaagagatcgaaggatgagactttcacttgacgctgctcgcaagtttttccgtttgcaggacttattgggattcgataaggccagcaaaactgtagaatggttgcttactcaatcggattccgcaattgaagagctcgtcgccgttaaaggcaatgatgctcaggttcctcagcaaactagctgtaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctgcgtttgaacctcttacaaaggaatcaaggaatcaagcaagagccagggctagagagagaacaaaaacaaagaaaatgagccaaattggaaaatccaaagccccagctcatgatttgaacccttcaggatctcggaggccggctaatagaacttgtgaagaacctggaacacatgaacaacacaccttccatcatgagaagaacagtgttgatgacagtaattttttggttaatggaaattggaatccatttacaatcttcagctctcatgaacaatatgctggaatttccaatgaggtgagggtttcagactttgtttattagggcttcaataattaaatccagtagatttctatttatatatatatatatatattcttatcagcttctgattttttttgaaatttttttcttatttctctgcagcatcaattagttacagacttgcaattttatggaaagctgtgggaaagctag

SEQ ID NO: 86 - DNA, 1190 bp, unidentified
  source 1..1190: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacctgagtcctcacgtcttagctgacagctgcactgagactgttactaagccttcaaattgcaatcacgtactagaaggaatggaagaaggccaaggcggaaacaaaggagatgatgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcagctctcatgaacaatatgctggaatttccagtgaggtgagggcttcaataattaaatccagtagatttctatttatatatatatattcttatcagcttctgattttttttgaaaaaaatttcttatttctctgcagcatcaattagttacagacttgcaattttatggaaagctgtgggaaagctag

SEQ ID NO: 87 - DNA, 1316 bp, unidentified
  source 1..1316: mol_type = other DNA; note = genomic DNA of potato
atgtatcctccaagcaacaataactgcaactacagcccaattttgtcttctttaatattccaaaatattccatcttctccttgtatgcaatatgaacacgaactatactttcaaaactttaatcatgatgaccaatattattttcaactacagcaacaagttcccttgatagatgacttgaatcctcacatcttagctgatagctgcacagagactgttactaagccttcaaattgcaatcacgtactagaaggaatggaagaaggccgaggcggaaacaaaggagaggatgctatgagtagcagaattagtattattagtggacggatctcgaaaaacaataagagatcttccaataaggatcgacacagcaagatcaacaccgctcgtggtccaagagatcgaaggatgagactttcacttgatgccgctcgcaagtttttccgtttgcaggacttattgggattcgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaagccggttaacacaggtcgtagagctgcgtttgaacctcttacaaaggaatcaaggaatcaagcaagagccagggctagagagagaacaaaaacaaagaaaatgagccaaattggaaaatccaaatcctcagctcatgatttgaacccttcaggatctcggaggccggctactaatagaacttgtgaagaacctggaacacatgaacaacacaccttccatcatgagaagaacagtgttgatgacagtaattttgtggttaatggaaattggaatccatttacaatcttcagctctcatgaacaatatgctggaatttccaatgaggtgagggcttcaataattaaatccagtagatttctatttatatatattcttatcagcttctgattttttttgaaaaaaaattcttatttctctgcagcatcaattagttacagacttgcaattttatggaaagctgtgggaaagctagggcaaggaaattcgaagcggacaaagttccttcttcattttgtgcttactcggccgagcagaagaacgtttccggcctgtatctgttggtaaatgtaacatacattcgtactttt

SEQ ID NO: 88 - DNA, 1179 bp, unidentified
  source 1..1179: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaataagagatcttccaataaggatcgacacagcaagatcaacacggctcgtggtccaagagatcgaaggatgcgactttcacttgatgctgctcgcaagtttttccgtttgcaggacttgttgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcagctctcatgaacaatatgctggaatttccaatgaggtgagggcttcaataattaaatccagtagatttctatttatatatatattcttatcagcttctaaaaaaattcttatttctcagcagcatcaattagttacagacttgcaattttatggaaagctgtgggaaagctag

SEQ ID NO: 89 - DNA, 1205 bp, unidentified
  source 1..1205: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaacaagagatcttccaataaggatcgacacagcaagatcaacacggctcgtggtccaagagatcgaaggatgcgactttcacttgatgctgctcgcaagtttttccgtttgcaggacttattgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagtcaagttggaaaatccaaatccccagctcatgaattgaacccttcaggatctcggaggccggctaatagaacttgtgaagaacctggaacacatgaacaacacaccttccatcatgttgatgacagtaattttgtggttaatggaaattggaatccatttacaatcttcagctctcatgaacaatatgctggaatttccagtgaggtgagggcatcaataattaaatccagtagatttctacttatatatatatatatatatattcttatcagcttctgattttttttgaaaaaaaaaaaaatcttatttctctgcagcatcaattagtgacagacttgcaattttatggaaagctgtgggaaagctag

SEQ ID NO: 90 - DNA, 1175 bp, unidentified
  source 1..1175: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcacgatgaccaatattattttcaactacagcaacaagttcccctgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcaaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaagccggctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagatcttgtgaagaacctgcaacacatgaacaacacaccttccatcatgttgatgacagtaattttgtggttaatggaaattggaatccatttacaatcttcagctctcatgaacaatatgctggaatttccaatgaggtgagggcttcaataattaaatccagtagatttttctatatatatatatatattcttatgattttttttgaaaaaaaattcttatttctctgcagcatcaattagttacagacttgcaattttatggaaagctgtgggaaagctag

SEQ ID NO: 91 - DNA, 1183 bp, unidentified
  source 1..1183: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggatactgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgaggtgagggcttcaataattataattaaatccagtagatttctatttttatatatatattctcatcagcttctgaattcttatttctctgcagcatcaattagttacagacttgcaattttatggaaagctgtgggaaagctag

SEQ ID NO: 92 - DNA, 1180 bp, unidentified
  source 1..1180: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgatagctgcactgagactgttactaaaccttcaaattgcaatcacgtactagaaggaatggaagaaggccgaggcggaaacaaaggagatgatgttgttatgagtagcagaattagtattattagtggacggatctcgaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcggattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcacctctcatgaacaatatgctggaatttccaatgaggtgagggcttcaataattaaatccagtagatttatatatatatatattcttatcagcttctaaaaaaatccttatttctctgcagcatcaattagttacagacttgcaattttatggaaagctgtgggaaagctag

SEQ ID NO: 93 - DNA, 1189 bp, unidentified
  source 1..1189: mol_type = other DNA; note = genomic DNA of potato
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaaattcccttgatagatgacttgagtcctcacatcttagctgagagctgcaccgagactgttactaagccttcaaattgcaatcacgtactagaaggaatggaagaaggccgaggcggaaacaaaggagatgatgttatgagtagcagaattagtattattagtggacggatctcaaaaaataataagagatcttccaataaggatcgacacagcaagatcaacaccgctcgtggtccaagagatcgaaggatgagactttcactcgatgctgctcgcaagtttttccgtttgcaggacttgttgggattcgataaggccagcaaaactgtagaatggttgcttactcaatcggattccgcaattgaagagctcgtcgccgttaaaggcaatgatgctcagattcctcagcaaactagctgcaatacccccactactactactggaattggtgcaatttgtgcatctaattctatttctgagtcatgtgaagttatatcaggaactgatgaaacttcctctaatgacaaaaacaaggtaactgctaaagatgagaaggagaaaaagaagaaggcggttaacacagctcgtagagctgcgtttgaacctcttacaaaggaatcaaggaatcaagcaagagctagggctagagagagaacaaaaacaaagaaaatgagccaaattggaaaatccaaagccccagctcatgatttgaacccttcaggatcggctaataaaacttgtgaagaacctggaactcatgaacaacacaccttccatcatgagaagaacagcgtcgatgaaagtaattttgtggttaatggaaattggaatccatttacaatcttcagctatcatgaacaatatgctggaatttccaacgaggtaagggtttcagaatttgtttattagggcttcaataattaaatccagattataattcttatcagtttctgagtttttcctttttctctgcacgcagcatcaattagttacagacttgcaattttgtggaaagctgtgggaaagctag

SEQ ID NO: 94 - DNA, 1244 bp, unidentified
  source 1..1244: mol_type = other DNA; note = genomic DNA of potato
atgtatcctccaagcaacaataactgcaactacagcccaattttgtcttctttaatatgccaaaatattccatcttctccttgtatgcaatatgaacacgaactatactttcaaaactttaatcatgatgaccaatattattttcaacaacagcaacaagttctcttgatagatgacttgagtcctcacatcttagctgacagctgcactgagactgttactaagccttcaaattgcaatcacgtactagaaggaatggaagaaggccaaggcggaaacaaaggagatgatgttatgagtagtagaattagtattattagtggacggatctcgaaaaacaataagagatcttccaataaggatcgacacagcaagatcaacaccgctcgtggtccaagagatcgaaggatgagactttcacttgatgctgctcgcaagtttttccgtttgcaggacttattgggattcgataaggccagcaaaactgtagaatggttgcttactcaatcggattccgcaattgaagagctcgtcgccgttaaaggcaatgatgctcaggttcctcagcaaactagctgcaataccaccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaaggcggttaacacagctcgtagagctgcgtttgaacctcttacaaaggaatcaaggaatcaagcaagagctagggctagagagagaacaaaaacaaagaaaatgagccaaattggaaaatccaaagccccagctcatgatttgaacccttcaggatctcggaggccagctaatagaacttgtgaagaacccggaacacatgaacaacacaccttccatcatgagaagaacagtgttgatgacagtaattttgtggttaatggaaattggaatccatttacaatcttcagctctcatgaacaatatgctggaatttccaatgaggtgagggtttcagactttgtttattagggcttcaataactaaatccagtagatttctatttatatatatatatatatatatatatatatatattcttatcagcttctgattatttttttaaaaaaattcttatttctctgcagcatcaattagttacagacttgcaattttatggaaagctgtgggaaagctag

SEQ ID NO: 95 - DNA, 1195 bp, unidentified
  source 1..1195: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacccgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcgaaaaacaagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggttgcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagctcatgatttgaacccttcaggatctcggaggccggctaatagaacttgtgaagaacctggaacacatgaacaacacaccttccatcatgttgatgacagtaattttgtggttaatggaaattggaatccatttacaatcttcagctctcatgaacaatatgctggaatttccaatgaggtgagggctacaataattaatccagtagatttctatttatatatatatatatatattcttatcagcttctgattttttttgaaaaaaaattcttatttctctgcagcatcaattagttacagacctgcaattttatggaaagctgtgggaaagctag

SEQ ID NO: 96 - DNA, 1187 bp, unidentified
  source 1..1187: mol_type = other DNA; note = genomic DNA of potato
atgtatcctccaagcaacaataactgcaactacagcccaattttgtcttctttcatatgccaaaatattccatcttctccttgtatgcaatacgaacacgaactatactttcaaaacttcaatcatgatgaccaatattattttcaactacagcaacaagttcccttgatagatgacttgagtcctcacgtcttagctgacagctgcactgagactgttactaagccttcaaattgcaatcacgtactagaaggaatggaagaaggccgaggcggaaacaaaggagatgatgttatgagtagcagaattagtattattagtggacggatctcaaaaaacaataagagatcttccaataaggatcgacacagcaagatcaacacggctcgtggtccaagagatcgaaggatgagactttcacttgatgctgctcgcaagtttttccgtttgcaggacttattgggatttgataaggccagcaaaactgtagaatggcttcttactcaatcagattccgcaattgaagagctcgtcgccgttaaaggcaatgatgctcaggttcctcagcaaactagctgcaatacccccactactactactggaattggtgcaatttgtgcatctaattctatttctgagtcatgtgaagttatatcaggaactgatgaaacttcctctaatgacaaaaacaaggaaactgctaaagatgagaaggagaaaaagaagaagccggttaacacagctcgtagagctgcgtttgaacctcttacaaaggaatcaaggaatcaagcaagagccagggctagagagagaacaaaaacaaagaaaatgagccaagttggaaaatccaaatccccagttcatgatttgaacccttcaggatctcggaggccggctaatagaacttgtgaagaacctggaacacatgaacaacacaccttccatcatgttgatgacactaattttgtggttaatggaaattggaatccatttacaatcttcagctctcatgaacaatatgctggaatttccaatgaggtgatggcttcaataattaaatccagtagatttctatttatatatatatatatatattcttatcagcttctaaaaaaaaaattcttatttctctgcagcatcaattagttacagacttgcaattttatggaaagctgtgggaaagctag

SEQ ID NO: 97 - DNA, 3000 bp, unidentified
  source 1..3000: mol_type = other DNA; note = the promoter regions of IT1 nucleotide sequences
ttaatgttgtgaattctaatactccaaatattaatgttgtgaattctaatactccacctgttttaaaaaatggtctagtttgacttgaaacggaatttaagaaaagaaaaaagactttctaatcttgtgatgccaaattaaagttatgtcaaatataccaaaatgtcctttaatcttgtgctcttaaacatgtcacgtgaaaaattgaagttaaagtgttgccaaaaaaagaaaggggtcattattttttaaacaaactaaaaaaaaaaaactaagtcattccttttgaaacggagaaagtatactatatttatatatttggttctcgcaaacctgcaaataagggcggatgcacaatagaagttacgagtttgattaaatctactaattttatttaaactttgtatatatgtgtagagaagtcataaagttcaaatactgaatttactaattttatcaaagttgtgatctactgctaattaaagttgggtgggcaggtggggatgccccgtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggcgcagagctagatatgcaaagggtttatataaatttatgtgatacattttttttagttaatctaaaaaaagaataaaagtttttttttttaccaagatctatttgaaatcacacaaatatttgtgaatttagatcataaatttcaaaaatcttttaataaaaggttagcttctgttcttggccttttagaggttaggtacatgctccatcccctcatttcttgttgttactatttgccatggccggtaggaatcaaggtcaaaccccaatgcatcacaggttagtatagtccgaccgtgacaattttctatgggggattgtgtagaatcaaatatctattcataaattgagacattgagacgaatattcttctttacattattgagacgaaaaaaatagtatttaattttcttaataaaaatcttagctccccactatttggtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagtctttctttttaagaagaataaaaaacatacctcaatttggtataaaccaatagtttcgtcaatctaaaaaatattcttttacttgactcactaaatttagatatatctttgatctgtcacataacataacataacaaatgatttcaaacttttgtcggaacatatgacttaataaaaggtggagaaaggttgaaaactttactaaatttaacaagatcctaggtgtgtatttgactcatgttgcaagattgaagcgtatttaagtttagttagtcaagtaaagagatatttttaaggttgtcaatatataattcagagataaaactaataattcacactgaatttagggggtgttttcaatactcttcttttgtttcatttcttgagtttaaaattaagttgatatggaacaacaaataaattgtgatcccaagttttatatgagtaaaataaaatcaagcattgtatatctctcttttatttttctccctggtgtccgcattggagcccgactatatttgattcacactataggcttcatttttttcatactcagggcttaaatctgaaatctgtgggtaagggtagaatatcttatatctcttttttttttttttttttagattttccatccaacgtttgttacccacattgaagttcagttaattcaaatttgcaccgcataggctaagcggttcctaccaatgatcttttcatacctagggattcaaacgcaaaacatttatttaagaaataaatagtctcatccgctatatagtgtggtatatctcattaaaaagataccacattatatctatttaaggggggtaggggggatgggtctagctagggttgtggaaatgaggttagagagagggtggtttttcagtgtgtgtgtcaggtgaaatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctcactcacacagctagaatatatatattcatagttagcagaagtacttttcatataccaaaaaccacacaaatacattgtatcaagtgctgtcatactcgagcaaaagaaagaaaagaacaagatatagtagtactactgttttcatcaccaattttggtcaatcatgatgattctgaacaaagatatagtactagctaggtagaaaataaatctagcaactttaattttcttcttattgcagctagcttgcttaattagtagcaaaatcaaaagaggttttagctgtgtttatactgtctttctcaagatctagacccaccacttagaccatctcaagctacagctactgcgcaa

SEQ ID NO: 98 - DNA, 3000 bp, unidentified
  source 1..3000: mol_type = other DNA; note = the promoter regions of IT1 nucleotide sequences
taagctgattcggtgggaaaatcatgttttatatcacaaattttttaggttgccgatccagagaatttaaatattaatgttgtgaattctaatactccacctgttttaaaaaatggtctagtttgacttgaaacggagtttaagaaaagaaaaaagactttctaatcttgtgattccaaattaaagttatgtcaaatgtaccaaaatgtcctttaatcttgtggtcttaaacatgtcacgtgaaaaattgaagttaaagtgttgcaaaaaaaaaaggggtcattcttttttaaacaaactaaaaaagaaaactaagtcattccttttgaaacggaggatgtatactatatttatatatttggttctcgcaaacctgcaaataagggcggatgcacaacagaagttacgagtttgattaaatcttctaattttagtttaaactttgtatatatgcgtagagaagtcataaagttcaaatactgaatttactaattttatcaaagttgtgatctactgctgattaaagttgggtgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggcgcggagctagatatgcaaagggtttatataaatttatgtgatacattttttttagttaatctaaaatataataaaagcttttttttttaacaagatcgatttgaaatcacacaaatatttgtgaatttagatcataaatttcaaaaatcttttaataaaaggctagcttctgttcttggccttttagaggttaggtacatgctccatcccctcctttcttgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacattcctttacttgacttactaaatttaaatatatcttcgatctgccacataacataacaaatggtttcaaacggaacatatgactcttaataaaaggtggagaagggttgaaaactttactaaatttaacaagatcttaggtgtgtatttgactcatgttgcaagattgaagtgtatttaagttcagttaatcaagtaaagagatatttttaaggttgtcaatatataattcggagataaaactagccttttgtttcatttgttgagtttaaaattaagttgatatgaaacaacaaataaattgtgatcccaagttttatatgagcaaaataaaatcaagcattgtatatctctcttttatttttctccctagtatccgcattggagcccgactatattcgaatcgcactataggcttcatttttttcatactcgagtcttaaatctgaaacctctgggtaagggtagaatatcttatatctctttttttttttttttggattttccattcaacgtttggtacccacattgaagttcagttaattcaaatttgcaccgcataggctaagcggttcctaccaatgatcttttcatacccagggactcaaacacaagaagtttaattaagaaataaatagtctcatccgctataccacattgtgatatatctcattaagataccacattatatctatttatgggggtaggggggatgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctctcactcacacagctagaatatatatatatatatatatatatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctaccaactttaattttcttcttattgcagctagcttgcttaattagcagcaaaactcaaaagaggttttagctgtgtttatactgtctttctcaagatctagacccaccacttagaccatctcaagctacagctactgcgcaa

SEQ ID NO: 99 - DNA, 3000 bp, unidentified
  source 1..3000: mol_type = other DNA; note = the promoter regions of IT1 nucleotide sequences
aagctgattcggtgggaaaatcatgttttatatcacaaattttttaggttgccgatccagagaatttaaatattaatgttgtgaattctaatactccacctgttttaaaaaatggtctagtttgacttgaaacggagtttaagaaaagaaaaaagactttctaatcttgtgattccaaattaaagttatgtcaaatgtaccaaaatgtcctttaatcttgtggtcttaaacatgtcacgtgaaaaattgaagttaaagtgttgcaaaaaaaaaaaggggtcattcttttttaaacaaactaaaaaagaaaactaagtcattccttttgaaacggaggatgtatactatatttatatatttggttctcgcaaacctgcaaataagggcggatgcacaacagaagttacgagtttgattaaatcttctaattttagtttaaactttgtatatatgcgtagagaagtcataaagttcaaatactgaatttactaattttatcaaagttgtgatctactgctgattaaagttgggtgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggcgcggagctagatatgcaaagggtttatataaatttatgtgatacattttttttagttaatctaaaatataataaaagcttttttttttaacaagatcgatttgaaatcacacaaatatttgtgaatttagatcataaatttcaaaaatcttttaataaaaggctagcttctgttcttggccttttagaggttaggtacatgctccatcccctcctttcttgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacattcctttacttgacttactaaatttaaatatatcttcgatctgccacataacataacaaatggtttcaaacggaacatatgactcttaataaaaggtggagaagggttgaaaactttactaaatttaacaagatcttaggtgtgtatttgactcatgttgcaagattgaagtgtatttaagttcagttaatcaagtaaagagatatttttaaggttgtcaatatataattcggagataaaactagccttttgtttcatttgttgagtttaaaattaagttgatatgaaacaacaaataaattgtgatcccaagttttatatgagcaaaataaaatcaagcattgtatatctctcttttatttttctccctagtatccgcattggagcccgactatattcgaatcgcactataggcttcatttttttcatactcgagtcttaaatctgaaacctctgggtaagggtagaatatcttatatctctttttttttttttttggattttccattcaacgtttggtacccacattgaagttcagttaattcaaatttgcaccgcataggctaagcggttcctaccaatgatcttttcatacccagggactcaaacacaagaagtttaattaagaaataaatagtctcatccgctataccacattgtgatatatctcattaagataccacattatatctatttatgggggtaggggggatgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctctcactcacacagctagaatatatatatatatatatatatatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctaccaactttaattttcttcttattgcagctagcttgcttaattagcagcaaaactcaaaagaggttttagctgtgtttatactgtctttctcaagatctagacccaccacttagaccatctcaagctacagctactgcgcaa

SEQ ID NO: 100 - DNA, 3000 bp, unidentified
  source 1..3000: mol_type = other DNA; note = the promoter regions of IT1 nucleotide sequences
tgttgtgaattctaatactccacctgttttaaaaaatggtctagtttgattaggaatggagtttaagaaaaaaaaaaaagattttctaatcttggagttcaaaattaaagttatgttaaatgtaccaaaatatcctttaattttgtggtcctaaacatgtcacgtgaaaaattgaagctaaagtgttaccaaaaaaagaaaggggtcatcctttttgaaacaaacaaaaaaaaaactaagtcattattttttaaacgaagaaagtatactatatttatatatttggttctcgcaaacctgcaaataagggcggatgcacaacagaagttacgagtttgattaaatctactaattttagtttaaactttgtatatatgtgtaaaggagtcataaagttcaaatactgaatttactaattttatcaaagttgtgatctactgctaattaaagttggatgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagaccttcatcactccctagtgagggcgaagagctagatatgcaaaggatttatataaatttatgtgatacgttttttttatttaatctaaaaaataataagttttttttttttaccaagatcgatttgaaatcacacaaatatttgtgaatttagatcaaatatttgtgaatttagatcataaatttcaaaagtcttttaataaaaggctagcttctgttcttggccttttagaggttaggtacatgctccatcccctccttacttgttgttactatttgccatggctggtagggatcagggtcaaaccccaatgtatcacaggtcaatatagtccgaccgtgacaattttctagggtgtattgtatagaatcaaatatctattcataaattgagacattgagacgaatattcttctttacattattgagacgaagataatattaaattttcttaataaaaatcttagctcctcactatttggtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctctatttggtataaaccaatagtttcgtcaatcttaaaaacattcttttacttgactcactaaatttaaatatatcttcgatctgccacataacataacaaataatttcaaactcttgtcaaaacatatgactcttaataaaaggtggagaagggttgaaaactttactaaatttaacaagatcttaggtgtgtatttgactcatgttgcaagattgaagtgtatttaagttcagttagtcaagtaaaaagatatttttaaggttgtcaatatataattcggagataaaactaataatgcacactgaatttagagggtgttttcgacacttttcttttgtttcatttgttcagtttaaaattaagttgatatgaaacaacaaataaattgtgatcccaagttttatatgagcaaaataaaatcaagcattgtatatctctcttttatttttttccctggtgtccgcattggagctcgactatattcgaatcatactataagcttcatttttttcatactcaaggcttaaatctgaaacctctgggtaagggtagaaaatcttatatctctttcttttttttggatttttcatctaacgtttggtacccacattgaaattcaattaattcaaatttgcgccgcataggctaagcggttcctacaaatgatctttccatatccagggactcaaacgcaaaatatttatttaagaaataaatagtctcatatgctataccacatcagttgatagtgtgatatatctcattaagataccacattatatctatttaagggggtggggggacgggtctagctagggttgtggaaatgaggttagagagagggtggtttttcagtgtgtgtgtcaggtgaaatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctcactctcactcacacagctagaatatatatattcatatatatatattcatagttagcagaagtacttatcctataccaaaaaccatacaaatacattgtatcaagtgctgtcatactcaagcaaaagaaagaaaagaacaagatatagtagtactactgttttcatcaccattttggtcaatcatgatgattctgaacaaagatatagtactagctaggtagaaaataaatctactaactttaattttcttcttattgcagctagcttgcttaattagcagcaaaactcaaaagaggttttagctgtgtttatactgtctttctcaagatctagacccaccacttagaccatctcaagctacagctactgcgcaa

SEQ ID NO: 101 - DNA, 3000 bp, unidentified
  source 1..3000: mol_type = other DNA; note = the promoter regions of IT1 nucleotide sequences
ttcggagggaaaatcatgttttatatcacaaattttttaggttgccgatccagagaatttaaatattaatgttgtgaattctaatactccacctgttttaaaaaatgatctagtttgacttgaaacggagtttaagaaaagaaaaaagactttctaatcttgtgattccaaattaaagttatatcaaatgtaccaaaatgtcctttaatcttgtggtcttaaatatgtcacgtgaaaaattgaagttaaagtgttgccaaaaaaagaaaggggtcattcttttttaaacaaactaaaaaagaaaactaagtcattccttttgaaacggaggaagtatactatatttatatatttggttctcgcaaacctgcaaataagggcggatgcacaacagaagttacgagtttgattaaatcttctaattttagtttaaactttgtatatatgcgtagagaagtcataaagttcaaatactgaatttactaattttatcaaagttgtgatctactgctgattaaagttgggtgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaatgtcaaaagttgcattcttctcaaagggcaattcagactttcatcactccctagtgagggcgcggagctagatatgcaaagggtttatataaatttatgtgatacattttttttagttaatctaaaaaataataaaagctttttttttttaccaagatcgatttgaaatcacacaaatatttgtgaatttagatcataaatttcaaaagtcttttaataaaaggctaccttctgttcttggccttttagaggttaggtacatgctccatcccctcctttcttgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacattcttttacttgatttattaaatttagatatatcttcgatctgccacataacataacaaatggtttcaaacggaacatatgactcttaataaaaggtggagaagggttgaaaactttactaaatttaacaagatcttaggtgtgtatttgactcatgttgcaagattgaagtgtatttaagttcagttaatcaagtaaatagatatttttaaggttgtcaatatataattcggagataaaactagccttttgtttcatttgttgagtttaaaattaagttgatatgaaacaacaaataaattgtgatcccaagttttatatgagcaaaataaaatcaagcattgtatatctctcttttatttttctccctggtatccgcattggagcctgactatattcgaatcgcactataggcttcatttttttcatactcgaggcttaaatctgaaacctctgggtaaggatagaatatcttatatctctttttttttttttttggattttccattcaacgtttggtacgcaaattgaagttcagttaattcaaatttgcaccgcataggctaagcggtttctaccaatgatcttttcatacccagggactcaaacacaagacgtttaattaagaagtaaatagtctcatccgctataccacattgtgtggtgatatatctcattaagataccacattatatctatttaagggggtaggggggatgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctctcactcacacagctagaatatatatatatatatatatatatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctaccaactttaattttcttcttattgcagctagcttgcttaattagcagcaaaactcaaaagaggttttagctgtgtttatactgtctttctcaagatctagacccaccacttagaccatctcaagctacagctactgcgcaa

SEQ ID NO: 102 - DNA, 3000 bp, unidentified
  source 1..3000: mol_type = other DNA; note = the promoter regions of IT1 nucleotide sequences
gttttatatcacaaattttttaggttgccgatccagagaatttaaatattaatgttgtgaattctaatactccacctgttttaaaaaatggtctagtttgacttgaaacggagtttaagaaaagaaaaaagactttctaatcttgtgattccaaattaaagttatgtcaaatgtaccaaaatgtcctttaatcttgtggtcttaaacatgtcacgtgaaaaattgaagttaaagtgttgccaaaaaaagaaaggggtcattctttttgaaacaaactaaaaaagaaaactaagtcattccttttgaaacggaggaagtatactatatttatatatttggttctcgcaaacctgcaaataagggcgaatgcacaacagaagttacgagtttgattaaatcttctaattttagttaaaactttgtatatatgcgtagagaagtcataaagttcaaatactgaatttactaattttatcaaagttgtgatctactgctaattaaagttgggtgggcaggtggggatgcccggtattggggaatgtagtatactgtatatctaaaagcaattttggcaaaaccatctctgtcactgatctctcacttcacaatactaacatatagagctggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggcgaggagctagatatgcaaagggtttatataaatttatgtgatacattttttttagttaatctaaaaaaaagaataaaagttttttttttttaccaagatcgatttgaaatcacacaaatatttgtgaatttagatcataaatttcaaaagtcttttaataaaaggctagcttctgttcttagccttttagaggttaggtacatgctccatcccctcctttattgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcaatttggtataaaccaatagtttcgtcaatctaaaaaatattcttttacttgactcactaaatttagatatatcttcgatctgccacataacataacaaatgatttcaaacttttgtcggaacatatgacttaataaaaggtggagaagggttgaaaactttactaaatttaacaagatcctaggtgtgtatttgactcatgttgcaagattgaagtgtatttaaatttagttagtcaagtaaagatatatttttaaggttgtcaatatataattcagaaataaaactaataattcacactgaatttagggggtgttttcaatactcttcttttgtttcatttcttgagtttaaaattaagttgatacggaacaacaaataaattgtgatcccaagttttatatgagcaaaataaaatcaagcattgtatatctctcttttatttttctccctggtgtccgcattgttgcccgactatattcgattcgcactataggcttcattttttttcatactcagggcttaaatctgaaatctgtgggtaagggtagaatatcttatatctctttttttttttttttatagattttccatccaacgtttgttacccacattgaagttcaattaattcaaatttgcgccgcataagctaagcggttcctaccaatgatctttccatacccagggactcaacgcaaaacatttatttaagaaataaatagtctcatccgctatatagtgtatctcattaagataccacattatatctatttaagggggtaggggggatgggtctagctagggttgtggaaatgaggttagagagagggtggtttttcagtgtgtgtgtcaggtgaaatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctctcactcacacagctagaatatatatattcatagttagcagaagtacttttcatataccaaaaaccatacaaatacattgtatcaagtgctgtcatactcaagcaaaagaaagaaaagaacaagatatagtactaccgttttcatcaccattttggtcaatcatgatgattctgaacaaagatatagtactagctaggtagaaaataaatctactaactttaattttcttcttattgcagctagcttgcttaattagtagcaaaatcaaaagaggttttagctgtgtttatactgtctttctcaagatctagacccaccacttagaccatctcaagctacagctactgcgcaa

SEQ ID NO: 103 - DNA, 3000 bp, unidentified
  source 1..3000: mol_type = other DNA; note = the promoter regions of IT1 nucleotide sequences
attttttaggttgccgatccagagaatttaaatattaatgttgtgaattctaatactccacctgttttaaaaaatggtctagtttgacttgaaacagagtttaagaaaagaaaaaagactttctaatcttgtgattccaaattaaagttatgtcaaatgtaccaaaatgtcctttaatcttgtggtcttaaacatgtcacgtgaaaaattgaagttaaagtgttgccaaaaaaagaaagggttcattctttttgaaacaaactaaaaaagaaaactaagtcattccttttgaaacggaggaagtatactatatttatatatttggttctcgcaaacctgcaaataagggcggatgcacaacagaagttacgagtttgattaaatcttctaattttagtttaaactttgtatatatgcgtagagaaagtcataaagttcaaatactgaatttactaattttatcaaagttgtgatctactgctgattaaagttgggtgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggcgcggagttagatatgcaaagggtttatataaatttatgtgatacattttttttagttaatctaaaaaataataaaagcttttttttttaccaagatcgatttgaaatcacacaaatatttgtgaatttagatcataaatttcaaaagtcttttaataaaaggctagcttctgttcttggccttttagaggttaggtacatgctccatcccctcctttcttgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacattcttttacttgacttactaaatttagatatatcttcgatctgccacataacataacaaatggtttcaaacggaacatatgactcttaataaaaggtggagaagggttgcaaactttactaaatttaacaagaccttaggtgtgtatttgactcatgttgcaagattgaagtgtatttaagtttagttagtcaagtaaagatgtatttttaaggttgtcaatatataattcgaagataaaactaataattcatgctgaatttaggaggtgttttcaatacttttcttttgtttcatttgttgagtttaaaattaagttgatatgaaacaacaaataaattgtgatcccaagttttatatgagcaaaataaaatcaagcattgtatatctctcttttatttttctccctggtatccgcattggagcccgactatattcgaatcgcactataggcttcatttttttcatactcgaggcttaaatctgaaacctctgggtaagggtagaatatcttatatctctttcttttctttttttggattttccattcaacgtttggtacccacattgaagttcagttaattcaaatttgcgccgcacaggctaagcggttcctaccaatgatcttttcataccaagggactcaaacacaagacgtttaattaagaaataaatagtctcatccgctatagcacattgtgatatatctcattaagataccacattatatctatttaagggggtaggggggatgggttagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgctattcttccttcctttccccttcccctatattcttcctttatctctctttcacaataatagcaccatgcctgtagcttgatgcttagacgggtgcacacgcactctctcactcacacagctagaatatatatatatatatatatatatattcatagttagcagaagtacttatcatataccaaaaaccacacaaatacattgtatcaagtgctgtcatactcaagcaaaagaaagaaaagaacaagatatagtactactgtttcatcaccaatttggtcaatcatgatgattctgaacaaagatatagtactagctaggtagaaaataaatctactaactttaattttcttcttattgcagctagcttgcttaattagtagcaaaatcaaaagaggttttagctgtgtttatactgtctttctcaagatctagacccaccacttagaccatctcaagctacagctactgcgcaa

SEQ ID NO: 104 - DNA, 3000 bp, unidentified
  source 1..3000: mol_type = other DNA; note = the promoter regions of IT1 nucleotide sequences
gattcggagggaaaatcatgttttatatcacaaattttttaggttgccgatccagagaatttaaatattaatgttgtgaattctaatactccacctgttttaaaaaatgatctagtttgacttgaaacggagtttaagaaaagaaaaaagactttctaatcttgtgattccaaattaaagttatatcaaatgtaccaaaatgtcctttaatcttgtggtcttaaatatgtcacgtgaaaaattgaagttaaagtgttgccaaaaaaagaaaggggtcattcttttttaaacaaactaaaaaagaaaactaagtcattccttttgaaacggaggaagtatactatatttatatatttggttctcgcaaacctgcaaataagggcggatgcacaacagaagttacgagtttgattaaatcttctaattttagtttaaactttgtatatatgcgtagagaagtcataaagttcaaatactgaatttactaattttatcaaagttgtgatctactgctgattaaagttgggtgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaatgtcaaaagttgcattcttctcaaagggcaattcagactttcatcactccctagtgagggcgcggagctagatatgcaaagggtttatataaatttatgtgatacattttttttagttaatctaaaaaataataaaagctttttttttttaccaagatcgatttgaaatcacacaaatatttgtgaatttagatcataaatttcaaaagtcttttaataaaaggctaccttctgttcttggccttttagaggttaggtacatgctccatcccctcctttcttgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacattcttttacttgatttattaaatttagatatatcttcgatctgccacataacataacaaatggtttcaaacggaacatatgactcttaataaaaggtggagaagggttgaaaactttactaaatttaacaagatcttaggtgtgtatttgactcatgttgcaagattgaagtgtatttaagttcagttaatcaagtaaatagatatttttaaggttgtcaatatataattcggagataaaactagccttttgtttcatttgttgagtttaaaattaagttgatatgaaacaacaaataaattgtgatcccaagttttatatgagcaaaataaaatcaagcattgtatatctctcttttatttttctccctggtatccgcattggagcctgactatattcgaatcgcactataggcttcatttttttcatactcgaggcttaaatctgaaacctctgggtaaggatagaatatcttatatctctttttttttttttttggattttccattcaacgtttggtacgcaaattgaagttcagttaattcaaatttgcaccgcataggctaagcggtttctaccaatgatcttttcatacccagggactcaaacacaagacgtttaattaagaagtaaatagtctcatccgctataccacattgtgtggtgatatatctcattaagataccacattatatctatttaagggggtaggggggatgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctctcactcacacagctagaatatatatatatatatatatatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctaccaactttaattttcttcttattgcagctagcttgcttaattagcagcaaaactcaaaagaggttttagctgtgtttatactgtctttctcaagatctagacccaccacttagaccatctcaagctacagctactgcgcaa

SEQ ID NO: 105 - DNA, 3000 bp, unidentified
  source 1..3000: mol_type = other DNA; note = the promoter regions of IT1 nucleotide sequences
atgggagaaacctttatttctaatagtgcgtgctgttacgttgccgatcagagaatttaaatattaatgttgtgaattctaatattccctccgtttaaaaaagaatggtttagtttaacttggaacggagtttaacaaaagaaagaaaactttttaatcttatggttctaaattaaagttatgtcaaatgtaccaaaatgctctttaatcttgtggtcctaaacatgtcacgtgataaattgaagctaaagtgttgccaaaaaaagatagggatcattcttttttaaataaacaaaaaaagaaactaagtcattttttttttaaatggagaaaatatattatatttatatatttggttctcgcaatcctgcaaataagggcggatgcgcaatagaagttacgagtttgattaaatctactaaatttattttaaactttgtatatatgtgtagagaagtcatatagttcaaatactgaatttactgattttatcgaagttgtgatctactgctaattaaagttgggtgggcaggtgaggatacccggtattgaggaatattgtatactgtacatctaaaagcaatttaggcaaaaccatctctgtcactgatctctcacttcacaatactaacagatagagttggggccaaagtagtcatgtagttgatgggaagagatagtacggaggaatggagaaaaaggtaggtgggtgagttgggggaagagatagggttctctttttagtgaaagtgacatcctctaaggtcaaaagttgcattcttctcaaagggcaattcagactttcatcactccctagtgatggcgtggagctaaatatgcaaggggtttatatagatttatgtgatatattttttttaattaatctaaaaaagaatataagttcttttttttaccaagatcaatttgaaatcacacaaatatttgtgaatttatattataaatttcaaaagtcttttaataaaaggctagcttctgtttttggccttttagaggttggtacatgctccatcccctccttccttgctgttactatttgccatggccggtagggatcaaggtcaaaccccaatgcatcgcaggtcaatatagcccgaacagtgacaattttctagagggggattgtgtagaatcaaatatctattcataaattgagacgaatattcttctttacattattgagacgaaaaaaatattaaattttcttaataaaaatcttagctcctcactatttagtctaggtttcatattcattgtattgctgggctttatgttattacacaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttgcgacctagcctttctttttaagaagtataaaaaacatacatcaatttggtataaatcaatagtttcttcaatcttaaaagtatttttttagttgactcactagatatatcttcaatctatcacataacatagcaaatgatttcaaatttttgtcgaaacatatgactcttaataaaaggtggagaagggttgaaaactttactaaatttaacaagatcttagaggtgtatttgactcatgttgtaagattgaagtgtatttaagttcagttaatcaaataaacgggtaattttaaggttgtcaacatataattcggagatgaaactaataattcacactaaatttaggggatgttttcgatacttttcttttgtttcatttgttgagtttaaaattaagttgatatgaaacaacaaataacttgtgatcccaagttttatatcagcaaaataaaatcaagcattgtatatctctcttttatttttctccctggtgtccgcattggagcccgactatattcaaatcacactataggcttcatttttttcatactcagagcttaaatctgaaatctctaggtaagggtagaatatcttatatctctttcttttttcttttttttttttgaattttccatccaacgtttgatacccaccttgaagttcagttaattcaaatttgcgccccataggctaagcaattcctaccaatgatctttccatacctagggactcaaacgtcttttaagaaataaataatctcatccgctataccatatcacttaatagcgtggtataataccacattatatctatttaagggggtagggggatgggtctagctagggttgtggaaaggaggttagagagagagaggtttttcagtgtgtgtgcgtgtcaggtgatatcacgtgaaggggcacgataacaagttgttcgtatgtattcacttccaagaataccgctacgtaccactagctacttccttcctatccgcatctttgtcaaatttcctttccttttccaattactttttgttttatagacatggacgcatttcttccttcctttccccttcccctatattcttcttcctttatctctctttcacaatcatagcaccatgcctgtagcttgacggtgcacgcgcactctcactcacacagctagaatatatatattcatagttagcagaagtacttatcatataccaaaaaccacacaaatacattgtatcaaatgctgtcatactcaagcaaaagaaagaaaagaacaagatatagtacaactgttttcatcaccatttcggtcaatcatgatgattctgaacaagatatagtactagctaggtagaaaataaatctactaactttaattttcttcttattgcagctagcttgcttaattagcagcaaaatcaaaagaggttttaactgtgtttatactgtctttctcaagatctagacccaccacttagaccatctcaagctacagctactgcgcaa

SEQ ID NO: 106 - DNA, 3000 bp, unidentified
  source 1..3000: mol_type = other DNA; note = the promoter regions of IT1 nucleotide sequences
attgaatgtttaagaaagaaaatcaaataattttcgtcaatttattttcatgcaaaaatacagagaaatatcaaaaaaataatattatttgtttgaaattttttatgttcgttcgcgaatgtttaattttttaattaacaggcacacatctattttttaaaaataagaaattgcaattgaagaacataaataaaaataaatcataaaaattagtataagtgtttaaatggtacataaaaaatgaagtttaagaattaaaatggaacaagactaagttaaagtgccaaaataaaaaataaggacaaatttgaggggctgtatatgtgtttgggcttttatcaaagttgtgatctactgctaattaaagttgggtgggcaggtgaggatgcctggtattggggaatatagtatactgtacatctaaaagcaattgaggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagaccttcatcactccctagtgagggcgcggagctagatatgcaaggggtttatataaatttatgtgatacattttttttaagttaatctaaaaagcaataaaagtttttttttttaccaagattgatttgaaatcacacaaatatttataaatttagattaataaaaggctaacttctgttcttggccttttagaggttaggtacatgctccatcccctccttccttgttgttactatttgccatggccggtagggatcaagtcaaaccccaatgcatcacatgccagtatagtccgaacggtgacagttttctaggagggattgtgtagaatcaaacactaaaagaaaaatgtaaattacatgagaatttttctagggattaatataaaatttatagaaaaataagttttctacagattttcatactaatccctcaaaaaaattctcatgtaattcacactttttttatagtgaaatatctattcataaattgagacattgagacgaatattcttctttgcattattgagacgaagaaaatattaaattttctttataagaatcttagctcctcactatttagtctaggtttcatattcattgaattgctgttattactcaactgaaataaaaagagaattgtcgttatccaggttagacaaaagcacgtgtagtttcaaacgcagtagccattattcccctctcctttacaggtgagattcaagggcagaaaggtaccatgcaagctgttatttaaatttacttattgattatgatcgacgactttgacagaaaaatgatgatgctttaattttgcgacctagcctttctttttaagaagtataaaaatcatacctcaatttggtataaatcaatagttttgtcaatcttaaaaagcatttttttacttgactcactaaatttaaatatatcttcgatatgtcacatttcaaactcttgtcgaaacatgacttttaattaaaggtggagaagggttgaaaactttactaaatttaacaagatcttaggggtgtattcgactcataatgcaagattgaagtgtatttaagttcagttagtcaagtaaagggatatttttaaggttgtcaatatataattcggagataaaactaataattcacactgaatttagggggtgttttcgatacttttcttttgtttcatttgttgagtttaaaattaagttgatatgaaacaataaataaattgtgaccccaagttttatatcagcaaaataaaatcaagcattgtatatctctcttttatttttctccctggtgtccgcattggaacccgattatattcaaatcgcactataagcttcatttttttcatactgaggacttaaatctgaaacctctagataagggtagaatatcttatatctctttctttattcttgttttttggattttccatccaacgtttggtatccacattgaagtttagttgattcaatttgcaccgcataggctaagcggttcccaccaatgatctttccatacccagaaactcaaacacaaaacgtttgtttaagaaataaatagtaccatcggctataccacatcacttgatagtgtggtatatctcattaagataccagattatatctatttaagggggtggggtggggtggggtggcgtgcggggatgggtctagcaagagttgtggaaaggaggttagagagagagtggtttttcagtgtgtgtgtcaggtgatatcacgtgaagcacgataacaagttgttcatatccattcacttccaagaataccgctacgtaccacttgctacttccttcctatccccatctttgtcaaatttcctttccttttccaattactttttctttaatagacatggacgcaattcttccttcctttccccttcccctatattcttcctttaactctctttcacaataatagcaccatgcctgtagcttgatgcttagacgggtgcacacacactctcactcacacagctagaatatatatattcatagttagcacaagtacttatatcatatataccaaaaaccacacaaatacattgtatcaagtgctgtcatactcaagcaaaaaaaaaaagaaaagaacaagatatagtactactgttttcatcaccattttggtcaatcatgataattctgaacaagatatagtactagctagctaggtagaaaataaatctactaactttaattttcttcttattgcagctagcttgcttaattagcagcaaaatcaaaagatgtgttagctgtgtttataccgtctttctcaagatctagacccaccacttagaccatctcaagctacagctactgcgcaa

SEQ ID NO: 107 - DNA, 3000 bp, unidentified
  source 1..3000: mol_type = other DNA; note = the promoter regions of IT1 nucleotide sequences
aagaatggtctagtttaacttgaaacggagtttaacaaaagaaaaaagactttttaatcttgtggttctaaaataaagttgtgtcaaatgtaacaaaatgctctttaatcttgtggtcctaaacatgtcacatgaaaagttgaagctaaagtgttgccaaaaaaagaaagagatcattctttttgaaacaaacaaaaaaggaaactaagtcattttttttaaaaggaatgaagtatacaatatttatatatttggttctcgcaaacctgcaaataagggcggatgcacaatagaagttacgagtttgattaaatatactaattttagtttaaattttgtatatatgtgtagagaagtcataaagttcaaatactgaatttactaattttatcaaagttgtgatctactgctaattaaagttgggtgggcaggtgaggatgcctggtattggggaatatagtatactgtacatctaaaagcaattgaggcaaagccatctctgtcactgatctctcacttcacaatactaacaaatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagaccttcatcactccctagtgagggggcggagctagatatgcaaggggtttatataaatttatgtgatacatttttttttagttaatctaaaaaagaataaaagttttttttttttttaccaatttgaaatcacacaaatatttataaatttagatcataaatttcaaaagtcttataataaaaggctagcttatgttcttagcattttagaggttaggtacatgctccatcccctccttccttgttgttactatttggcatggctggtagggatcaaggtcaaaccccaatgcatcacaagtcagtatagtccgaccggtgacaattttctaggggggatcgtgtagaatcaaacactacaagaaaaatgtgaattacatgagaattttttagggattaatatgaaaatttgcagaaaaataagttttctacagattttcatactaatctccaagaaaattctcatgtaattcacactttttttatagtgaaatatctattcataaattgagacattgagacgaatattctactttacattattgagacaaagaaaatattaataagaatcttagctcctcactatttagtctaggtttcatattcattgaattgctggggtttatgttattactcaactgaaataaaaagagaattgtcgttatccaggttagacaaaagcacgtgtagtttcaaacgcagtagccattattcccctctcctttacaggtgagattcaagggcagaaaggtaccatgcaagctgttatttaaatttacttattgattatgatcgacgactttgacagaaaaatgatgatgctttaattttgcgacctagcctttctttttaacaagaataaaaaacatacctcaatttggtataaatcaatagtttcgtcaatcttaaaagcatttttttacttgactcactaaatttaaatatatcttcgatatgccacatttcaaactcttgtcgaaacatatgactgaagggttgaaaattttactaaatttaacaagatcttaggggtgtatttgactcataatgcaagattgaagtgcatttaagttcagttagtcaagtaaagagatatttttaaggttgtcaatatataattcggagataaaactaataattcacactgaatttagggggtgttttcgatacttttcttttgtttcatttgttgagtttaaaattaagttgatatgaaacaacaaataaattgtgatcccaagttttatatcagcaaaataaaatcaagcatcgtatatctctcttttatttttctccctggtgtctgcattggaacccgactatattcaaatcgcattataggctttatttttttcatacttagggcttaaatctgaaacctctagatcagggtaaaatatcttatctctttttttttttctccatccaacgtttgatatccacattgaagttcaattgatttaaatttgcaccgcataggctaagcggttcctaccaatgatctttccatacccagaaactcaaacgcaaaacatttgtttaagaaataaatagttccatccgctataccacatcacttgatagtgtggtatatctaattaagataccacattatatctatttaagggggtagggtgggggatgggtctagctaggtttgtggaaaggaggttagagagagagtggtttttcagtgtgtgtgtcaggtgatatcacgtgaaggggcacgataacaagttgttcatatccattcacttccaagaataccgctacgtaccactagctgcttccttcctatccccatctttgtcaaatttcctttccttttccaattactttttctttaatagacatggacgcaattcttccttcctttccccttcccctatattcttcttcctttatctctctttcacaataatagcaccatgcctgtagcttgacactctcactcacacagctagaatatatatatagttagcagaagtacttatcatatataccaaaaaccacacaaatacattgtatcaagtgctgtcatactcaagcaaaaaaaagaaaagaacaagatatagtactactgttttcatcaccatttgggtcaatcatgataattctgaacaagatatagtactagctagctaggcagaaaataaatctacatttgtttaattttcttcttattgcagctagcttgcttaattagcagcaaaatcaaaagaggttttagctgtgtttataccgtctttctcaagatctagacccaccacttagaccatctcaagctacagctactgcgcaa

SEQ ID NO: 108 - DNA, 3000 bp, unidentified
  source 1..3000: mol_type = other DNA; note = the promoter regions of IT1 nucleotide sequences
tccagagaatttaaataataatgttgtgaattctaatactccacctgttttaaaaaatagtctagtttgacttggaacggagtttaagaaaagaaaaaagattttctaatcttatggttccaaattaaagttatgtcaaatgtaccaaaatgccctttaatcttgtggttctaaacatatcacgtgaaaacttgaagctaaagtgttgccaaaaaaagaaagggatcattctttttgaaacaaacaaaaaagaaaactaagtcatttttttttaaacggagaaagtatactatatttatatatttggttctcgcaaacctgcaaataagggcggatgcacaacagaagttacgagtttgattaaatctactaattttagtttaaactttgtatatatgtgtagaggagtcataaagttcaaatactgaatttactaattttatcaaagttgtgatctgctgctaattaaagttgagtgggcaggtggggatgcccggtattggggaatgtagtatactgtacatctaaaagcaattttggcaaaaccatctctgtcactgatctctcacttcacaatacttaacatatagagttggggccaaagtagtcatgtagttgatgggaagagatagtacggaggaatggagaaaaaggtaggtgggtgagttgggggaagagatagggttctctttttagtgaaagtgacatactctaaggtcaaaagttgcattcttctcaaagggcaattcagaccttcatcactccctagtgagggcgcggagctagatatgcaaagggtttatataaatttatgtgatacatttcttttagttaatctaaaaaaagaataaaagtcttttttttaccaagatcgatttgaaatcacacaaatatttgtgaatttagatcataaatttcgaaagtcttttaataaaaggctaacttctgttcttggccttttagaggttaggtacatgctccatcccctcctttcttgttgttactatttgccatggccggtagggatcagggtcaaaccccaatgtatcacaggtcaatatagtccgaccgtgacaattttctagggggattgtgtagaatcaaatatctattcataaattgagacattgagacgaatattcttctttacattattgagacgaagaaaatattaaattttcttaataaaaatcttagctcctcactatttggtctaggtttcatattcaatgaattgctggggtatatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactgtgacagaaaaatgatgatgctttaattttacgacctagcctttctttttaagaagaataaaaaacatacctcaatttggtataaaccaatagtttcgtcaatcttaaaaacattattttatttgactcactaaatttagatatatcttcaatctgccacataacataacaaatgatttcaaactcttgtcggaacatatgactctttataaaaggtggagaagggttgaaaactttactaaatctaacaagatcttaggtgtgtatttgactcatgttgcaagattgaagtgtatttaaattcagttagtcaagtaaagaaatatttttaaagttgtcaatatataattcggagataaaataataattcacactggatttagagggtgttttcgattcttttcttttgtttcatttgttgagtttaaaattaagttgatatgaaacaacaaataaattgtgatcccaagttttatatgagcaaaataaaatcaagcattgtatatctctcttttatttttctctctagtgtccgcattggagctagactatattcgaatcgcactataggcttcatttttttcatacttgaggcttaaatctgaaacctctgggtaagggtagaattatcttgtatctctttcttttctttctttggattttccatccagcgtttggtacccacattgaagtttagttaattcaaatctgcaccacataggctaagcggttcctaccaatgatctttccatacctagggactaaaacacaagacgtttaattaagaaataaatagtcccatccactataccacatcagttgatagtgtggtatatctcattaagataccacattatatctatttaagggggtaggggggacgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcatccttcctttccccttcccctatattcttcctttatctctctttcacaataatagcaccatgcctgtagcttgatgcttagacgggtgcacacgcactctcactcacacagctagaatatatatatatatatatatatatatatatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagttactagctaggtagaaaataaatctactaactttaattttcttcttattgcagctagcttgcttaattagcggcaaaactcaaaagaggttttagctgtgtttatactgtctttctcaagatctagacccaccacttagaccatctcaagctacagctactgcgcaa

SEQ ID NO: 109 - DNA, 3000 bp, unidentified
  source 1..3000: mol_type = other DNA; note = the promoter regions of IT1 nucleotide sequences
tagcacaagatttttaggttgccgatgcagagaatttaaatattaatgttgtgaattccaatactccacccgttttaaaaaatggtctagtttgactagaaacggagtttaagaaaaaaaaagaccttctacttgtggtttcaaattaaagttatgtcaaatgtaccaaaatgtcctttaatcttgtggtcctaaacatatcacgtgaaaagttgaagctaaagtgttgcaaaaaaagaaagggattattcattttgacacaaacaaaaaagaaaactaattcattctttttgaaacggaggaatcatactatatttatatatttggttctcacaaacctgcaaataagggcggatgcacaacagaagttacgagtttgattaaatctactaattttagtttgagctttgtatatatgtgtagaggagtcataaagttcaaatattgaatttactaattttatcaaagttgtgatctactgctaattaaagttgggtgggcaggtggggatgcccggtattggggaatgtagtatactgtacatctaaaagcaattttggcaaaaccatctctgtcactgatctctcacttcacaatactaacatatagagctggggccaaagtagtcatgtagttgatgggaagagatagtacggaggaatggagaaaaaggtaggtgggtgagttggggaagagatagggttctctttttagtgaaagtgacatgctctaaggtcaaaagttgcattcttctcaaagggcaattcagactttcatcactccctagtgagggcgaggagctagatatgcaaagggtttatataaatttatgtgatacatttttttttagttaatctaaaaaaaaagaataaaagttttttttttacaccaagatcgatttgaaatcacacaaatatttgtgaatttagatcatgaatttcaaaagtcttttaataaaaggctagcttctgttcttagccttttagaggttaggtacatgctccatcccctcctttcttgttgttactatttgccatggccgatagggatcaaggtcaaaccccaatgcatcatagcagtatagttcgaccgtgacaattttctatgggggattgtgtagaatcaaatatctattcataatttgagacattgagacgaatattcttctttacattattgagacaaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagcaattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcaatttggtataaaccaatagtttcgtcaatctaaaaaatattcttttacttgactcactaaatttagatatattgccacataacataacaaattgtttcaaacttttgtcggaacatatgactcttaataaaaggtggagaagggttgaaaactttactaaatttaacaagatcttatgtgtatatttgactcatgttgcaagattgaagtatatttaaatttagttagtcaaggtatttttaaggttgtcaatatataattcgaagataaaattaataattcacactgaatttagggagtgttttcaatacttttcttttgtttcatttcttgagtttaaaattaagttgatatgaaacaacaaataaattgtgatcccaagttttatatgagcaaaataaaatcaagcattgtatatctctcttttatttttctccctagtgtccgcattggagcccgactatattcgaatcgaactataggcttcatttttttcatactcagggcttaaatctaaaacctgtgggtaagggtagaatatcttatatctctttttttttttttttcttttttccatccaacatttgttacccacattgaagttcaattaattcaaatttgcgccgcataggctaagcggttcctaccaatgatctttccatacccagggactcaaacgcaaaacatttatttaagaaataaatagtctcattcgctataccacatcagttgatagtgtgatatatctcattaagataccacattatatctatttaagggggtaggggggacgggtctagctagggttgtggaaatgaggttagagagagggtggtttttcagtgtgtgtgtcaggtgaaatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctcactcacacagctagaatatatatatgaatatatatattcatagttagcagaagtacttatcatataccaaaaaccatacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctactaactttaattttcttcttattgcagctagcttgcttaattagtagcaaaatcaaaagaggttttagctgtgtttatacggtctttctcaagatctagacccaccacttagaccatctcaagctacagctaccgcgcaa

SEQ ID NO: 110 - DNA, 3000 bp, unidentified
  source 1..3000: mol_type = other DNA; note = the promoter regions of IT1 nucleotide sequences
gtggttctaaattaaagttgtgtcaaatgtatcaaaatgccctttaattttgtggtcctaaacatgtcacatgaaaagttgaagctaaagtgttgtcaaaaaaagaaaagggtcattctttcttaaacaaacaaaaaaagaaactaagtcattctttttaaaacgaagggagtatactatatttatatatttggttctcacaaacctgcaaataagggcggatgcacaatagaagttatgagtttgattaaatctactaattttagtttaaactttgtatatatgtgtagaggagtcataaagttcaaatactaaatttactaattttatgaaagttgtgatctactgctaattaaagttgggtgggcatgtgaggatgcctggtattggggaatatagtatactgtacatgtaaaagcaattgaggcagaaccatctctgtcactgatctctcacttcacaatactaacaaatagagttggggccaaagtagtcatgtagttgatgggaagagatagtacggatgaatggagaaaaaggtaggtgggtgagttgggggaagagatagggttctctttttagtgaaagtgacattctctaaggtcaaaagttgaattcttctcaaagggcaattcagaccttcatcactccctagtgagggcgcggagctagatatgcaaggggtttatataaatttatgtgatacattttttttagttaatctaaaaaaagaataatttttttatttttttttaccaagattgatttgaaatcacacaaatatttataaatttagatcataaatttcaagtcttttaataaaaggctagcttctgttcttggccttttagaggttaggtacatgctccatcccctccttccttgttgttactatttgccatggccagtagggatcaaggtcaaaccccaatgcatcacaggtcagtatagtccgaccggtgacaattttataagggggattgtgtagaatcaaacactacaagaaaattgtgaattacatgaaatattttctagagattagtatgaaaatttacagaaaaataagttttctacagattttcatactaatccccaagaaaattctcatgtaattcacactttttttaatagtgaaatatatattcataaattgagacattgagacgaatattcttctttacattattgagacaaagaaaatattaaatttattttaaataagaatcttagctcctcactatttagtctaggtttcatattcattgaattgctggggtttatgttattactcaactgaaataaaaagagaattgtcgttatccaggttagacaaaagcacgtgtagtttcaaacgcagtagccattattcccctctcctttacaggtgagattcaagggcagaaaggtaccatgcaagctgttatttaaatttacttattgattatgatcgacgactttgacagaaaaatgatgatgctttaattttgcgacctagcctttctttttaagaagtataaaaaacatacctcaatttggtataaatcaatagtttcgtcaatcttaaaagcatttttttacttgactcactaaatttaaatatatcttcgatatgccacatttcaaacttatgtcggaatatatgacttttaataaaaggtggagaagggttgaaaactttactaaatttaacaagatcttaggggtgtatttgacatataatgcaagattgaagtgtatttaagttcagttaatcaagtaaaggggtatttttaaggttgtcaatatataattcgtagatgaaactaataattcacactgaatttaggaggtgttttcgttacttttcttttgtttcatttgttgagtttaaaattaagttgatatgaaacaacaaataaattctgatcccaagttttatattagcaaaataaaatcaagcattgtatatctctcttttatttttctccctggtgtccgcattggagtccgactatattcgaatctcactataggcttcatttttttcatactcgtggcttaaatctgaaatctcttgataagggtagaatatcttatatatttttctttttttttgaattttccatccaacgtttgatattcacattgaagttcagttgattcaaatttgcgccgcataggctaagcagttcctactaatgatctttccatacctaggatatcaaacggaaaacgtttgtttaagaaataaatagtttcatccgctataccacatcacttgatagtgtggtatatctaattaagataccacattatatctatttaagggggtagggggggggatgggtctagctaggtttgtggaaaggaggttagagagagagtggtttttcagtgtgtgtgtcaggtgatatcacgtgaaggggcacgataacaagttgttcatatccattcacttccaagaataccgctacgtaccactggctgcttccttcctatccccatctttgtcaaatttcctttccttttccaattactttttctttaatagacatggacgcaattcttccttcctttccccttcccctatattctttcctttatctctctttcacaataatagcaccatgcctgtagcttgatgcttagacgggtgcacacacacactctcactcacacagctagaatatatatatattcatagttagcagaagtacttatcatatataccaaaaaccacacaaatacattgtatcaagtgctgtcatactcgagcaaaaaaaagaaaagaacaagatatagtactactgttttcatcaccattttggtcaatcatgaacaagatatagtactagctagctaggtagaaaataaatctactaactttaattttcttcttattgcagctagcttgcttaattagcagcaaaatcaaaagaggtttaagctgtgtgtttataccgtctttctcaagatctagacccaccacttagaccatctcaagctacatctactgcgcaa

SEQ ID NO: 111 - DNA, 3000 bp, unidentified
  source 1..3000: mol_type = other DNA; note = the promoter regions of IT1 nucleotide sequences
tgattcggtgggaaaatcatgttttatatcacaaattttttaggttgccgatccagagaatttaaatattaatgttgtgaattctaatactccacctgttttaaaaaatggtctagtttgacttgaaacggagtttaagaaaagaaaaaagactttctaatcttgtgattccaaattaaagttatatcaaatgtaccaaaatgtcctttaatcttgtggtcttaaatatgtcacgtgaaaaattgaagttaaagtgttgccaaaaaaagaaaggggtcattctttttgaaacaaactaaaaaagaaaactaagtcattccttttgaaacggaggaagtatactatatttatatatttggttctcgcaaacctgcaaataagggcggatgcacaaaagaagttacgagtttgattaaatcttctaattttagtttaaactttgtatatatgcgtagagaagtcataaagttcaaatactgaatttactaattttatcaaagttgtgatctactgctgattaaagttgggtgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggcgcggagctagatatgcaaagggtttatataaatttatgtgatacattttttttagttaatctaaaaaataataaaagcttttttttttaccaagatcgatttgaaatcacacaaatatttgtgaatttagatcataaatttcaaaagtcttttaataaaaggctagcttctgttcttggccttttagaggttaggtacatgctccatcccctcctttcttgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacattcttttacttgatttattaaatttagatatatcttcgatctgccacataacataacaaatggtttcaaacggaacatatgactcttaataaaaggtggagaagggttgaaaactttactaaatttaacaagatcttaggtgtgtatttgactcatgttgcaagattgaagtgtatttaagttcagttaatcaagtaaatagatatttttaaggttgtcaatatataattcggagataaaactagccttttgtttcatttgttgagtttaaaattaagttgatatgaaacaacaaataaattgtgatcccaagttttatatgagcaaaataaaatcaagcattgtatatctctcttttatttttctccctggtatccgcattggagcccgactatattcgaatcgcactataggcttcatttttttcatactcgaggcttaaatctgaaacctctgggtaagggtagaatatcttatatctctttttttttttttggattttccattcaacgtttggtacccacattgaagttcagttaattcaaatttgcaccgcataggctaagcggttcctaccaatgatcttttcatacccaaggactcaaacacaagacgtttaattaagaagtaaatagtctcatccgctataccacattgtgtggtgatatatctcattaagataccacattatatctatttaagggggtaggggggatgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctctcactcacacagctagaatatatatatatatatatatatatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctactaactttaattttcttcttattgcagctagcttgcttaattagcagcaaaactcaaaagaggttttagctgtgtttatactgtctttctcaagatctagacccaccacttagaccatctcaagctacagctactgcgcaa

SEQ ID NO: 112 - DNA, 3000 bp, unidentified
  source 1..3000: mol_type = other DNA; note = the promoter regions of IT1 nucleotide sequences
cagagaatttaaatattaatgttgtgaattctaatactccacctgttttaaaaaatggtctagtttgacttggaacggagtttaagaaaagaaagaaaactttctaattttgtggttccaaattaaagttatgtcaaatgtacctaaataccctttaattttgtggtcttaaacatgtcacgtgaaaaattgaagtcaaagtgttgtcaaaaaaggaaaggagccattctttttgaaacaaactaaaaaaaaaaactagatcatttttttttaaacggagaagtatactatatttatatatttggttctcgcaaacctgcaaataagggcggatgcacaacagaaattacgagtttgattaaatctactaattttagtttaaactttgtatatatgtgtagaggagtcataaagttcaaatactgaatttactgattttatcaaagttgtgatctactgctaattaaagttgggtgggcaggtggggatgccctgtattggggaatgtagtatactgtacatctaaaagcaattttggcaaaaccatctctgtcactgatctctcacttcacaatactaacatatagagttggggccaaagaagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagaccttcatcactccctagtgagggcgcggagttagatatgcaaaggatttatataaatttatgtgatacattttttttagttaattttaaaaagaaaaaaagattattttttttaccaagatcaaattgaaatcacacaaatatttgtgaatttagatcataaatttcaaaaatcttttaataaaaggttagcttctgttcttggccttttagaggttaggtacatgctccatcccctcctttcttgttgttactatttgtcatggccggtagggatcaaggtcaaaccccaatgcatcacaagtcagtatagtccgaccgtgacaattttctatgggggattgtgtagaatcaaatatctattcataaattgagacattgagacgaatattcttctttacattattgagacgaaaaaaatattaatttttttaaaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttgcgacctagcctttctttttaagaagaataaaaaacatacctcgatttggtataaatcaatagtttcgtccatcttaaaaacattcttttacttgagtcactaaatttagatatttctttgatctgccacataacataacaaatgatttcaaacttttgtcggaacatatgactcttaataaaaggtggagaagggttgaaaactttactaaatttaacaagatcttaggtgtgtatttgactcatgttgcaagattgaagtgtatttaagtttagttagtcaagtaaaacgatatttttaagattgtcaatatataattcggagataaaactaataattcacactgaatttagggagtgttttcaatacctttcttttgtttcatttcttgagtttaaaattaagttgatatgaaacaacaaataaattgtgatcccaagttttatatgagcaaaataaaatcaagcattgtatatctctcttttatttttctccctggtgtccgcattggagtccgactatattcgaatcacactataggcttcatttttttcatactcaaggcttaaatctgaaacctatgggtaagggtagaatatcttatatctctttttttttttcttttggattttccatccaacgtttgttacccacattgaagttcagttaattcaaatttgcgccgcataggctaagcggttcctaccaatgatctttccatacccagggactcaaacgcaaaatatttatttaagaaataaattgtctcatccgctataccacatcagttgatagtgtggtatatctcattaagataccacattatatctatttaagggggtaggggggatgggtctagctagggttgtggaaatgaggttagagagagggtggtttttcagtgtgtgtgtcaggtgaaatcacgtgaaggggcacgataacaagttgttcatatccattcacttccaagaataccgctacgtacagtcactactgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctcactcacacagctagaatatatatatatatattatatatatattcatagttagcagaaatacttatcatataccaaaaaccacacaaatacattgtatcaagtgctgtcatactcaagcaaaaagaaagaaaagaacaagatatagtactactgttttcatcaccattttggtcaatcatgatgattctgaacaaagatatagtactagctaggtagaaaataaatctaccaactttaattttcttcttaatgcagctagctttgcttaattagtagcaaaatcaaaagaggttttagctgtgtttatactgtctttctcaagatctagacccaccacttagaccatctcaagctacagctactgcgcaa

SEQ ID NO: 113 - DNA, 3000 bp, unidentified
  source 1..3000: mol_type = other DNA; note = the promoter regions of IT1 nucleotide sequences
attcggtgggaaaatcatgttttatagcacaaattttttaggttgccgatccagagaatttaaatattaatgttgtgaattctaatactccacctgttttaaaaaatggtctagtttgacttgaaacggagtttaagaaaagaaaaaagactttctaatcttgtgattccaaattaaagttatgtcaaatataccaaaatgtcctttaatcttgtgctcttaaacatgtcacgtgaaaaattgaagttaaagtgttgccaaaaaaagaaaggggtcattattttttaaacaaactaaaaaagaaaactaagtcattccttttgaaacggagaaagtatactatatttatatatttggttctcgcaaacctgcaaataagggcggatgcacaatagaagttacgagtttgattaaatctactaattttaatttaaactttgtatatatgcgtagagaagtcgtaaagttcaaatactgaatttactaattttatcaaagttgtgatctactgctgattaaagttgggtgggcaggtggggatgcccggtattggggaatgtagtatactgtacatctaaaagcaattttggcaaaactatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggcgcggagctagatatgcaaagggtttatataaatttatgtgatacattttttttagttaatctaaaagggaataatttttttttttttaccaagatcgatttgaaatcacacaaatatttgtgaatttagatcataaatttcaaaaatcttttaataaaaggttagcttctgttcttggcctcttagtggttaggtagatgctccatcccctcctttcttgttgttactatttgccatggccggtagggatcaaggtcaaaccccaatgcatcacaggtcagtataatccgaccgtgacaattttctatgggggattgtgtagaatcaaatatctattcataaattgagacattgagacgaatattcttctttacattattgagacgaaaaaaatattaaatttttttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacattcctttacttgacttactaaatttagatatatcttcgatctgccacataacataacaaatggtttcaaacagaacatatgactcttaataaaaggtggagaaggattgaaaactttactaaatttaacaagatcttaggtgtgtatttgactcatgttgcaagattgaagtgtatttaagttcagttaatcaagtaaaaagatatttttaaggttgtcaatatataattcggagataaaactagccttttgtttcatttgttgagtttaaaattaagttgatatgaaacaacaaataaattgtgatcccaagttttatatgagcaaaataaaatcaagcattgtatatctctcttttatttttctccttggtatccgcattggagcccgactatattcgaatcgcactataggcttcatttttttcatactcgagtcttaaatctgaaacctctgggtaagggtagaatatcttatatctctttttttttttttttggattttccattcaacgtttggtacccacattgaagttcagttaattcaaatttgcaccgcataggctaagcggttcctaccaatgatcttttcatacccagggactcaaacacaagacatttaattaagaagtaaatagtctcatccgctataccacattgtgatatatctcattaagataccacattatatctatttaagggggtaggggggatgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcgcctatattcttcctttatctctctttcacaataatagcaccatgcctgtagcttgatgcttagacgggtgcacacgcactctctcactcacacagctagaatatatatatatatatatatatatatatatattcatagttagccgaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctactaactttaattttcttcttattgcagctagcttgcttaattagcagcaaaactcaaaagaggttttagctgtgtttatactgtctttctcaagatctagacccaccacttagaccatctcaagctacagctactgcgcaa

SEQ ID NO: 114 - DNA, 3000 bp, unidentified
  source 1..3000: mol_type = other DNA; note = the promoter regions of IT1 nucleotide sequences
attttttactgatacagatgggagaaacctttatttctagtagtgcgtgctattaggttgccgatccagagaatttaaatattaatgttgtgaattctaacactccctctgtttaaaaaagaatggtctagtttgacttgaaactgagtttaacaaaagaaagaaaactttttaatcttatggttctaaattaaaattatgtcaaatgtaccaaaatgtcctttaatcttgtggtcctaaacatgtcacgtgaaaagttgaagctaaaatgttgccaaaaaaagaaaggggtcattcttttttaaacaaacaaaaaaggaaactaagtcatttttttttaaacgaagggaatatactatatttatatatttggttctcgcaatcctgcaaataagggcggatgcacaatagaagttacgagtttgattaaatctactaattttagtttaaactttgtatatatgtgtagagaagtcataaagttcaaatattgaatttacagattttatcaaagttgtgatctactgctaattaaagttgggtgggcaggtgaagatacccggtattgaggaatattgtatactgtacatctaaaagcaatttaggcaaaaccatctctgtcactgatctctcacttcacaatactaacagatagagttggggccaaagtagtcatgtagttgatgggaagagatagtacggaggaatggagaaaaaaggtaggtgggtgagttgggggaagagatagggttctctttttagtgaaagtgacatcctctaaggtcaaaagttgcattcttctcaaagggcaattcagaccttcatcactccctagtgagggcgcggagctagatatgcaaggggtttatatagatttatgtgatacattttttttaattaatctaaaaaagaataaaagttcttttttttaccaagatcaatttgaaatcacacaaatatttgtgaatttatattataaatttcgaaagtcttttaataaaaggctagcttctatttttggccttttacaggttaggtacatgctccatcccctccttccttgttgttactatttgccatggccggtagggattaaggtcaaaccccaatgcatcacaggtcaatatagcccgaacggtgacaattttctagagggggattgtgtagaatcaaatatctattcataaattgagacgaagaaaatattaaattttcttaataaaaatcttggctcctcactatttagtctaggtttcatattcattgaattgctggggtttatgttattacacaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttgcgacctagcctttctttttaagaagtataaaaaacataccttaatttggtataaatcaatagtttcttcaatcttaaaagtattcttttacttgactcactagatatatcttcaatctgtcacataacataacaaatgatttcaaacttttgtcgaaacatatgactcttaataaaaggcggagaagggttgaaaactttactaaatttaacaagatcttaggggtgtatttgactcatgttgcaagattgaagtgtatttaagttcaattaatcaaataaacggataattttaaggttgtcaacatataattcggagatgaaactaataattcacactgaatttagggggtgttttcgatacttttcttttgtttcatttgttgagtttaaaattaagttgatatgaaacaacaaataaattgtgatcccaagttttatatcagcaaaataaaatcaagcattgtatatctctcttttatttttctccctggtgtccgcattggagcccgactatattcgaatcacactataggcttcatttttttcatactaagggcttaaatctgaaacctctagataagggtagaatatcttatatctctttcttttttctttttttttggattttccatccaacgtttgatacccaccttgaaattcagttaattcaaatttgcgccccataggataagtggttcctaccaatgatctttccatacccagggactcaaatgtcttttaagaaataagtagtctcatccgctataccatagcacttgttagtgtggtatacctcattaagataccacattatatctatttaagggggtagggggatgggtctagctagggttgtggaaaggaggttagagagagagagaggtttttcagtgtgtgtgtgtcaggtgatatcacgtgaagaggcacgataacaagttgttcgtatgtattcacttccaagaataccgctacgtaccactagctacttccttcctatccgcatctttgtcaaatttcctctccttttccaattactttttgttttatagacatggacgcatttcttccttcctttccccttcccctatattcttcttcctttatctctctttcacaataatagcaccatgcctgtagcttgacggtgcacgcgcactctcactcacacagctagaatatatatattcatagttagcagaagtacttatcatataccaaaaaccacacaaatacattgtatcaaatgctgtcatactcaagcaaaagaaagaaaagaacaagatatagtacaactgttttcatcaccattttggtcaatcatgatgattctgaacaagatatagtgtactagctaggtagaaaataaatctactaactttaattttttcttattgcagctagcttgcttaattagcagcaaaatcaaaagagggtttaactgtgtttatactgtctttctcaagatctagacccaccacttagaccatctcaagctacagctactgcgcaa

SEQ ID NO: 115 - DNA, 3000 bp, unidentified
  source 1..3000: mol_type = other DNA; note = the promoter regions of IT1 nucleotide sequences
taaattaaagttgtgtcaaatgtaccaaaatgtcttttaatcttgtggtcctaaacatgtcacatgaaaagttgaagctaaagtgttgtcaaaaaaagaaaggagtcattctttttgaaacaaaaaaaaaaaagaaactaaatcattatttttaaaacaaagggaatatactatatttaaatatttggttctcgcaaacctgcaaataagggcggatgcacaatagaagttacgagtttgattaaatctactaattttagtttaaactttgtatatatgtgtagagaagtcataaagttcaaatactgaatttactaattttatcaaagttgtgatctactgctaattaaagttgggtgggcaggtgaggatggccggtattggggaatatagtatactgtacatctaaaagcaatttaggcagaaccatctctgtcactgatctctcacttcacaatactaacatatagagttggggccaaagtagtcatgtagttgatgggaagagatagtacggaggaatggagaaaaaggtaggtgggggagttgggggaagagatagggttctctttttagtgaaagtgacattctctaaggtcaaaagttgcattcttctcaaagggcaattcagaccttcatcactccctagtgagggcgcagagctagatatgcaaggagtttatataaatttatgtgatacatttttttagttaatctaaaaaagaataaaagatatttattttttacctagattgatttgaaatcacacaaatatttataaatttagatcataaatttcataagtcttttaataaaaggctagcttctgttcttagccttttagaggttaggtacatgctccatcccctccttccttgttgttactatttggcatggccggtagggatcaaggtcaaaccccaatgcatcacatgtcagtatagtccgaccggtgacaattttctaggggggattgtgtagaatcaaacactacaagaaatgtgtgaattacatgagaatttttctaggattaatatgaaaatttgcagaaaaataagtttcctacagattttcatactaatccccaagaaaattctcatgttattcacacttttttttatagtaaaatatctattcataaattgagacattgagacgaatattcttctttacattattgagacaaagaaaatattaaattttcttaataagaatcttagctcctcactatttagtctaggtttcatattcattgaattgctggggtttatgttattactcaactgaaataaaaagagaattgtcgttatccaggttagacaaaagcacgtgtagtttcaaacgcagtagccattattcccctctcctttacaggtgagattcaagggcagaaaggtaccatgcaagctgttatttaaatttacttattgattatgatcgacgactttgacagaaaaatgatgatgctttaattttgcgacctagactttctttttaacaagtataaaaaacatacctcaatttggtataaatcaataatttcgtcaatcttaaaagcatttttttacttgattcactaaatttaaatatatcttcgatatgccatatttcaaactcttgtcagaacatatgactctaaataaaaggtggagaagggttgaaaactttactaaatttaacaagatcttaggggtgtatttgactcataatgcaagattgaagtgtatttaagttcagttagtcaagtaaaggggtatttttaagattgtcaatatataattcggagatgaaactaataattcacactgaatttagggggtgttttcgatacttttcttttgtttcatttgttgtgtttaaaattaagttgatatgaaacaacaaataaattgtgatcccaagttttatatcagcaaaataaaatcaagcattgtatatctctcttttatttttctccttggtgtccgcattggagcccaactatattcgaatcgcactataggcttcatttttttcatactcggggcttaaatctgaaacctctggataagggtagaatatcttatatctctttttttttttttttttttttgaattgtctatccaacgtttggtatccacattgaagttcagttgattcaaatttgtgccgcataggctaagcggttcctaccaatgatctttccatatccaggaactcaaacgcaaaacgtttatttaagaaataaatagttccatccgctataccacatcacttgatagtgtggtatatctcattaagataccacattatatctatttaagggggtagaggggagggatgggtctagctaggtttgtggaaaggaggttagagagagagtggtttttcagtgtgtgtgtcaggtgatatcacgtgaaggggcacaataacaagttgttcatatccattcacttccaagaataccgctacgtaccactagctgcttcctccctatccccatctttgtcaaatttcctttccttttccaattactttttctttaatagacatggacgcaatttttccttcctttccccttcccctatattcttcttccttaatctctctttcccaataatagcaccatgcctgtagcttgatgcttagacgggtgcacgcgcactctcactcacccatatatatattcatagttagcagaagtacttatcatatataccaaaaaccacacaaatacattgtatcaagtgctgtcatactcaagcaaaaaaaaagaaaagaacaagatatagtactactgttttcatcaccattttggtcaatcatgatgattctgaacaagatatagtactagctagctaggtagaaaataaatctactaactttaattttcttcttattgcagctagcttgcttaattagtagcaaaatcaaaagaggttttagctgtgtgtttataccgtctttctcaagatctagacccaccacttagaccatctcaagctacagctactgctcaa

SEQ ID NO: 116 - DNA, 3000 bp, unidentified
  source 1..3000: mol_type = other DNA; note = the promoter regions of IT1 nucleotide sequences
aatttaaatattaatgttgtgaattctaaatactccacctgttttaaaaaatggtctagtttgactaggaacggagtttaagaaaaaaaaaaaattctaatcttgtggttccaaattaaagttatgtcaaatgtatcaaaatgccctttaatcttgtggtcctaaatatgtcacgtgaaaaattgaaactaaagtgttgccaaaaaaagaaagggatcattctttttgaaacaaacaaaaaagaaaactaagtcattctttttgaaacgaaaaaaatatactatatttatatattaggttctcgcaaacctgcaaataagggcggatgcacaacagaagttacgagtttgattaaatctactaattttggtttaaactttgtatatatatgtgtagaggagtcataaagttcaaatactgaatttactaattttatcaaagttgtgatgtgatctactgctaattaaagttgggtgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggtgcggagctagatatgcaaagggtttatataaatttatgtgatacatttttttagttaatctaaaaaagaataaaagcttctttttttaccaagatcgatttgaaatcacacaaatattcgtgaatttagatcataaatttcaaaaaaattttaataaaaggctatcttctgttctttgccttttagaggttaggtacatgctccatcccctcctttcttgttgttactatttgccatggcaggtagggatcaaggtcaaaccccaatgtatcacaggtcagtatagtccgaccgtgacaattttctaggggggattgtgtagaatcaaatatctattcataaattgagacattgagacgaatattcttctttacattattgagacgaaaaaaatattaatttttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaaccttcttttacttgacttactaaatttagatatatcttcgatctgccacataacataacaaatggtttcaaacggaacatatgactcttaataaaaggtggagaagggttgcaaactttactaaatttaacaagatcttaggtgtgtatttgactcatgttgcaagattgaagtgtatttaagttcagttaatcaagtaaagagatatttttaaggttgtcaatatataattcggagataaaactaataattcacattgaatttagagggtgtttttgacatttttcttttgtttcatttgttgagtttaaaattaagttgatacgaaacaacaaataaattgtgatcccaagttttatatgagcaaaataaaatcaagcattgtatatctctcttttatttttctccctggtatccgcattggagctcgactatattcgaatcgcactataggcttcatttttttcatactcgaggcttaaatctgaaacctctgggtaagggtagaatatcttatatctctttctttttttttggattttccattcaacgtttggtacccacattgaagttcagttaattcaaatttgcaccgcataggctaagcagttcctaccaatgttcttttcatacccaatgactcaaacacaagacgtttaattaagaaataaatagtctcatccgctataccacatcagttgatagtgtgatatatctcattaagataccacattatatctatttaaggggtaggggggatgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcatttcttccttcctttccccttcccctatattcttcctttatctctctttcacaataatagcaccatgcctgtagcttgatgcttagacgggtgcacacgcactctctcactcacacagctagaatatatatacatatatatatatatatatatatatatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatgtagtactagctaggtagaaaataaatctaccaactttaattttcttcttattgcagctagcttgcttaattagtagcaaaatcaaaagaggttttagctgtgtgtttatactgtctttctcaagatctagacccaccacttagaccatctcaagctacagctactgcgcaa

SEQ ID NO: 117 - DNA, 3000 bp, unidentified
  source 1..3000: mol_type = other DNA; note = the promoter regions of IT1 nucleotide sequences
gatccagagaatttaaatattaatgttgtgaattctaatactcccttcgtttaaaaaaaatggtctagtttaacttggaacagagtttaacaaaagaaataagactttttaattttgtggttctaaattaaagttatgtcaaatgtaccaaaatgccctttaatctagtggtcctaaacatgtcacgtgaaaagttaaagctaaagtgttggcaaaaaaagaaaggggtcattcttttttaaacaaacaaaaaaggaaattaagtcattttttttaaacgaatggagtatactatatttatatatttggttctcgcaatcgtgcaaataagggcggctgcacaatagaagttacgagtttgattaaatctactaattttagtttaaactttgtatatatgtgtagagaagtcataaagttcaaatagtgaatttacaaattttatcaaagttgtgatctactgctaattaaagttgggtgggcaggtgaggatggccggtattggggaatattgtatactgtacatctaaaagcaatttaggtaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagacctcatcactccctagtgagggcgcggagctagatatgcaaagggtttatataaatttatacgatacattttttttagttaatctaaaaaagaataaaagttcttttttttttttaccaagatcaatttgaaatcacacaaatatttgtgaatttagatcataaatttcaaaagtcttttaataaaagacaagcttatgttcttagccttttagaggttaggtacatgctccatcccctccttccttgttgttactatttgccatggccggtagggatcaaggtcaaaccccaatgcatcacaggtcagtatagcccgatcggtgacaattttctaaggggagattgtgtagaatcaaatatctattcataaattgagacgaatattcttctttacattattgagacgaagaaaatattaaattttcttaataaaaatcttagctcctcactatttagtctaggtttcatattcattgaattgctgggtttatgttactactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactgtgacagaaaaatgatgatgctttaattttgcgacctagcctttctttttaagaagtataaaaaacatacctcaatttggtataaatcaatagtttcgtcaatctttaaagcatttttttacttgactcactaaatatatctttgatctgtcacataacataataaatgatttcaaactcttgtcgtaacatatgactcttaataaaaagtggagaagggttgaaaactttactaaatctaacaagatcttagggggtgcatttgactcatattgcaagattgaagtgtattaaagttcagttagtcaagtaaaggggtatttttaagattatcaacatataattcgaagatgaaactaataattcacactgaatttagggggtgttttcgatacttttcttttgattcatttgttgagtttaaaattaagttagatatgaaacaacaaataaattgtgatcccaagttttatatcagcaaaataaattcaagcattgtatatctctcttttattttctctccggtgtccgtattggagcccgactatattcgaatcgcactataggcttcatttttttcatacttaggacttaaatctgaaacctctgggataatggtagaatatcttatatctctttctttttttttttttttgaattttccatccaacatttgatacccaccttgaagttcagttaattcaaatttgtgccgcataggctaagcggttcctaccaatgatctttccatacccacggactcaaacgtcttttaagacataaatagtctcatccattacaccatatcacttgatagtgtggtatatctcattaagattccacattatatctatttaaggggggtaggggggatggatctagctagggttgtggaaaggaggttagagagagagaggtttttcagtgtgtgtgtcaggtgatatcacgtgaagggccacgataacaagttgttcgtatccattcacttccaagaataccgctacgtaccactagctatttccttcctatccctccccatcttagtcaaatttcctttcctttccttttccaattactttttctttaatagacatggacgcatttcttccttcctttccccttcccctatattcttcttcctttatctctctttcacaataatagcaccatgcctgtagcttgacgggtgcacgcgcactctcactcacacagctagaatatatatacatatatatatatattcatagttagcagaagtagttatcatataccaaaaaccacacaaatacattgtatcaagtgctgtcatactcaagcaaaagaaagaaaagaacaagatatagtacaactgttttcatcaccattttggtcaatcatgatgattctgaacaagatatagtgtactagctaggtagtaaataaatctactaactttaattttcttcttattgcagctagcttgctcgcttaattagcagcaaaatcaaaagaggttttagctgtgtttatactgtatttttctcaagatctagacccaccacttagaccatctcaagctacagctacagctactgcgcaa

SEQ ID NO: 118 - DNA, 3000 bp, unidentified
  source 1..3000: mol_type = other DNA; note = the promoter regions of IT1 nucleotide sequences
cctccgtcccattttatgtggcaccattttctttttggtcagtcccaaaaagaatgtcacatttccttatatgataagtatttaaaggtacaattcctcttttatccctgttggtctcacttaatattaaagatagtactccaatatatttatgaggagagagaaaaatgaatttactttttaaaaggcaatttgataaacatttcaaagtctttatttatttattaaattccgtacccgatcaaatattgccacataaaatgggatggagggagtactgaatttactaattttatcaaagttgtgatctactgctaattaaagttgggtgggcaggtgaggatgcctggtattggagaatatatagtatactgtacatctaaaagcaattgaggcaaaaccatctctgtcactgatctctcacttcacaatactaacatatagagttggggccaaactgccaaagtagtcatgtagttgatgggaagagatagtacggatgaatggagaaaaaggtaggtgggtgagttgggggaagagatagggttctctttttagtgaaagtgacatgctctaaggtcaaaagttgcattcttctcaaagggcaattcagaccttcatcactccctagtgagggcgcggagctagatatgcaaagggtttatataaatttatgtgatacattttttttagttaattttaaaaaaaataaaagtttttttttttttttaccaagattgatttgaaatcacacaaatatttataaatttagatcataaatttcaaaagtcttttattaataaaaggctagcttctgttcttggccttttagaggttaggtacatgctccatcccctctttccttgttgttactatttgccatggccggtagggatcaaggtcaaatcccaatgaatcacatgtcagtatagtccgaccggtgacagttttctaagtgggattgtgtagaatcaaacgctaccaagaaaagtgtgaattacatgagaatttttctaggaattagtatgaaaatttgcaaaaaaataagtttcatacagatttttatactaatcccaagaaaattttcatgtaattcacacttttttttatagtgaaatatctattcataaattgagacattgagacgaatattcttctttacattattgagacgaagaaaatattaatttttttttaataagaatcttagctcctcactatttagtctaggtttcatattcattgaattgctggggtttatgctattactcaactgaaataaaaagagaattgtcgttatccaggttagacaaaagcacgtgtagtttcaaaacgcagtagccattattcccctctcctttacaggtgagattcaagggcagaaaggtaccatgcaagctgttatttaaatttacttattgattatgatcgacgactttgacagaaaaatgatgatgctttaattttgcgacctagcctttctttttaacaagtataaaaaacatacctcaatttggtataaatcaatagtttcatcaatcttaaaaatatttttttacttgactcactaaatttaaatatatttttgatatgccacatttcaaactcttgtcggaacatatgacttttaataaaaggtggagaagggttgaaaactttactaaatttaacaagatcttaggggtgtatttgactcataatgcaagattgaagtgtatttaagttcagttaatcaagtaaaggggtattcttaagattgtcattatataattcggagatgaaactaataattcacactgaatttagggggtgttttcaatacttttcttttgtttcatttgttgagtttaaaattaagttgatatgtaaacaaataaattgtgatcccaagttttatatcagcaaaataaaatcaagcattgtatatctctcttttatttttctccctggtgtccgcattggagtccgactatattcgaatctcactataggcatcatttttttcatactcagggtttaaatctaaaatctctggataagggtagaatattttatctctctttcttttttcttttttttttttgaattttgcatccaacgtttggtatccacattgaaattcagttgattcaaatttgcgtaacataggctaagcggttcctaccaatgatcttttcatacccagaaattcaaacgcaagacgtttgtttaaaaaataaatagtaccatccgctataccacatcacttggtagtgtgtatatctaattaagataccacattatatctatttaagggggtaaggggggatgggtctagctagagttgtggaaaggaggttagagagagagagtggtttttcagtgtgtgtgtcaggtgatatcacgtgaaggggcacggtaacaagttgttcatatccattcacttccaagaataccgctacgtaccactagctgcttccttcctatccccatctttgtcaaatttcctttccttttccaattactttttctttaatagacatggacgcaattcttccttcctttccccttcccctatattcttcttcctttatctctctttcacaataatagcaccatgcctgtagcttgatgctttgacgggtacacacactctcactcacacagctagaatatatatatatatattcatagttagcagaagtacttatcatatataccaaaaaccacacaaatacattgtatcaagtgctgtcatactcaagcaaaaaaaaagaaaagaacaagatatagtactactgttttcatcaccatttgggtcaatcatgataattctgaacaagatatagtactagctagctaggtagaaaataaatatacatttgtttaattttcttcttattgcagcttagcttgcttaattagcagcaaaatcaaaagaggttttagctgtgtttataccgtctttctcaagatctagacccaccacttagaccatctcaagctacagctactgcgcaa

SEQ ID NO: 119 - DNA, 3000 bp, unidentified
  source 1..3000: mol_type = other DNA; note = the promoter regions of IT1 nucleotide sequences
agaatttaaatattaatgttgtgaattctaatactccacttgttttaaaaaatggtctagtttgacttgaaacggagtttaagaaaagaaagaaaactttctaatcttgtggttccaaattaaagttatgtcaaatgtaccaaaatatcctttaattttgtagtcttaaatatgtcacgtgaaaaattgaagttaaagtgttgtcaaaaaaggaaaggaaccattctttttgaaacaaactaaaaaaaaaactagatcatttttttttaaacggagaaagtatactatatttatatatttggttctcgcaaacctgcaaataagggcggatgcacaacagaagttacgagtttgattaaatctactaattttagtttaaactttgtatatatgtgtagaggagtcataaagttcaaatactgaatttactgattttatcaaagttgtgatctactgctaattaaagttgggtgggcaggtggggatgcccggtattggggaatgtagtatactgtacatctaaaagcaattttggcaaaaccatctctgtcactgatctctcacttcacaatactaacatatagagttggggccaaagaagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagaccttcatcactccctagtgagggcgcggagctagatatgcaacgagtttatataaatttatgtgatatattttttttaattaatctaaaaaagaataaaaggtttttttttttaccaagatcgatttgaaatcacacaaatatttgtgaatttagatcataaatttcaagtcttttaataaaaagctaggttctgttcttggccttttagaggttaggtacatgctccatccactccttccttgttgttattatttgccatgactggtagggatcaaggtcaaaccccaatgcatcataggtcagtatagtccgaccgtgacgattttctaggggggattgtgtagaataaaatatctattcataaattgagacattgagacgaatattcttcttcacattattgagacaaagaaaatattaaattttcttaataaaaatcttagctcctcactatttagtctaggtttcatattcaatgaattgctgtggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttgcgacctagccattctttttaagaagaataaaaaacatacctcgatttggtataaatcaatagtttcgtcagtcttaaaagcattcttttacttgagtcactaaatttagatatttctttgatctgccacataacataacaaatgatttcaaactcttgttggaacatatgactcttgataaaaggtggagaagggttgaaaactttactaaatttaacaagatcttaggtgtgtatttgactcatgttgcaagattgaagtgtatttaagtttagttagtcaagtaaagaggtatttttaagattgttaatatataattcggagataaaactaataattcacactgaatttagggggtgttttcaatacttttcttttgtttcatttcttgagtttaaaattaaggtgatatgaaacaacaaataaattgtgatcccaagttttaatgaacaaaataaaatcaagcattgtatatctctcttttatttttctccctggtgtccgcattggagctcaactatattcgaatcacactataggcttcatttttttcatactcaaggcttaaatctgaaacctgtgggtaagggtagaatatcttatatctctttttatttttttttggattttccatccaacgtttgttatccacattgaagttcagttaattcaactttgcgccgcataggctaagtggttcctaccaatgatctttccataccctgggattcaaatacaaaacatttatttaagaaataaatagtctcatccgctataccatatcagttgatagtgtggtatatctcattaagataccacattatatctctttaagggggtaggggggatgggtctagctagggttgtggaaatgaggttagagagagggtggtttttcagtgtgtgtgtcaggtgaaatcacgtgaaggggcacgataacaagttgttcatatccattcacttccaagaataccgctacgtacagtcactactgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctcactcacacagctagaatatatatatctatatatatatatatatttatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaatcaatctactaactttaattttcttattgcagctagcttgcttgcttaattagcagcaaaatcaaaagaggttttagctgtgtttatactgtctttctcaagatctagacccaccacttagaccatctcaagctacagctactgcgcaa

SEQ ID NO: 120 - DNA, 3000 bp, unidentified
  source 1..3000: mol_type = other DNA; note = the promoter regions of IT1 nucleotide sequences
gtagtgcgtgctgttaggttgccgatccagagaatttaaatattaatgttgtgaattctaatactctctccgttaaaaaaaaaatgatctagtttgacttggaacgaaatttaacaaaagaaaaaagactttaattttgtggttttaaattaaagttatgtcaaatgtaccaaaatattctttaatcttgtggttctaaacatgtcacgtgaaaagttgaagctaaagtgttgccaaaaaaagaaaaggatcattctttttgaaacaaacaaaaaaggaaattaagtcattctttttaaaacggggggagtatactatatttatatatttggttctcgcaaacctgcaaataagggcggatgcacaatagaaattgcgagtttgattaaatctactaattttagtttaaactttgtatatatgcgtagagaagtcataaagtgcaaatactgagtttactaattttatcaaagttgtgatctactgctaattaaagttgggtgggcaggtggggatgccccgtattggggaatgtagtatactgtacatctaaaagcaattttggcaaaaccatctctgtcactgatctctcacttcacaatactaacatatagagttggggccaaagtagtcatgtagctgatgggaagagatagtacggaggaatggagaaaaaaggtaggtgggtgagttgggggaagagatagggttctctttttagtgaaagtgacatgctctaaggtcaaaagttgcattcttctcaaagggcaattcagaccttcatcactccctagtgagggggcggagctagatatgcaaagggtttatataaatttatgtgatacattgtttttagttaatttaaaaaagaataaaagttttttttttaccaagatcgatttgaaatcacacaaatatttgtgaatttagatcataaatttcaaaagtcttttaataaaatgctagcttctgttcttggccttttagaggttaggtacatgctccatcccctcctttcttgttgttactatttgctatggccggtagggatcaaggtcaaatcccaatgtatcacaggtcagtatagtccgaccgtgacaattttctaggggggattgtgtagaatcaaatatctattcataaattgagacattgagacgaatattcttctttacattattgagacgaaaaaaatattaaattttcttaataaaaatcttagctcctcactatttagtctaggtttcatattcaatgaattgctggggttatgttgttactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatagacgactttgacagaaaaatgatgatgctttaattttgcgacctagccattctttttaagaagaataaaaaacatacctcaatttggtataaatcaatagttttgtcaattttaaaaacattcttttacttgactcactaaatttagatatatcttcgatctgccacataatataacaaataatttcaaactcttgtcggaacatatgacttttaataaaaggtggagaagggttgaaaactttactaaatttaacaagatcttaggtgtgtatttgactcatgttgcaagattgaagtgtatttaagttcagttagttaagtaaagatgtatttttaaggttgtcaatatataattcggagataaaactaataattcacactgaatttagggggtattttcgatacttttcttttgtttcatttgttgagtttaaaattaagttgatatgaaacaacaaataaattgtgatcccaagttttatatcagcaaaataaaatcagtcattgtatatctctcttttatttttctccttggtgtccgcattggagcccgactgtattcgaatcgtactataggcttcattttttgcatactcagggcttaaatttgaaacctctgggtaagggtagaatatcctatatctctttctttcttctttttttttttgaattttccatccaacgtttggtacccacattgaagtttagttaattcaaatttgcgccgcataggctaagcggctcctaccaatgatctttccatacccagggactcaaacgcaaaacatttatttaagaaataaataatcccatccgctgtaccacatcagttgatagtgtggtatatctcattaagataccacattatatctatttaagggggtaggggggatgggtctagctagggttgtggaaatgaggttagagagagggtggtttttcagtgtgtgtgtcaggtgaaatcacgtgaaggggcacgacaacaagttgttcatatccattcacttccaagaataccgctacgtaccactagctgctttcttcttatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctcactcacacagctagaatatatatattcatagttagcagaagtacttatcatataccaaaaaccacacaaatacattgtatcaagtgctgtcatactcaagcaaaagaaaaagatatagtactactgttttcatcaccattttggtcaatcatgatgattctgaacaaagatatagtactagctaggtagaaaataaatctactaactttaattttcttcttattgcagctagcttgcttaattagtagcaaaatcaaaagaggttttagctgtgtttatactgtctttctcaagatctagacccaccacttagaccatctcaagctacagctactgggcaa

SEQ ID NO: 121 - DNA, 3000 bp, unidentified
  source 1..3000: mol_type = other DNA; note = the promoter regions of IT1 nucleotide sequences
ccagagaatttaaatattaatgttgtgaattctaatactccacctgttttaaaaaatggtctagtttgatttggaacggagtttaagaatagaaagaagactttctaatcttgtggttccaaattaaagttatgtcaaatgtaccaaaataccctttaatcttgtggtcttaaacatgccacgtgaaaaattgaagttaaagtgttgccaaaaaaggaaaggagccattctttttgaaacaaactaaaaaagaaactagatcatatttttttaaacggagaaagtatactgtatttatatatttggttcttgcagacctgcaaataagggcggatgcacaacagaagttacgagtttgattaaatctactaattttagtttaaactttgtatatatgtgtagaggagtcataaagtgaatttactaattttattaaagttgtgatctactgctaattaaagttgggtgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagaccttcatcactctctagtgagggtgcggagctagatatgcaaggagtttatataaatttatgtgatatattttttttaattaatctaaaaaagaataaaagttttttttttttaccaagatcgatttgaaatcacacaaatatttgtgaatttagatcataaatttcaaaagtcttttaataaaaagctaggttctgttcttggccttttagaggttaggtacatgctccatccactccttccttgttgttattatttaccatgactggtagggatcaaggtcaaaccccaatgcatcacaggtcagtatagtccgaccgtaacgattttctaggggggattgtgtagaatcaaatatctattcataaattgagacattaagacgaatattcttcttcacattattgagacaaagaaaatattaaattttcttaataaaaatcttagctcctcactatttagtctaggtttcatattcaatgaattgctgtggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttgcgacctagccattctttttaagaagaataaaaaacatacctcgatttggtataaatcaatagtttcgtcaatcttaaaagcattctttaacttgagtcactaaatttagatatttctttaatctgccacataacataataaatgatttcaaactcttgtcggaacatatgattcttaataaaaggtggagaagggttgaaaactttactaaatttaacaagatcttaggtgtgtatttgactcatgttgcaagattgaagtgtatttaagtttaattagtcaagtaaagaggcatttttaaggttgttaatatataattcggagataaaactaataattcacactgaatttaaggagtgttttcaatacttttcctttgtttcatttcttgagttcaaaattaagttgatatgaagcaacaaataaattgtgatcccaagttttatatgagcaaaataaaatcaagcattgtgtatctctcttttatttttctccctggtgtctgcattggagctcaactatattcgaatcgcactataggcttcatttttttcatactctggggcttaaatcttaaacctgtgggtaagggtagaatatctaatatctctttctcttttttttttttggatttttcatccaatgtttgttacccacattgaagtttagttaattcaaatttgcgccgcataggctaagtggttcctaccaatgatcttcccatacccagggactcaaacgcaaaacatttatttaaaaaataaatagtctcatccgctataccacgtcagttgatgtggtatatctcattaagataccacattatatctatttaagggggtaggggggatgggtctaactagggttgtggaaatgaggttagagagagggtggtttttcagtgtgtgtgtcaggtgaaatcacgtgaaggggcacgataacaagttgttcatatccattcacttccaagaataccgctacgtacagtcactagctgctttcttcttatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctcactcacacagctagaatatatatatataatatatatatattcatagttagcagaaatacttatcatataccaaaaaccacacaaatacattgtatcaagtgctgtcatactcgagcaaaagaaagaaaagaacaagatatagtactactgttttcatcaccattttggtcaatcatgatgattctgaacaaagatatagtactagctaggtagaaaataaatctactaactttaattttcttcttattgcagctagcttgcttgcttaattagcagcaaaatcaaaagaggttttagctgtgtttatactgtctttctcaagatctagacccaccacttagaccatctcaagctacagctactgctactgcgcaa

SEQ ID NO: 122 - DNA, 3000 bp, unidentified
  source 1..3000: mol_type = other DNA; note = the promoter regions of IT1 nucleotide sequences
gttgccgatccagagaatttaaatattaatgttgtgaattctaatactccacctgttttaaaaaatggtctagtttgacttggaatagagttaaagaaaagaaagaagactttctaattttgtggtttctaattaaagttatgtcaaatgtaccaaaataccctttaattttgtggtcttaaacatgtcacgtgacaaattaaagttaaaatgttgccaaaaaaaaaaaaaggagccattccttttgaaacaaactaaaaaagaaactaggtcatttttttttaaacagagaaagtatactatatttatatatttgattctcgcaaacctgcaaataagggcggatgcacaacagaagttacgagtttgattaaatctactaattttagtttaaactttgtatatttgtgtagaggagtcataaagttcaaatactgaatttactaattttatcaaagttgtaaatctactgctaattaaagttgggtgggcaggtggggatgccctgtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagaccttcatcactccctagtgagggcgcggagctagatatgcaaggagtttatataaattcatgtgatatattttttttaattaatctaaaagagaataaaagttttttttttttaccaagatcgatttgaaatcacacaaatatttgtgaatttagatcataaatttcaaaagtcttttaataaaaagctaggttctgttcttggccttttagaggttaggtacatgctccatccactccttccttgttgttattatttgccatgactggtagggatcaaggtcaaacccaccaatgcatcacaggtcaatatagtccaaccgtgacgattttctaggggggattgtgtagaataaaatatctattcataaattgagacattgagacgaatattcttcttcacattattgagacaaagaaaatattaaattttcttaataaaaatcttagctcctcactatttagtctaggtttcatattcaatgaattgctgtggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttgcgacctagcctttctttttaagaagaataaaaaacatacctcgatttggtataaatcaatagtttcgtcaatcttataaacattcttttacttgagtcactaagtttagatatttctttgagctgccacataatataacaaatgattttaaactcttgtcggaacatatgactcttaataaaaggtggagaagtgttgaaaactttactaaatttaacaagatcttaggtgtgtatttgactcatgttgcaagattgaagtgtatttaagtttagttagtcaagtaaaaaggtatttttaaggttgtcaatatataatttggagataaaactaataattcacactgaatttagggggtgttttcaatacttttcttttgtttcatttcttgagtttaaaattaagttgatatgaaacaacaaataaattgtgatcccaagttttatatgagcaaaataaaatcaagcattgtatatctctcttttatttttttccttggtgtccgcattggagctcgactatattcgaatcgcactataggcttcatttttttcatactcagggcttaaatcttaaacctgtgggcaagggtagaatatcttatatctttttttttatttttggatttttcatccaacgtttgttacccacattgaagttcagttaattcaaatttgcgccgcataggctaagtggttcctaccaatgatcttcccatacccagggactcaaacgcaaaacatttatttaagaaataaatagtctcatccatatctcattaagataccacattatatctatttaagggggtagtggggatgggtctagctagggttgtggaaatgaggttagagagagggtggtttttcagtgtgtgtgtcaggtgaaatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctcactctcactcacacagctagaatatatatatatatatatatatatatatataatatattcatagttagcagaaatactcatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtaaaaaataaatctactaactttttcttcttattgcagctagcttgcttgcttaattagcagcaaaatcaaaagaggttttagctgtgtttacacggtctttctcaagatctagacccaccacttagaccatctcaagctacagctactgcgcaa

SEQ ID NO: 123 - DNA, 3000 bp, unidentified
  source 1..3000: mol_type = other DNA; note = the promoter regions of IT1 nucleotide sequences
agcacaaattttttaggttgctgatccagagaatttaaatattaatgttgtgaattctaatactccacctgttttaaaaaatggtctagtttgacttgaaacgaattttaagaaaagaaagaagactttctaatcttatggtttcaaattaaagttatgtcaaatgtaccaaaatatcctttaatcttgtgattttaaacttgtcacgtgaaaaattaaagttaaagtattgccaaaaaaagaaaggagtcattctttttgaaacaaactaaaaaagaaactagatattttttttaaacggagaaagtatactatatttatatatttggttctcgcaaacctgcaaataagggcggatgcacaacagaagttacgagtttgattaaatctactaattttagtttaaactttgtatatatgtagaggagtcataaagttcaaatactgaatttactaattttatcaaagttgtgatctactgctaattaaagttgggtgggcaggtggggatgcccggtattggggaatgtagtatactgtacatctaaaagcaattttggcaaaaccatatctgtcactgatctctcacttcacaatactaacatatagagctggggccaaagaagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagaccttcatcactcccctagatatgcaaggagtttatataaatttatgtgatatttttttttttattaatctaaaaaaaaatataagttttttttttttaccaagatcgatttgaaatcacacaaatatttgtgaatttagatcataaatttcaaaagtcttttaataaaaagctacgttctgttcttggccttttagaggttaggtacatgctccatccactccttccttcttgttattatttgccatgactggtagagatcaaggtcaaaccccaatgcatcatgggtcagtatagtccgaccgtaacgattttctagggggggttgtgtagaatcaaatatatattcataaattgagacattgagacgaatattcttctttacattattgagatgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatggacgactttgacagaaaaatgatgatgctttaattttacgacctagcctttctttttaagaagaataaaaaacatacctcgatttggtataatcaatagtttcgtcaatcttaaaaacattcttttacttgaattactaaatttagatatttctttgatctgtcacataacataacaaataatttcaaactcttgtcggaacatatgactcttaataaaaggcggagaagggttgaaaattttactaaatttaacaagatcttaggtgtgtatttgactcatgttgcaagattgaagtgtatttaagtttaattaatcaagtaaagaggtatttttaagattgtcaatatataattcgaagataaaactaataattcacattgaatttaggaggtgttttcaatacttttcttttgtttcatttcttgagtttaaaattaagttgatatgaaacaacaaataaattgtgatcccaagttttatatgagcaaaataaaatcaagcattgtatatctctcttttatttttctccctggtgtccgcattggagtccgactttattcgaatcgcactataggcttcatttttttcatactcagggcttaaatctgaaactagtgggtaagggtagaatatcttatatcttttttttttttttttttttttggattttccatccaatgtttgttacccacattaaagttcagttaattcaaatttgcgccgcataggctaagtggttcctaccaatgatctttccatacccaaggactcaaacgcaaaatatttatttaagaaataaatagtctcatccgctataccacatcagttgatagtgtggtatatctcattaagataccacattatatttaagggggtaggggggatgggtctagctagggttgtggaaatgaggttagagagagggtggtttttcagtgtgtgtgtcaggtgaaatcacgtgaaggggcacgacaagttgttcatatccattcacttccaagaataccgctacgtacagtcactggctgctttcttcctatccccatctttgtcaaatttcctttccttttccaattattactttttctttagtagacatggacgcaattcttccttcctttccccttcccctatattcttcctttatctctcttacacaataatagcaccatgcctgtagcttgatgcttagacgggtgcacacgcactctcactcacacagctagaatatatatatatatatatatatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctactaactttaattttcgtcttattgcagctagcttgcttaattagcagcaaaatcaaaagaggttttagctgtgtttatactgtctttctcaagatctagacccaccacttagaccatctcaagctacagctacagctactgcgcaa

SEQ ID NO: 124 - DNA, 3000 bp, unidentified
  source 1..3000: mol_type = other DNA; note = the promoter regions of IT1 nucleotide sequences
atgaaaaaattaggaagatttccattatctaagtaagaatctgtttttttatcatgcatttttccagtaggctgattcagtgggaaaatcatgttttatagcacaatttttttaggttgccgatccagagaatttaaatattaatgttgtgaattctaatactccacctgttttaaaaaatggtctagtttgacttggaacggagtttaagatttttttttttttctaatcttgtggttccaaattaatattatgtcaaatataccaaaatatcctttaattttgaaacggagaaagtatactatatttatatatttggttctcgcaaacctgcaaataagggaggatgcacaacagaagttacgagtttgattaaatctactaattttagtttaaactttgtgtagaggagtcataaagttcaaatactgaatttactaattttataaaagttgtgatctactgctaattaaagttgggtgggcaggtggggatgcccagtattggggaatgtagtatactgtacatctaaaagcaattttggcaaaaccatctctgtcactgatctctcacttcacaatactaacatatagagttggggccaaagcagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggcgcggagctagatatgcaaagggtttatataaatttatgtgatacattttttttagttaatctaaaaaagaataaaagtttttttttttttttaccaagatcgatctgaaatcacataaatatttgtgaatttagatcataaatttcaaaagtcttttaataaaaggctagcttctgttcttcgccttttagaggttaggtacatgctccatcccctcctttctcgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaatattcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcaatttggtataaaccaatagtttcgtcaatctaaaaaatattattttacttgactcactaaatttagatatatcttcgatctaccacataacataacaaatggtttcaaacttttgtcggaacatatgactcttaataaaaggtggagaagggttgaaaactttactaaatttaacaagatcctaggtgtgtatttgactcatgttgcaagattgaagtgtatttaagtttagttagtcaagcaaagagatatttttaaggttgtcaatatataattcagagataaaactaataattcacactgaatttaggaggtgttttcaatacttttcttttgtttcatttcttgagtttaaaattaagttgatatgaaacaacaaataaattgtgatcccaagttttatatgagcaaaataaaatcaagcattgtatatctctcttttatttttctcccttgtgtccgcattggagcccgactatattcgaatcgcactataggcttcatttttttcatactcggggcttaaatctgaaacctatgggtaagggtagaatatcttatatctctttttttttttttggattttccatccaatgtttgttacctacattgaagttcagttaattcaaatttgtgccgcataggctaagcggttcctaccaatgatctttccatacccagggactcaaacgcaaaacatttatttaagaaataaatagtctcatccgctataccacatcagttgatagtgtggtatatctcattatgataccacattatatctatttaaggggggtaggggggatgggtctagctagggttgtggaaatgaggttagagagaaggtggtatttcagtgtgtgtgtcaggtgaaatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctctcactcacacagctagaatatatatatatatatatattcatagttaacagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctactaactttaattttcttcttattgcagctagcttgcttaattagtagcaaaatcaaaagaggttttagctgtgtttatactgtctttctcaagatctagacccaccacttagaccatctcaagctacagctactgcgcaa

SEQ ID NO: 125 - DNA, 3000 bp, unidentified
  source 1..3000: mol_type = other DNA; note = the promoter regions of IT1 nucleotide sequences
tttttgaaacggaggaatcatactatatttatatatttggttctcgcaaacctgcaaataagggcggatgcacaacaaaagttacgagtttgattaaatctactaattttagtttgaactttgtatactccctccgtttcacaaagaatgacctggtttgacttgacacggagtttaagtaaataaagaagacttttgaatcttgtggtcctaaattaatgttatgtcatatgtacaaaattgcccttaatcttgtggtcttaaacatgccacgtggaaagcaaaaagtaaaatgttaccaaaaaagaaaagaggtcattctttttgaaactgactaaaaagaaaaagaagtcatttttttgtgaaacggagagagtatatgtatagaggagtcataaagttcaaatactaaatttactaattttatcaaagttatgatctactgctaattaaagttgggtgggcaggtggggatgcccagtattggggaatgtagtatactgtacatctaaaagcaattttggcaaaaccatctctgtcactgatctctcacttcacaatactaacatatacagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagaccttcatcactccctagtgagggcgcggagctagatatgcaaagggtttatataaatttatgtgatatattgtttttagttaatctaaaaaagaataaaagttttttattttaccaagatcgatttgaaatcacacaaatatttgtgaatttagatcataaattttaattttattttaataaaaggttagcttctgttcttggccttttagaggttaggtacatgctccatccccttatttcttgttattactatttgccatggccggtagggatcaaggtcaaaccccaatgcatcacagcagtatagtctgaccgtgtcaattttctatggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagcaattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcaatttggtataaaccaatagtttcgtcaatctaaaaaaatattcttttacttgactcactaaatttagatatatctttgatctgccacataacataacaaattgtttcaaacttttgttggaacatatgactcttaataaaaggtggagaagggttgaaaactttactaaatttaacaagatcttaggtgtgtatttgactcatgttgcaagattgaagtgtatttaagtttagttagtcaagtaaagagatatttttaaggttgtcaatatataattcgaagataaaactaataattcacactgaatttagggagtgttttcaatacttttcttttgtttcatttcttgagtttaaaattaagttgatatgaaacaacaaataaattgtgatcccaagttttatatgagcaaaataaaatcaagcattgtatatctctcttttatttttctccctggtgtctgcattggagcccgactatattcgaatcgcactataggcttcatttttttcatactcagagcttaaatctgaaacctgtgggtaagggtaggatatcttatatctctttctttttttttctttttttttggattttccattcaacatttgttacccacattgaagttcaattaattcaaatttgctccgcataggctaagcagttcctaccaatgatctttccatacctaggaactcaaacgcaaaacatttatttaagaaataaatagtctcattcgctataccacatcagttgatagtgtgatatactatctcattaagataccacattatatctatttaagggggtaggggggacgggtctagctagggttgtggaaatgaggttagagagagggtggtttttcagtgtgtgtgtcaggtgaaatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctcactctcactcacacagctagaatatatatattcatatatatatatatatattcatagttagcagaagtacttatcatataccaaaaaccatacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctactaactttaattttcttcttattgcagctagcttgcttaattagtagcaaaatcaaaagaggttttagctgtgtttatactgtctttctcaagatctagacccaccacttagaccatctcaagctacagctactgcgcaa

SEQ ID NO: 126 - DNA, 3000 bp, unidentified
  source 1..3000: mol_type = other DNA; note = the promoter regions of IT1 nucleotide sequences
agaatatgttttttcatcatgcattttctcaataagctgattcggtatgttttatagcacaatttttttattgatacggatgggagaaacctttatttctagtagtgtgtgctgttaggttgccgatccagagaatttaactattaatgttgtgaattctaatactccctttgtttgaaaaagaatggtctagtttgatttggaacagattttaacaaaagaaataagactttttaatcttgtggttttaaactaaagttatgtcaaatgtatcaaaatgtcttttaatcttgtgattctaaacatgtcaggtgaaaagttaaagttaaagtattgctaaaaaaagaaaggggttattcttttttaaataagcaaaaaagaaattaaatcattcattttaaaacggaaacactatactatatttacatatttggttctcgcaatcctgcaaataagggcggatgcacaatagaggttaggagtttgattaaatctactaattttagtttaaactttgtatatatgtgtacagaagtcataaagttcaaatattgaacttactaattttatcacagttgtgatctactgctaattaaagttgggtgggcaggtgaggatgcccggaattggggaatattgtatactgtacatctaaaagcaatttaggcaaaaccatctctgtcactgacctctcacttcacaatactaacatacagagttggggccaaagtagtcatgtagttgatgggaagagatagtacggaggaatggagaaaaaggtaggtggatgagttgggggaagagatagggttctctttttagtgaaagtgacatgctctaaggtcaaaagttgcattcttctcaaagggcaattcagaccttcatcactccctagtaaaggcgcggaactagatatgcaaggggtttatataaatttatgtgatgtaatatttttagttaatctgaaaaagaataaaaattcttttttttttttaccaagatcaatttgaaatcacacaaatatttgtgaatttaaatcataaatttgaaaagtcatttaaaaaaaggccagcttctgttcttggaggttaggtacatgctccatcccctccttccttgttgttgctatttgcaatggccggtaggaatcaaggtcaaactccagcaatttctagagggggattgtgtagaaccaaatatctattcataaattgagacgaatattcttctttacattattgagacaaagatattattaaattttttcatattcattgaattgctggtatttatgttattactcaactgaaataaaaagagaattgtcgttatccaggttagacaaaagcacgtgtagtttcaaacgcagtagccattattctcctctcctttacaggtgagattcaggggcagaaaggtaccatgcaagctgttatttaaatttacttattgattatgatcgacgactttgacagaaaaatgatgatgctttaattttgcgacctagcctttctttttaagaagtataaaaaacatacctcaatttggtataaatcagtagtttcatcaatctgcattcttttacttgactcactagatatatctttaatctgtagcaaatgatttcaaattcttaccggaacatatgactcttaataaaaggtggagaagggttgaaactttattatatttaacagatcttatgggtgtatttgactcatgttgtaagattgaagtgtatttaagtttagttagtcaaataaagagatatttttaaggttgtcaacatataattagtagataaaattaataattcacactgaatttagggggtgttttcgatacttttcttttgtttcatttgttgagtttaaaattaagttgatatgaaaaacaaataaattgtgatcccaagttttatatcagcaaaaataaaatcaagcattgtatatctctcctttatttttctccttggtgtccgtattggaacccgattatattcgaatcgcactataggcttcatttttttcatactcagggcttacatctgaaacctctgggtaagggtagaatgtcttatatctctttcttttttcttttttttttcggattttccatccaacgtttgatacccaccttgaagttcagttaatttaaatttgcgccgcataggttaagcggttcctaccaataatcttttcataccaagagactcaaacattttttaagaaataaatagttacatccgctataccatatcacttgatagtgtggtatatctcattaagataacacattatatctatttaaggggtaaggggggatgggtctagctagggttgtggaaaggaggttagagagagagagagaggtttttcagtctgtgtcagttgatatcacgtgaaggggcacgattacaagttgttcgtatccattcacttccaagaataccgctacgtaccactagctacttccttcctatccccatctttgtcaaatttcctttccctttccaattactttttctttaatagacatggacgcaattcatccttcctttccccttcccctatattcttcttcctttatctctctttcacaataatagcacgcaccatgcctgtagcttgatgggtgcacgcgcactctcactcacacagctagaatatatatattcatagttagcagaagtacttatcatataccaaaaaccacacaaatacattgtatcaagtgctgtcatactcgagcaaaagaaagacaagaacaagatatagtacaactgtttgcataaaccattttggtcaatcatgatgattctgaacaagatatagtactagctaggtagaaaataaatctactaactttatttttcttcttattgcagctagcttgcttaattagcagcaaaatcaaaagaggttttagctgtgtttatactgtctttctcaagatctagacccaccacttagaccgtctcaagctacagctactgcgcaa

SEQ ID NO: 127 - DNA, 3000 bp, unidentified
  source 1..3000: mol_type = other DNA; note = the promoter regions of IT1 nucleotide sequences
gagtcataaagttcaaatactccctccgttccattttatgtggcaccatttcttttttggtcagtcctaaaaagaatgtcacatttccttatatgataagtatttaaaggtacaattcctcttttacctttgttggtcccacttaatattaaagatagcactccaatatatttacgaggagagaaaaatgagtctactttttaaagggcaatttgataaacatttcaaagtctttatttatttattaaactctatatccgatcaaatattgccacataaaattggacggagagagtactgaatttgctaattttatcaaagttgtgatctactgctaattaaagttgggtgggcaggtgaggatggctggtattggagaatatagtatactgtacatctaaaagcaattgaggcagaaccatctctgtcactgatctctcacttcacaatactaacatatagagttggggccaaagtagtcatgtagttgatgggaagagatagtacggatgaatggagaaaaaggtaggtgggtgagttgggggaagagatagggttctctttttagtgaaagtgacatgctctaaggtcaaaagttgcattcttctcaaagggcaattcggaccttcatcactccctagtgagggtgcggagttagatatgcaaagggtttatataaatttatgtgatacatttttttttagttaatttaaaaaagaatattattatttttaccaagattgatttgaaatcacacaaatatttataaatttagatcataaatttcaaaagtcttttaataaaagactagcttctgttcttggccttttagaggttaggtacatgctccatcccctccttccttgttgttactatttgccatggccggtagggatcaaggtcaaatcccaatgaatcacaggtcagtatagtccgaccggtgacagttttctaagtgggattgtgtagaatcaaacgctaccaagaaaagcgtgaattacatgagaattttttctagaaattagtatgaaaatttgcaaaaaaataagtttcctacagatttttatactaattcccaagaaaattttcatgtaattcactatttttttatagtgaaatatctattcataaattgagacgaatattcttctttacattattgagacgaagaaaatattaattttttttaataagaatcttagctcctcactattttgtcgaggtttcatattcattgaattgctggggtttatgttattactcaactgaaataaaaagagaattgtcgttatccaggttagacaaaagcacgtgtagtttcaaacgcagtagccattattcccctctcctttacaggtgagattcaagggcagaaaggtaccatgcaagctgttatttaaatttacttattgattatgatcgacgactttgacagaaaaatgatgatgctttaattttgcgacctagcctttctttttaagaagtataaaaaacatacctcaatttggtataaatcaataatttcgtcaaacttaaaatcatttttttaattgactcactaaatttaaatatatcttcgatatgtcacatttcaaattcttgtcggaacatatgactcttaataaaaggtggagaagggttgaaaactttactaaatttaacaagatcttaggggtgtatttgactcataatgcaagattgaagtgtatttaagttcagttagtcaagtaaaggggtatttttaaggttgtcaatatataattcggagatgaaactaataattcacactgaatttagggggtgttttcgatacttttcttttgtttcatttgttgagtttaaaattaagttgatataaacaacaaataaattgtgatcccaagttttatatcagcaaaataaaatcaagcattgtatatctctcttttatatttctctctggtgtccgcattggagcccgactatgttcaaatcgcagtataggcttcatttttttcatactcagggcttaaatctgaaacctctggataagggtagaatatcttatatctcttcttcttttttttggattttccatccaatgtttggtatccacattgaagtttagttgattcaaatttgcgccgcataggctaagcggttcctaccaataatctttccatacccagaaactcaaacgcaagacgtttgtttaagaaaaaaatagtttcatctgctataccacatcacttgatagtgtggtatatctaattaagataccacattatatctatttaagggggtagtgggggatgggtctagctagagttgtggaaaggaggttagagagagagtggtttttcagtgtgtgtgtcaggtgatatcacgtgaaggggcacgataacaagttgttcatatccattcacttccaagaataccgctacgtaccactagctgcttccttcctatccccatctttgtcaaatttcctttccttttccaattactttttctttaatagacatggacgcatttcttccttcctttccccttcccctatattcttcttcctttatctctctttcacaataatagcaccatgcctgtagcttgatgcttagacgggtgcacacacactctcactcacacagctagaatatatatatatatatatatatttatatatattcatagttagcagaagtacttatcatatataccaaaaaccacacaaatacattgtatcaagtgctgtcatactcaagcaaaaaaaaaagaaaagaacaagatatagtactactgttttcatcaccatttgggtcaatcatgataattctgaacaagatatagtactagctagctaggtagaaaataaatctacatttgtttaattttcttcttattgcagctaccttgcttaattagcagcaaaatcaaaagaggttttagctgtgtttataccgtctttctcaagatctagacccaccacttagaccatctcaagctacagctactgcgcaa

SEQ ID NO: 128 - DNA, 3000 bp, unidentified
  source 1..3000: mol_type = other DNA; note = the promoter regions of IT1 nucleotide sequences
ccagagaatttaaatattaatgttgtgaattctaatactccacctgttttaaaaaatggtctagtttggcttgaaacggagtttaagaaaagaaagaaagactttctaatcttgtggttccaaattaaagttatgtcaaatgtaccaaaataccctttaatcttgtggtcttaaacatgtcacgtgaaaaattgaagttaaagtgttgtcaaaaaaggaaaggagccattcttttttaaacaaactaaaaaaaaactaggttattttttttaaacggagaaagtatactatatttatatatttggttctcgcaaacctgcaaataagggcagatgcacaacagaagttacgagtttgattaaatctactaattttagtttaaactttgtatatatgtgtagaggagtcataaagttcaaatactgaatttactgattttatcaaagttgtgatctactgctaattaaagttgggtgggcaggtggggatgccctgtattggggaatgtagtatactgtacatctaaaagcaattttggcaaaaccatctctgtcactgatctctcacttcacaatactaacatatagagttggggccaaagaagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagaccttcatcactccctagtgagggcgcggagctagatatgcaaaggatttatataaatttatgtgatacattttttttagttaattttaaaaagaaaaaaagatttatattttttaccaagatcaaattgaaatcacacaaatatttgtgaatttagatcataaatttcaaaaatcttttaataaaaggttagcttctgttcttggccttttagaggttaggtacatgctccatcccctcctttcttgttgttactatttgtcatggccggtagggatcaaggtcaaaccccaatgcatcacaagtcagtatagtccgaccgtgacaattttctatgggggattgtgtagaatcaaatatctattcataaattgagacattgagacgaatattcttctttacattattgagacgaaaaaaatattatttttttaaaaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccgtgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacatttttttatttgagtcactaaatttagatatttctttgatctgccacataacataataaatgatttcaaactcttgtcggaacatatgactcttaataaaaggtggagaagggttgaaaactttactaaatttaacaagatcttaggtgtgtatttgactcatgttacaagattgaagtgtatttaagtttaattaatcaagtaaaaaggtatttttaaggttgtcaatatataattcgaaaataaaactaataattcacattgaatttagagggtgttttcgatacttttcttttgtttcatttgttgagtttaaaattaagttgatatgaaacaacaaataaattgtgatcccaagttttatatgagcaaaataaaatcaagcattgtatatctctcttttatttttctccctggtgtccgcattggagcacgactatattcgaatcacactataggcttcatttttttcatactcagggcttaaatctgaaacttgtgggtaagggtagaatatcttatatcttatatctctttcttttttttttggattttccatccaacgtttgttacccacattgaagttcagttaattcaaatttgcgccgcataggctaagtggttcctaccaatgatctttctatacccagggattcaaatacaaaacatttatttaagaaataaatagtctcatccgttataccatatcagttgatagtgtggtatatctcattaagataccacattatatctatttaagggggtaggggggatgggtctagctagggttgtggaaatgaggttagagagagggtggtttttcagtgtgtgtgtcaggtgaaatcacgtgaaggggcacgataacaagttgttcatatccattcacttccaagaataccgctacgtacagtcactactgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctcactcacacagctagaatatatatatattatatatatattcatagttagcagaaatacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctactaactttaattttcttcttattgcagctagcttgcttaattagtagcaaaatcaaaagaggttttagctgtgtttatactgtctttctcaagatctagacccaccacttagaccatctcaagctacagctactgcgcaa

SEQ ID NO: 129 - DNA, 3000 bp, unidentified
  source 1..3000: mol_type = other DNA; note = the promoter regions of IT1 nucleotide sequences
agcacaaattttttaggttgccgatccagagaatttaaatattaatgttgtgaattctaatactccaaatattaatgttgtgaattctaatactccacctgttttaaaaaatggtctagtttgacttgaaacggaatttaagaaaagaaaaaagactttctaatcttgtgatgccaaattaaagttatgtcaaatataccaaaatgtcctttaatcttgtgctcttaaacatgtcacgtgaaaaattgaagttaaagtgttgccaaaaaaagaaaggggtcattattttttaaacaaactaaaaaaaaaaaactaagtcattccttttgaaacggagaaagtatactatatttatatatttggttctcgcaaacctgcaaataagggcggatgcacaatagaagttacgagtttgattaaatctactaattttatttaaactttgtatatatgtgtagagaagtcataaagttcaaatactgaatttactaattttatcaaagttgtgatctactgctaattaaagttgggtgggcaggtggggatgccccgtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaaggtcaaaagttgcattcttctcaaagggcaattcagactttcatcactccctagtgagggcgcggagctagatatgcaaagggtttatataaatttatgtgatacattttttttagttaatctaaaatataataaaagcttttttttttaacaagatcgatttgaaatcacacaaatatttgtgaatttagatcataaatttcaaaaatcttttaataaaaggctagcttctgttcttggccttttagaggttaggtacatgctccatcccctcctttcttgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacattcctttacttgacttactaaatttaaatatatcttcgatctgccacataacataacaaatggtttcaaacggaacatatgactcttaataaaaggtggagaagggttgaaaactttactaaatttaacaagatcttaggtgtgtatttgactcatgttgcaagattgaagtgtatttaagttcagttaatcaagtaaagagatatttttaaggttgtcaatatataattcggagataaaactagccttttgtttcatttgttgagtttaaaattaagttgatatgaaacaacaaataaattgtgatcccaagttttatatgagcaaaataaaatcaagcattgtatatctctcttttatttttctccctagtatccgcattggagcccgactatattcgaatcgcactataggcttcatttttttcatactcgagtcttaaatctgaaacctctgggtaagggtagaatatcttatatctctttttttttttttttggattttccattcaacgtttggtacccacattgaagttcagttaattcaaatttgcaccgcataggctaagcggttcctaccaatgatcttttcatacccagggactcaaacacaagaagtttaattaagaaataaatagtctcatccgctataccacattgtgatatatctcattaagataccacattatatctatttatgggggtaggggggatgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctctcactcacacagctagaatatatatatatatatatatatatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctaccaactttaattttcttcttattgcagctagcttgcttaattagcagcaaaactcaaaagaggttttagctgtgtttatactgtctttctcaagatctagacccaccacttagaccatctcaagctacagctactgcgcaa

SEQ ID NO: 130 - DNA, 3000 bp, unidentified
  source 1..3000: mol_type = other DNA; note = the promoter regions of IT1 nucleotide sequences
gattcggagggaaaatcatgttttatatcacaaattttttaggttgccgatccagagaatttaaatattaatgttgtgaattctaatactccacctgttttaaaaaatgatctagtttgacttgaaacggagtttaagaaaagaaaaaagactttctaatcttgtgattccaaattaaagttatatcaaatgtaccaaaatgtcctttaatcttgtggtcttaaatatgtcacgtgaaaaattgaagttaaagtgttgccaaaaaaagaaaggggtcattcttttttaaacaaactaaaaaagaaaactaagtcattccttttgaaacggaggaagtatactatatttatatatttggttctcgcaaacctgcaaataagggcggatgcacaacagaagttacgagtttgattaaatcttctaattttagtttaaactttgtatatatgcgtagagaagtcataaagttcaaatactgaatttactaattttatcaaagttgtgatctactgctgattaaagttgggtgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggttctctttttagtgaaagtgacatgctctaatgtcaaaagttgcattcttctcaaagggcaattcagactttcatcactccctagtgagggcgcggagctagatatgcaaagggtttatataaatttatgtgatacattttttttagttaatctaaaaaataataaaagctttttttttttaccaagatcgatttgaaatcacacaaatatttgtgaatttagatcataaatttcaaaagtcttttaataaaaggctaccttctgttcttggccttttagaggttaggtacatgctccatcccctcctttcttgttgttactatttgccatggccggtagggatcagggtcaaaccccaatgtatcacaggtcagtatagtccgaccgtgacaattttctatgggggattgtgtagaatcaaatatctattcataaattgagacattgagacgaatattcttctttacattattgagacgaaaaaaatattaaattttcttaataaaaatcttagctccccactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttacgacctagcctttctttttaagaagaataaaaaacatacctcgatttggtataaatcaatagtttcgtcaatcttaaaaacattcttttacttgatttattaaatttagatatatcttcgatctgccacataacataacaaatggtttcaaacggaacatatgactcttaataaaaggtggagaagggttgaaaactttactaaatttaacaagatcttaggtgtgtatttgactcatgttgcaagattgaagtgtatttaagttcagttaatcaagtaaatagatatttttaaggttgtcaatatataattcggagataaaactagccttttgtttcatttgttgagtttaaaattaagttgatatgaaacaacaaataaattgtgatcccaagttttatatgagcaaaataaaatcaagcattgtatatctctcttttatttttctccctggtatccgcattggagcctgactatatttgaatcgcactataggcttcatttttttcatactcgaggcttaaatctgaaacctctgggtaaggatagaatatcttatatctctttttttttttttttggattttccattcaacgtttggtacgcaaattgaagttcagttaattcaaatttgcaccgcataggctaagcggtttctaccaatgatcttttcatacccagggactcaaacacaagacgtttaattaagaagtaaatagtctcatccgctataccacattgtgtggtgatatatctcattaagataccacattatatctatttaagggggtaggggggatgggtctagctagggttgtggaaatgaggttagagagagagtggtttttcagtgtgtgtgtcaggtgatatcacgtgaaggggcacgataacaagttgttcatatccattcacttccaagaataccgctacgtacagtcactagctgctttcttcctatccccatctttgtcaaatttcctttccttttccaattactttttctttaatagacatggacgcaattcttccttcctttccccttcccctatattcttcctttatctctctttcacaataatagcaccatgcctgtagcttgatgcttagacgggtgcacacgcactctctcactcacacagctagaatatatatatatatatatatatattcatagttagcagaagtacttatcatataccaaaaaccacacaaatacattgtatcaagtgctgtcatactcaagcaaaagaaagaaaagaacaagatatagtactactgttttcatcaccattttggtcaatcatgatgattctgaacaaagatatagtactagctaggtagaaaataaatctaccaactttaattttcttcttattgcagctagcttgcttaattagcagcaaaactcaaaagaggttttagctgtgtttatactgtctttctcaagatctagacccaccacttagaccatctcaagctacagctactgcgcaa

SEQ ID NO: 131 - DNA, 3000 bp, unidentified
  source 1..3000: mol_type = other DNA; note = the promoter regions of IT1 nucleotide sequences
aatattaacgttgtgaattctaatactccacctgttttaaaaaatggtctagtttgactaggaacggagtttaagaaaaaaaaagattttctaatcttgtggttccaaattaaagttatgtcaaatgtaccaaaatgttctttaatcttgtggtcctaaacatgtcacttgaaaagttgaagctatttagtgttgccaaaaaaagaaaggagtcattctttttgaaacaagcaaaaaaaaaaactaagtcattctttttgaaacggagaaaatatactatatttatatatttggctctcgcaaacctgcaaataagggcggacgcacaacagaagttgcgagtttgattaaatctactaattttagtttaaactttgtatatatgtgtagaggagtcataaagttcaaatactgaatttattaattttatcaaagttgtgatctactgcttattaaagttgggtgggcaggtggggatgcccggtattggggaatgtagtatactgtacatctaaaagcaattttggcaaaaccatctctgtcactgatctctcacttcacaatactaacatatagagttggggccaaagtagtcatgtagttgatgggaagagatagtacggaggaatggagaaaaaggtaggtgggtgagttgggggaagagatagggtctctttttagtgaaagtgacatgctctaaggtcaaaagttgcattcttctcaaagggcaattcagaccttcatcactccctggtgagggcgcgcggagctagatatgcaaggagtttatataaatttatgtgatatattttttttaattaaactaaaaaggaataaaagtttttttttttttaccaagatcgatttgaaatcacacaaatatttgtgaatttagatcataaatttcaaaagtcttttaataaaaagctaggttctattcttggccttttagaggttagatacatgctccatccactccttccttgttgttattatttgccatgactggtagggatcaaggtcaaaccccaatgcatcacaggtcagtatagtccgaccgtgacaattttctaggggggattgtgtagaatcaaatatctattcataaattgagacattgagacgaatattcttcttcgcattattgagacaaagaaaatattaaattttattaataaaaatcttagctcctcactatttagtctaggtttcatattcaatgaattgctggggtttatgttattactcaactgaaataaaaagagaattgtcgttatccaggttagacaaaagcacgtgtagtttcaaacgcagtagccattattctcctctcctttacaggtgagattcaagggcagaaaggtaccatgcaagctgttatttaaatttacttattgattatgatcgacgactttgacagaaaaatgatgatgctttaattttgcgacctagcctttctttttaagaagaataaaaaacatacctcgatttggtataaatcaatagtttcgtcaaacttaaaaacattcttttacttgagtcactaaatttagatatttctttgatctgtcacataacataacaaatgatttcaaactcttgtcggaacatatgactcttaataaaaggtggagaagggttgaaaactttactaaatttaacaagatcttaggtgtgtatttgactcatgttgcaagattgaagtgtatttaagtttagttaatcaagtaaagagctatttttaaggttgtcaatatataattcggagataaaactaataattcacactgaatttagggggtgttttcaatactcttcttttgtttcatttcttgagtttaaaattaagttgatatgaaacaacaaataaattgtgatcccaagttttatatgagcaaaataaaatcaagcattatatatctctcttttatttttctccctagtgtccgcattggagcccgactatattcgaatcgcactataggctttatttttttcatactcagggcttaaatctgaaacatgtgggtaagggtagaatatcttatatctctttttttttttttgtttggattttccatccaacgtttgttacccacattgaagttcagttaattcaaatttgcgctgcataggctaagtggttcctaccaatgatcttttcatacccaatgactcaaacgcaaaacatttatttaagaaataaatagtctcatccgttataccacatcaattgatagtgtgatggagtatatctcattaagataccacattatatctatttgagggggtaggggggatgggtctagctagggttgtggaaatgaggttagagagagggtggtttttcagtgtgtgtgtcaggtgaaatcacgtgaaggggcacgataacaagttgttcatatccattcacttccaagaataccgctacgtacagtcactagctgctttcttcctatccccatctttgtcaaatttcctttccttttccaattactttttctttgatagacatggacgcaatttttccttcctttccccttcccctatattcttcctttatctctctttcacaataatagcaccatgcctgtagcttgatgcttaagacgggtgcacacgcactctcactcacacaactagaatatatatatatatatatatatatatattcatagttagcagaagtacttatcatataccaaaaaccacacaaatacattgtatcaagtgctgtcatactcaagcaaaaggaagaaaagaacaagatatagtactactgttttcatcaccattttggtcaatcatgatgattctgaacaaagatatagtactagctaggtagaaaataaatctactaactttaattttcttcttattgcagctagcttgcttaattagtagcaaaatcaaaagaagaggttttagctgtgtttatactgtctttctcaagatctagacccaccacttagaccatctcaagctacagctactgcgcaa

SEQ ID NO: 132 - DNA, 45 bp, unidentified
  source 1..45: mol_type = other DNA; note = target site 1 of a wild-type line
agcagaattagtattattagtggacggatctcgaaaaacaataag

SEQ ID NO: 133 - DNA, 46 bp, unidentified
  source 1..46: mol_type = other DNA; note = target site 1 of a mutant line
agcagaattagtattattagttggacggatctcgaaaaacaataag